(19) United States
(12) Patent Application Publication
(10) Pub. No.: US 2023/0068021 A1
(43) Pub. Date: Mar. 2, 2023

(54) INVAGINATING DEVICE, A SUTURING APPARATUS, A SUTURING INSTRUMENT AND A METHOD FOR SUTURING A LUMEN, VESSEL OR ORGAN

(71) Applicant: PALLIARE LIMITED, Galway (IE)

(72) Inventors: John O'DEA, County Galway (IE); Hilary Elizabeth Barrett, County Limerick (IE)

(73) Assignee: PALLIARE LIMITED, Galway (IE)

(21) Appl. No.: 17/899,946

(22) Filed: Aug. 31, 2022

(30) Foreign Application Priority Data
Sep. 1, 2021 (IE) S2021/0150
Nov. 22, 2021 (IE) S2021/0196
Jul. 6, 2022 (IE) S2022/0112

(51) Int. Cl. A61B 17/04; A61B 1/273; A61B 1/313

(52) U.S. Cl. CPC A61B 17/0469; A61B 17/0401; A61B 1/2736; A61B 1/313; A61B 2017/06009

(57) ABSTRACT

A suturing apparatus comprises an invaginating device secured to a distal end of an endoscope for invaginating two parts of the interior of a wall of a vessel, for example, a stomach to be sutured together to form a gastric sleeve. The invaginating device includes a suction chamber for forming invaginated parts of the interior of the wall of the vessel. A suturing instrument extending through an instrument channel of the endoscope extends into the invaginating device, and is urged into or adjacent the suction chamber for inserting a suture into the invaginated part in the suction chamber. The next invaginated part is then formed, and the suture is inserted into it. The suture is then tightened to draw the two invaginated parts together.

### FIELD OF THE INVENTION

The present invention relates to an invaginating device for use in a procedure for suturing a lumen, vessel or organ in a cavity in the body of a human or animal subject, either interiorly in the lumen, vessel or organ or exteriorly of the lumen, vessel or organ in the cavity in the body of the human or animal subject. The invention also relates to use of the invaginating device in a procedure for endoscopically suturing two portions of a lumen, vessel or organ together interiorly in the lumen, vessel or organ, for example, for suturing two portions of the wall of the stomach of a human or animal subject together in a sleeve gastroplasty procedure, for example, in an endoscopic sleeve gastroplasty procedure. The invention also relates to a suturing apparatus for use in a procedure for suturing two portions of a lumen, vessel or organ in a cavity in the body of a human or animal subject, either interiorly in the lumen, vessel or organ or exteriorly of the lumen, vessel or organ in the cavity of the body of the human or animal subject. The invention additionally relates to a suturing apparatus for use in a procedure for suturing two portions of a lumen, vessel or organ together interiorly in the lumen, vessel or organ, for example, for suturing two portions of the wall of a stomach of a human or animal subject together in a sleeve gastroplasty procedure, for example, an endoscopic sleeve gastroplasty procedure. The invention also relates to a method for suturing two portions of a lumen, vessel or organ in a cavity in the body of a human or animal subject, either interiorly in the lumen, vessel or organ or exteriorly of the lumen, vessel or organ in the cavity of the human or animal body. The invention further relates to a method for suturing two portions of an interior of a lumen, vessel or organ interiorly in the lumen, vessel or organ, for example, two portions of the wall of a stomach of a human or animal subject together in a sleeve gastroplasty procedure, for example, an endoscopic sleeve gastroplasty procedure. The invention also relates to an invaginating device for invaginating tissue and/or a part of a wall of a lumen, vessel or organ in the body of a human or animal subject.

### BACKGROUND TO THE INVENTION

In general, when it is necessary to suture the interior of a lumen, vessel or organ, for example, to suture two portions of the interior wall of a lumen, vessel or organ interiorly in the lumen, vessel or organ, an endoscope or other access device is inserted through an orifice or an incision in the body of a subject into the lumen, vessel or organ. A suturing instrument is then inserted into the lumen, vessel or organ through an instrument channel of the endoscope or other access device. In general, suturing of the two portions of wall of the lumen, vessel or organ requires the endoscope or other access device to approach the locations of the wall to be sutured substantially perpendicularly. Additionally, in many such known methods, in general, it is necessary to puncture the wall of the lumen, vessel or organ with a suturing needle of the suturing instrument such that the suturing needle extends externally through the wall of the lumen, vessel or organ, with the risk of puncturing an adjacent lumen, vessel or organ with potentially fatal results.

This is undesirable, and there is therefore a need, a method, apparatus and devices to address these problems.

### SUMMARY OF THE INVENTION

According to the invention there is provided an invaginating device for invaginating tissue and/or a part of a wall of a lumen, vessel or organ in the body of a human or animal subject, the invaginating device comprising:
a body member,
the body member comprising a connector adapted for coupling the body member to an access device adjacent a distal end thereof, the body member having
a suction chamber therein for invaginating the tissue and/or the part of the wall of the lumen, vessel or organ, and
an access opening to the suction chamber for accommodating the tissue and/or the part of the wall of the lumen, vessel or organ into the suction chamber for invaginating thereof,
the body member being adapted to accommodate a suturing instrument into or adjacent the suction chamber from a channel of the access device or from a channel extending through an elongated tubular member extending along and adjacent the access device.

In one embodiment of the invention the suction chamber communicates with an instrument channel in the access device.

In another embodiment of the invention the suction chamber is adapted to communicate with a channel of the access device or the channel extending through the tubular member for applying suction to the suction chamber for invaginating the tissue or the part of the wall of the lumen, vessel or organ.

In one embodiment of the invention the body member extends from a proximal end to a distal end, and defines a main axis extending from the proximal end to the distal end, the connector being located adjacent the proximal end of the body member, and being adapted to connect the body member to the access device with the main axis of the body member extending substantially parallel to a longitudinally extending axis of the access device adjacent the distal end thereof.

In one embodiment of the invention the suction chamber extends into the body member transversely of the main axis defined by the body member, and preferably, the access opening to the suction chamber faces in a direction transversely of the main axis defined by the body member, and advantageously, in a transverse direction opposite to the transverse direction into which the suction chamber extends into the body member.

In an alternative embodiment of the invention the suction chamber extends into the body member from the distal end thereof in an axial direction, and preferably, in an axial direction substantially parallel to the main axis defined by the body member.

In one embodiment of the invention the access opening to the suction chamber faces in a direction substantially parallel to the main axis of the body member, and preferably, the access opening is defined in the distal end of the body member, and advantageously, in a distal end face of the body member, the distal end face extending transversely of the main axis defined by the body member.

In another embodiment of the invention the invaginating device further comprises an instrument passageway extending through the body member from the suction chamber to the connector for accommodating the suturing instrument therethrough to the suction chamber, and preferably, the instrument passageway extends parallel to the main axis defined by the body member, and advantageously, the instrument passageway terminates in the suction chamber.

In another embodiment of the invention the instrument passageway communicates with the instrument channel of the access device.

In another embodiment of the invention the suction chamber and the instrument passageway communicate with respective instrument channels in the access device.

In one embodiment of the invention the instrument passageway is configured to accommodate the suturing instrument directly into the suction chamber.

In another embodiment of the invention the instrument passageway is configured to accommodate the suturing instrument to the suction chamber.

In a further embodiment of the invention the instrument passageway is configured to guide the suturing instrument to or into the suction chamber.

In another embodiment of the invention the instrument passageway communicates with the instrument channel of the access device, and preferably, the instrument passageway communicates with the instrument channel of the access device through the connector.

In another embodiment of the invention the instrument passageway terminates in the connector.

In one embodiment of the invention the instrument passageway extends through the body member from the distal end thereof to the proximal end thereof for accommodating an instrument through the body member.

In another embodiment of the invention a communicating passageway extends from the suction chamber for communicating with a suction source, and in one embodiment of the invention the communicating passageway is adapted to communicate the suction chamber with the suction source through the instrument channel of the access device.

Preferably, the communicating passageway is configured to communicate the suction chamber with the instrument channel of the access device through the connector.

In one embodiment of the invention the communicating passageway terminates in the connector.

Preferably, the communicating passageway comprises a communicating bore extending through the body member.

In an alternative embodiment of the invention the communicating passageway is adapted to communicate with the suction source through a conduit or a tubular member secured to and extending longitudinally along and externally of the access device.

In one embodiment of the invention the conduit or the tubular member is releasably secured to the access device, and preferably, is secured to the access device by a plurality of clips spaced apart longitudinally along the access device.

In one embodiment of the invention the instrument passageway is adapted to accommodate a suturing instrument secured to and extending longitudinally along and externally of the access device.

Preferably, the suturing instrument is releasably secured to the access device, and preferably, is secured to the access device by a plurality of clips spaced apart longitudinally along the access device.

In another embodiment of the invention the body member is of circular external transverse cross-section.

The invention also provides a combination of an access device and the invaginating device coupled to the access device adjacent a distal end of the access device. Preferably, the invaginating device is releasably coupled to the access device.

In one embodiment of the invention the access device comprises a single channel access device, and in an alternative embodiment of the invention the access device comprises a dual channel access device.

In one embodiment of the invention the access device comprises an endoscope.

The invention also provides an invaginating device comprising a body member having a proximal end and a distal end, the proximal end being adapted for coupling to an access device, such as an endoscope adjacent a distal end thereof, a suction chamber extending into the body member for invaginating tissue or a wall of a lumen, vessel, organ or cavity of a subject into the suction chamber when suction is applied to the suction chamber, and an instrument passageway for guiding a suturing instrument towards the suction chamber to pierce into the invaginated part of the tissue or wall in the suction chamber for inserting a part of a suture therein.

Preferably, the instrument passageway guides the suturing instrument so that the suturing instrument pierces through the tissue or the wall of the invaginated part.

In one embodiment of the invention the body member defines a main axis extending in a proximal/distal direction

Preferably, the suction chamber is located towards the distal end of the body member, and advantageously, the suction chamber is located adjacent the distal end of the body member.

In another embodiment of the invention the suction chamber is located intermediate the proximal and distal ends of the body member.

In another embodiment of the invention the suction chamber extends into the body member in a direction transversely of a proximal/distal direction relative to the body member.

In an alternative embodiment of the invention the suction chamber extends into the body member in a direction parallel to the proximal/distal direction relative to the body member.

In another embodiment of the invention the suction chamber defines an access opening, and in one embodiment of the invention the access opening faces in a direction transversely of the proximal/distal direction relative to the body member.

In an alternative embodiment of the invention the access opening defined by the suction chamber faces in a direction parallel to the proximal/distal direction of the body member.

Advantageously, the suction chamber is defined by a cavity formed in and extending into the body member from a side thereof. Alternatively, the suction chamber is defined by a cavity formed in and extending into the body member from the distal end of the body member.

In one embodiment of the invention the suction chamber is configured to communicate with a channel of an access device through which suction is applied to the suction chamber. In another embodiment of the invention the suction chamber is configured to communicate with a channel of an endoscope. Preferably, the suction chamber is configured to communicate with the channel of the access device adjacent the distal end of the endoscope or the access device, and advantageously, the suction chamber is configured to communicate with the channel of the endoscope or the access device through the body member.

Preferably, the suction chamber is configured to communicate with an instrument channel in the access device.

In one embodiment of the invention a communicating duct extends from the suction chamber for communicating the suction chamber with the channel of the endoscope or the access device.

Preferably, the communicating duct extends through the body member from the suction chamber to the proximal end of the body member.

Preferably, the instrument passageway extends in a proximal/distal direction relative to the body member.

In another embodiment of the invention the instrument passageway terminates adjacent the suction chamber.

In another embodiment of the invention the instrument passageway extends through a guide flange extending transversely from the body member intermediate the suction chamber and the proximal end of the body member, and preferably, adjacent the suction chamber.

Preferably, the instrument passageway comprises a guide bore extending through the guide flange, and advantageously, the instrument passageway extending through the guide flange extends substantially parallel to the proximal/distal direction of the body member.

In another embodiment of the invention the guide flange extends radially outwardly from the body member to one side thereof.

In an alternative embodiment of the invention the instrument passageway extends through the body member from the proximal end thereof.

In one embodiment of the invention the instrument passageway terminates in the suction chamber.

In another embodiment of the invention the instrument passageway is adapted to communicate with an instrument channel of the endoscope or the access device.

In one embodiment of the invention the instrument passageway extends in an upstream/downstream direction, and preferably, parallel to the main axis defined by the body member.

In another embodiment of the invention the instrument passageway comprises a distal guide channel extending proximally from the suction chamber, and preferably, the guide channel defines an elongated open mouth extending proximally from the access opening of the suction chamber, and preferably, the open mouth of the guide channel and the access opening of the suction chamber face in a common direction.

In another embodiment of the invention the instrument passageway comprises a proximal guide bore communicating the guide channel with the proximal end of the body member.

In a further embodiment of the invention a distal instrument accommodating bore extends distally from the suction chamber through the body member and through the distal end of the body member, the distal instrument accommodating bore being aligned with the instrument passageway for accommodating an instrument therethrough for tightening a suture.

In one embodiment of the invention a connector is provided for connecting the body member to the endoscope or the access device, and preferably, the connector is located adjacent the proximal end of the body member, and advantageously, the connector comprises a socket for engaging the endoscope or the access device adjacent a distal end of the endoscope or the access device, and preferably, the socket extends into the body member from the proximal end thereof.

In one embodiment of the invention the instrument passageway extends to the connector, and preferably, the instrument passageway communicates with the instrument channel of the endoscope or the access device through the connector.

Preferably, the communicating duct communicates with the instrument channel of the endoscope or the access device through the connector through which suction is applied to the suction chamber.

In another embodiment of the invention the body member is adapted to permit viewing of the interior of the lumen, vessel, organ or cavity through a vision channel of the endoscope or the access device.

In another embodiment of the invention the body member comprises a transparent material to permit viewing of the interior of the lumen, vessel, organ or cavity therethrough through the vision channel of the endoscope or the access device.

In another embodiment of the invention the body member comprises a viewing aperture formed therein adapted to communicate with the vision channel of the endoscope or the access device to permit viewing of the interior of the lumen, vessel, organ or cavity through the vision channel.

In a further embodiment of the invention a viewing recess is formed in the body member adapted to communicate with the vision channel of the endoscope or the access device, and preferably, the viewing recess terminates in the viewing aperture.

Preferably, the viewing recess extends distally along the body member from the viewing aperture, and advantageously, the viewing recess extends to the distal end of the body member.

In one embodiment of the invention the viewing aperture comprises an elongated viewing bore extending from the proximal end of the body member, and preferably, the viewing bore extends to the distal end of the body member, and advantageously, the viewing bore extends in the proximal/distal direction relative to the proximal and distal end of the body member.

In another embodiment of the invention the viewing bore extends substantially parallel to the main axis defined by the body member, and advantageously, the viewing bore extends from the proximal end of the body member to the distal end thereof.

In another embodiment of the invention the viewing aperture communicates with the vision channel of the endoscope or the access device through the connector.

In one embodiment of the invention a sealing means is provided for sealably coupling the communicating duct to the instrument channel of the endoscope or the access device.

In one embodiment of the invention the body member is of circular transverse cross-section, and preferably, the body member defines the main axis as a central main axis extending in a proximal/distal direction.

In one embodiment of the invention the body member comprises a polymer material.

In another embodiment of the invention the body member comprises a translucent material.

Preferably, the body member comprises a lubricious material that slides easily over the distal end of an endoscope or the access device.

Alternatively, the body member comprises a plastics material.

In one embodiment of the invention the body member comprises an elongated body member, and preferably, the body member is of substantially constant transverse cross-section.

In another embodiment of the invention the body member is of circular, or partly circular, transverse cross-section.

In another embodiment of the invention the body member defines a cylinder or a part thereof.

In another embodiment of the invention the invaginating device is adapted for invaginating tissue of a lumen, vessel or organ.

In one embodiment of the invention the invaginating device is adapted for invaginating the tissue of the lumen, vessel or organ interiorly in the lumen, vessel or organ.

In an alternative embodiment of the invention the invaginating device is adapted for invaginating the tissue of the lumen, vessel or organ exteriorly of the lumen, vessel or organ in a cavity in the body of a human or animal subject.

In another embodiment of the invention the invaginating device is adapted for invaginating an inner part of the wall of a stomach, and preferably, the invaginating device is adapted for invaginating the inner part of the wall of the stomach in a sleeve gastroplasty procedure.

In another embodiment of the invention the invaginating device is adapted for invaginating an inner part of the wall of a stomach in an endoscopic sleeve gastroplasty procedure.

Additionally, the invention provides a suturing instrument comprising:
an elongated cannula extending from a proximal end to a distal end and having a suture accommodating bore extending longitudinally therethrough for accommodating a plurality of sutures therein,
the cannula terminating at its distal end in a distal piercing tip,
the sutures comprising:
   a suturing thread terminating at its opposite ends in respective anchor elements,
   the suture thread being configured to form a loop extending from one anchor element to the other one of the anchor elements, and
   a cinch clip located on the loop of the suture thread and being urgeable along the loop towards the anchor elements for progressively reducing the effective length of the suture thread between the anchor elements,
   the suture accommodating bore being configured to accommodate the sutures therein with at least the anchor elements of the sutures being located in the suture accommodating bore, and
   a pusher element for selectively and sequentially discharging the anchor elements of the suture from the suture accommodating bore through the distal piercing tip of the cannula.

In one embodiment of the invention the pusher element is slideable in the suture accommodating bore.

In one embodiment of the invention the pusher element extends into the suture accommodating bore from the proximal end of the cannula.

In another embodiment of the invention an urging means is provided for urging the pusher element distally in the suture accommodating bore for selectively and sequentially discharging the anchor elements from the suture accommodating bore.

In one embodiment of the invention the suture accommodating bore is configured for accommodating the anchor elements of each suture aligned with each other, and preferably, for accommodating the anchor elements of the respective sutures aligned with each other, and advantageously, for accommodating the anchor elements aligned with the suture accommodating bore.

In another embodiment of the invention the suture accommodating bore is configured for accommodating elongated anchor elements of the respective sutures aligned longitudinally with each other.

In another embodiment of the invention the suture accommodating bore is configured for accommodating the anchor elements of the respective sutures aligned end-to-end with each other.

In another embodiment of the invention the suture accommodating bore is configured for accommodating the cinch clips of the respective sutures, and preferably, the suture accommodating bore is configured for accommodating elongated cinch clips, and preferably, the suture accommodating bore is configured to accommodate the cinch clips of the respective sutures longitudinally aligned with the suture accommodating bore, and preferably, longitudinally aligned with each other in the suture accommodating bore.

In another embodiment of the invention the suture accommodating bore is configured to accommodate the cinch clips aligned with the anchor elements of the respective sutures. Preferably, the suture accommodating bore is configured for accommodating the cinch clips of the respective sutures aligned end-to-end with adjacent anchor elements.

In another embodiment of the invention the suture accommodating bore is configured to accommodate the cinch clip of each suture located between the anchor elements of the corresponding suture.

In an alternative embodiment of the invention the suture accommodating bore is configured for accommodating the cinch clip of each suture located distally relative to the anchor elements of the corresponding suture.

In a further alternative embodiment of the invention the suture accommodating bore is configured for accommodating the cinch clip of each suture located proximally relative to the anchor elements of the corresponding suture.

In another embodiment of the invention the pusher element is configured to selectively and sequentially urge the cinch clips and the anchor elements of the respective suture, from the suture accommodating bore through the distal piercing tip of the cannula.

In another embodiment of the invention an elongated longitudinal slot extends longitudinally along the cannula and through the cannula from an outer side to an inner side of the cannula wall for accommodating the suture threads of the respective sutures therethrough.

In one embodiment of the invention the cannula terminates in a distal piercing portion extending proximally from the distal piercing tip, and preferably, the distal piercing portion is substantially rigid for piercing through tissue.

In another embodiment of the invention the cannula comprises an intermediate portion extending from the distal piercing portion to a proximal portion adjacent the proximal end of the cannula. Preferably, the intermediate portion of the cannula is configured to be flexible and bendable for flexing and bending to accommodate maneuvering of the suturing instrument through various bends and curves through which the suturing instrument must be passed to gain access to a lumen, vessel, organ or cavity in which suturing is to be carried out.

In one embodiment of the invention an interrupted helical slot extends through the wall of the intermediate portion of the cannula, and the interrupted helical slot extends along the intermediate portion thereof. Preferably, the interrupted helical slot extends the length of the intermediate portion of the cannula from the distal piercing portion to the proximal portion thereof.

In one embodiment of the invention the interrupted helical slot comprises a plurality of slot sections extending partly around the wall of the intermediate portion of the cannula with interrupted sections of the interrupted helical slot located between adjacent pairs of the slot sections.

In one embodiment of the invention the interrupted helical slot is of pitch lying in the range of 0.8 mm to 1.5 mm, and preferably, the pitch of the interrupted helical slot lies in the range of 1.1 mm to 1.3 mm, and advantageously, the pitch of the interrupted helical slot is approximately 1.2 mm.

In one embodiment of the invention each slot section extends partly around the intermediate portion of the cannula an angular distance in the range of 170° to 350°. Preferably, each slot section extends partly around the intermediate portion of the cannula an angular distance in the range of 210° to 290°. Advantageously, each slot section extends partly around the intermediate portion of the cannula an angular distance in the range of 220° to 260°. Preferably, each slot section extends partly around the intermediate portion of the cannula an angular distance of approximately 240°.

In another embodiment of the invention each interrupted section of the interrupted helical slot extends partly around the intermediate portion of the cannula an angular distance in the range of 10° to 190°. Preferably, each interrupted section of the interrupted helical slot extends partly around the intermediate portion of the cannula an angular distance in the range of 20° to 130°. Advantageously, each interrupted section of the interrupted helical slot extends partly around the intermediate portion of the cannula an angular distance in the range of 30° to 70°, and ideally, each interrupted section of the interrupted helical slot extends partly around the intermediate portion of the cannula an angular distance of approximately 40°.

In another embodiment of the invention the kerf width of the slot sections of the interrupted helical slot is approximately 0.025 mm.

In one embodiment of the invention the outer diameter of the cannula lies in the range of 0.7 mm to 1.8 mm, and preferably, is approximately 1.27 mm.

In another embodiment of the invention the wall thickness of the cannula lies in the range of 0.05 mm to 0.3 mm, and preferably, the wall thickness of the cannula is approximately 0.0762 mm.

In another embodiment of the invention the suture accommodating bore of the cannula is of transverse cross-section just slightly greater than the maximum transverse cross-sectional dimension of the anchor elements or the cinch clip, whichever of which the maximum transverse cross-sectional dimension is the greatest.

In one embodiment of the invention a retaining means is provided for releasably retaining the sutures in the suture accommodating bore, and preferably, the retaining means is located adjacent the distal end of the cannula, and advantageously, the retaining means is located just slightly spaced apart longitudinally and proximally from the distal piercing tip.

In one embodiment of the invention the retaining means comprises a retaining projection extending inwardly into the suture accommodating bore, preferably the retaining projection is formed by inwardly impressing a dimple into the cannula wall from the outer surface thereof, and preferably, a portion of the cannula adjacent which the dimple is impressed into the wall of the cannula is configured to be resilient to permit outward movement of the portion adjacent which the dimple is impressed, and in turn outward movement of the retaining projection, in order to accommodate passage of the distal most one of the anchor elements or the cinch clip of the distal most suture in the suture accommodating bore past the retaining projection in response to distal urging thereof by the pusher element. Preferably, the resilience of the portion of the cannula adjacent the impressed dimple is such as to return the portion to its undisturbed state for in turn returning the retaining projection to engage and retain captive the next distal most one of the anchor elements or the cinch clip of the distal most suture or of the next distal most suture in the suture accommodating bore.

In another embodiment of the invention the urging means is configured for selectively and sequentially urging the anchor elements and the cinch clips of the respective sutures from the suture accommodating bore through the distal piercing tip of the cannula.

Preferably, the urging means is configured to urge the pusher element distally selectively and sequentially through a plurality of distances for selectively and sequentially discharging the anchor elements and/or the anchor elements and the cinch clips of the respective sutures through the distal piercing tip of the cannula.

In one embodiment of the invention the distance through which the urging means is configured to urge the pusher element distally in the suture accommodating bore for discharging each one of the anchor elements and/or each one of the cinch clips from the suture accommodating bore corresponds to the length of the distal most one of the anchor elements and/or the cinch clip of the distal most suture in the suture accommodating bore, and advantageously, the distance through which the urging means is configured to urge the pusher element distally in the suture accommodating bore for discharging each one of the anchor elements and/or each one of the cinch clips therefrom is substantially equal to the length of the distal most one of the anchor elements and/or the cinch clip of the distal most suture in the suture accommodating bore of the cannula.

In another embodiment of the invention the urging means is configured to urge the pusher element distally in the suture accommodating bore for discharging each one of the anchor elements and/or the cinch clips from the suture accommodating bore respective first, second and third predefined distances substantially equal to the respective lengths of the anchor elements and the length of the cinch clip.

In one embodiment of the invention the first and second predefined distance are of similar length, and preferably, are of equal length, and advantageously, the third predefined distance is of a substantially similar length to the first predefined distance, and advantageously, the third predefined distance is of length substantially equal to the first predefined distance.

In one embodiment of the invention the urging means comprises a linear motor, and in another embodiment of the invention the urging means comprises a stepper motor.

In a further embodiment of the invention a controller is provided for controlling the operation of the urging means, and preferably, the controller is responsive to an activation signal for operating the urging means to urge the pusher element distally in the suture accommodating bore through each one of the distances corresponding to the length of the distal most one of the anchor elements and/or the cinch clip of the distal most suture in the suture accommodating bore.

Preferably, the controller is responsive to the activation signal for operating the urging means to urge the pusher element distally in the suture accommodating bore through the first predefined distance for urging a first one of the anchor elements of the sutures from the suture accommodating bore of the cannula through the distal piercing tip thereof.

In another embodiment of the invention the controller is responsive to the activation signal for operating the urging means to urge the pusher element distally in the suture accommodating bore through the second predefined distance for discharging a second one of the anchor elements of the sutures from the suture accommodating bore of the cannula through the distal piercing tip thereof.

In a further embodiment of the invention the controller is responsive to the activation signal for urging the pusher element distally in the suture accommodating bore through the third predefined distance for urging the cinch clip of the sutures from the suture accommodating bore of the cannula therefrom through the distal piercing tip.

In one embodiment of the invention the activation signal comprises an electrical signal, and preferably, is derived from a human operable switch.

In one embodiment of the invention the human operable switch comprises a foot operable switch, and preferably, a switch operable by a foot pedal.

In another embodiment of the invention the human operable switch comprises a manually operable switch.

In one embodiment of the invention the controller is programmed to operate the urging means to urge the distal most pusher element distally in the suture accommodating bore one of the first predefined distance of the predefined distances to discharge one of the anchor elements adjacent the distal piercing tip from the suture accommodating bore through the distal piercing tip.

In another embodiment of the invention the controller is programmed to operate the urging means to urge the pusher element distally in the suture accommodating bore one of the second predefined distance of the predefined distances to discharge one of the cinch clips adjacent the distal piercing tip from the suture accommodating bore through the distal piercing tip.

In one embodiment of the invention the anchor elements of the respective sutures are of the same length.

In another embodiment of the invention the cinch clips of the respective sutures are of the same length.

In a further embodiment of the invention the cinch clips of the respective sutures are of the same length as the anchor elements thereof.

In one embodiment of the invention the second predefined distance is equal to the first predefined distance.

In another embodiment of the invention the controller is responsive to each activation signal for operating the urging means to urge the pusher element distally through one of the predefined distances.

In one embodiment of the invention the controller comprises a signal processor, and preferably, a programmable signal processor, and advantageously, the signal processor comprises a microcontroller.

In one embodiment of the invention the signal processor is programmed to count the anchor elements and/or the cinch clips, as they are being discharged from the suture accommodating bore, in order to determine the identity of the distal most one of the anchor elements and/or the cinch clip in the suture accommodating bore of the cannula.

In another embodiment of the invention a sensor is provided for detecting the distal most one of the anchor elements and the cinch clip of the distal most suture in the suture accommodating bore, the sensor being configured to produce a signal indicative of the one of the anchor element and the cinch clip which is distal most in the suture accommodating bore.

In one embodiment of the invention the sensor is located adjacent the distal end of the cannula, and preferably, the sensor is located in or on the cannula slightly spaced apart longitudinally and proximally from the distal piercing tip thereof.

In another embodiment of the invention the suturing instrument comprises an outer sleeve extending from a proximal end to a distal end and having a cannula bore configured for accommodating the cannula therein.

In one embodiment of the invention the cannula is slideable within the cannula bore.

In another embodiment of the invention the cannula is of a length greater than the length of the outer sleeve.

In an alternative embodiment of the invention the outer sleeve is of a length greater than the length of the cannula.

The invention also provides the suturing instrument according to the invention comprising a plurality of the sutures located in the suture accommodating bore.

In one embodiment of the invention each suture comprises a suturing thread terminating at its opposite ends in respective anchor elements, the suture thread being configured to form a loop between the anchor elements, and a cinch clip located on the loop of the suture thread and being urgeable along the loop towards the anchor elements for progressively reducing the effective length of the suture thread between the anchor elements.

In one embodiment of the invention the sutures are located in the suture accommodating bore with the anchor elements of each suture located in the suture accommodating bore and aligned with each other, and preferably, with the anchor elements of the respective sutures aligned with each other.

In another embodiment of the invention the anchor elements of the respective sutures comprise elongated anchor elements, and preferably, the sutures are located in the suture accommodating bore with the elongated anchor elements aligned longitudinally with each other.

In another embodiment of the invention the sutures are located in the suture accommodating bore with the anchor elements of the respective sutures aligned end-to-end with each other.

In another embodiment of the invention the sutures are located in the suture accommodating bore with the cinch clips of the respective sutures located in the suture accommodating bore.

In one embodiment of the invention the cinch clip of each suture comprises an elongated cinch clip, and preferably, the sutures are located in the suture accommodating bore with the cinch clips of the respective sutures longitudinally aligned with the suture accommodating bore, and preferably, longitudinally aligned with each other in the suture accommodating bore.

In another embodiment of the invention the sutures are located in the suture accommodating bore with the cinch clips aligned with the anchor elements of the respective sutures. Preferably, the sutures are located in the suture accommodating bore with the cinch clips of the respective sutures aligned end-to-end with adjacent anchor elements.

In another embodiment of the invention the sutures are located in the suture accommodating bore with the cinch clip of each suture located between the anchor elements of the corresponding suture.

In an alternative embodiment of the invention the sutures are located in the suture accommodating bore with the cinch clip of each suture located distally relative to the anchor elements of the corresponding suture.

In a further alternative embodiment of the invention the sutures are located in the suture accommodating bore with the cinch clip of each suture located proximally relative to the anchor elements of the corresponding suture.

In one embodiment of the invention the anchor elements of the respective sutures are of the same length.

In another embodiment of the invention the cinch clips of the respective sutures are of the same length.

In a further embodiment of the invention the cinch clips of the respective sutures are of the same length as the anchor elements thereof.

The invention also provides a suturing apparatus for suturing two portions of a lumen, vessel or organ in a cavity of the body of a human or animal subject either interiorly in the lumen, vessel or organ or exteriorly of the lumen, vessel or organ in the cavity in the body of the human or animal subject, the suturing apparatus comprising the invaginating device according to the invention for sequentially invaginating parts of the two portions of the lumen, vessel or organ, and a suturing instrument for inserting a suture into two invaginated parts of the respective portions of the lumen, vessel or organ. Preferably, the suturing instrument cooperates with the invaginating device for inserting the suture into the invaginated parts of the lumen, vessel or organ.

In one embodiment of the invention the suture comprises a suture thread terminating in its opposite ends in respective anchor elements, and forming a loop between the anchor elements to form a loop, and having a cinch clip located on the loop urgeable along the loop towards the anchor elements for progressively reducing the effective length of the suture thread between the anchor elements.

In another embodiment of the invention the suturing instrument comprises a cannula having a suture accommodating bore extending longitudinally therethrough for accommodating the anchor elements of the suture sequentially aligned therein and terminating at a distal end in a distal piercing tip, a pusher element urgeable distally in the suture accommodating bore for sequentially discharging the anchor elements from the suture accommodating bore through the distal piercing tip of the cannula, the distal piercing tip being adapted to extend into or adjacent the suction chamber of the invaginating device, so that when the distal piercing tip is urged through one of the invaginated parts in the suction chamber, and the pusher element is urged to discharge one of the anchor elements through the distal piercing tip of the cannula, the discharged anchor element is located on one side of the invaginated part with a part of the suture thread adjacent the anchor element extending from the anchor element through the invaginated part.

Preferably, the pusher element terminates in a distal abutment end, the pusher element being urgeable distally in the suture accommodating bore from a withdrawn state within the suture accommodating bore with the distal abutment end thereof defining with a distal portion of the suture accommodating bore an anchor element accommodating chamber within which the anchor elements are sequentially aligned for sequential discharge of the anchor elements from the anchor element accommodating chamber through the distal piercing tip of the cannula.

In one embodiment of the invention the suturing instrument comprises an outer sleeve defining a longitudinally extending cannula accommodating bore within which the cannula is located and is longitudinally slideable. Preferably, the cannula is urgeable distally in the cannula bore of the outer sleeve from a withdrawn state within the cannula bore with the distal end of the cannula defining with a distal portion of the outer sleeve, a cinch clip accommodating chamber for accommodating the cinch clip of the suture, for discharging the cinch clip from the cinch clip accommodating chamber through the distal end of the outer sleeve and for sequentially piercing the invaginated parts of the lumen, vessel or organ in the suction chamber.

Preferably, the cinch clip accommodating chamber is adapted for accommodating a part of the loop of the suture along with the cinch clip thereof.

In another embodiment of the invention the outer sleeve terminates in a distal abutment face, and preferably in an annular abutment face.

In one embodiment of the invention the pusher element is adapted for engaging the loop of the suture and for urging the cinch clip along the loop towards the anchor elements for progressively reducing the effective length of the suture thread between the anchor elements.

In one embodiment of the invention the pusher element terminates in a distal hook or grabber for engaging the loop of the suture and for urging the suture loop through the cinch clip for in turn urging the cinch clip along the loop towards the anchor elements to progressively reduce the effective length of the suture thread between the anchor elements.

Preferably, the distal hook or grabber of the pusher element defines the distal abutment end of the pusher element.

In one embodiment of the invention the invaginating device is adapted to guide the cannula of the suturing instrument to an invaginated part retained in the suction chamber for piercing through the invaginated part, and preferably, the pusher element is adapted for urging the distal most one of the anchor elements from the anchor element accommodating chamber for depositing the distal most one of the anchor element on a distal side of the invaginated part.

In another embodiment of the invention the invaginating device is adapted to accommodate the outer sleeve of the suturing instrument therein with the cannula urgeable from the outer sleeve for piercing through the invaginated part by the distal piercing tip of the cannula.

In one embodiment of the invention the outer sleeve of the suturing instrument is urgeable into the instrument passageway of the invaginating device.

In an alternative embodiment of the invention the suturing instrument of the suturing apparatus comprises the suturing instrument according to the invention, and the instrument passageway of the invaginating device is adapted for guiding the cannula of the suturing instrument according to the invention to the invaginated part retained in the suction chamber for inserting the suture into the invaginated part.

Preferably, the instrument passageway of the invaginating device is adapted to accommodate the outer sleeve of the suturing instrument according to the invention therein.

In one embodiment of the invention the cannula of the suturing instrument according to the invention is urgeable through the instrument passageway of the invaginating device for urging the distal piercing tip of the cannula through the invaginated part in the suction chamber.

In one embodiment of the invention the invaginating device is coupled to a distal end of an access device, and the suturing instrument is passed into or adjacent the suction chamber of the invaginating device through an instrument channel of the access device or through a tubular member extending along the access device.

In another embodiment of the invention the tubular member extending along the access device comprises a tubular member of the suturing instrument.

In one embodiment of the invention the access device comprises an endoscope.

In another embodiment of the invention the access device comprises a laparoscope.

The invention further provides an access device comprising the invaginating device coupled thereto, and preferably, coupled to the distal end of the access device.

In one embodiment of the invention the suction chamber is connected to a channel of the access device, and preferably, to an instrument channel adjacent the distal end of the access device.

In an alternative embodiment of the invention the suturing instrument is attached to the access device, and extends longitudinally along an outer side of the access device, and preferably, the suturing instrument is releasably attached to the access device, and preferably, the suturing instrument is releasably attached to the access device by a plurality of spaced apart clips spaced apart longitudinally along the access device.

In one embodiment of the invention the clips are releasably engageable with the access device, and in another embodiment of the invention the clips are releasably engageable with the suturing instrument.

In a further embodiment of the invention the clips are releasably engageable with both the suturing instrument and the access device.

In one embodiment of the invention the clips are retained together by a retaining means, and preferably, the retaining means comprises a ligature.

Additionally, the invention provides a method for suturing two portions of a lumen, vessel or organ in a cavity in the body of a human or animal subject either interiorly in the lumen, vessel or organ or exteriorly of the lumen, vessel or organ in the cavity, the method comprising:
entering an access device with the invaginating device according to the invention coupled to the distal end of the access device into the interior of the lumen, vessel or organ or into the cavity through a body orifice or through an incision in the body of the human or animal subject,
bringing the invaginating device into engagement with a surface of the wall of the lumen, vessel or organ with the suction chamber adjacent a first one of the portions to be sutured,
applying suction to the suction chamber to suction a part of the first one of the portions to be sutured into the suction chamber for invaginating thereof to form a first invaginated part,
urging a distal piercing tip of a cannula of a suturing instrument distally through the first invaginated part retained in the suction chamber,
operating the suturing instrument to discharge a first one of a pair of anchor elements of a suture from the suturing instrument to the distal side of the first invaginated part,
withdrawing the cannula proximally from the first invaginated part with a suture thread of the suture extending proximally from the first anchor element through the first invaginated part, and
releasing suction from the suction chamber to release the first invaginated part therefrom.

In one embodiment of the invention the method further comprises:
bringing the invaginating device into engagement with the surface of the wall of the lumen, vessel or organ with the suction chamber adjacent a second one of the portions of the lumen, vessel or organ to be sutured,
reapplying suction to the suction chamber to suction a part of the second portion to be sutured into the suction chamber for invaginating thereof to form a second invaginated part,
urging the distal piercing tip of the cannula of the suturing instrument through the second invaginated part retained in the suction chamber,
operating the suturing instrument to discharge a second one of the pair of the anchor elements of the suture from the suturing instrument to the distal side of the second invaginated part,
withdrawing the cannula proximally from the second invaginated part with the suture thread of the suture extending proximally from the second anchor element through the second invaginated part, and
releasing suction from the suction chamber to release the invaginated part therefrom.

In another embodiment of the invention the method comprises tightening the suture to draw the two invaginated parts together.

In another embodiment of the invention the suturing instrument is operated to discharge a cinch clip of the suture into the lumen, vessel, organ or cavity in which the suturing is being carried out, and preferably, the cinch clip is located on a loop of the suture thread of the suture extending from the respective anchor elements thereof.

In another embodiment of the invention the suture is tightened by urging the cinch clip of the suture along the loop thereof towards the anchor elements to reduce the effective length of the suture thread between the anchor elements to draw the two invaginated parts together.

In one embodiment of the invention the cinch clip is urged along the loop of the suture towards the anchor elements by drawing the loop into the outer sleeve of the suturing instrument or an outer sleeve of a tightening instrument with the cinch clip abutting the distal abutment face of the outer sleeve.

In another embodiment of the invention the loop of the suture is drawn into the outer sleeve of the suturing instrument by the pusher element, and preferably, the loop of the suture is engaged by a hook or a grabber element of the pusher element prior to being drawn into the outer sleeve of the suturing instrument.

In another embodiment of the invention the loop of the suture is drawn into the outer sleeve of the suturing instrument with the cinch clip abutting the distal abutment face of the outer sleeve for urging the cinch clip along the loop towards the anchor elements.

In one embodiment of the invention the loop of the suture is released from the suturing instrument or the tightening instrument.

In another embodiment of the invention the suturing instrument is withdrawn from the invaginating device for recharging with another suture after the current suture has been engaged in the two corresponding invaginated parts or on completion of urging of the cinch clip along the loop of the suture.

In one embodiment of the invention a plurality of sutures are inserted into respective pairs of invaginated parts of the portions of the lumen, vessel or organ to be sutured the pair of the invaginated parts being spaced apart along a line along which the portions of the lumen, vessel or organ are sutured together.

In one embodiment of the invention a plurality of the sutures are engaged in respective pairs of the invaginated parts of the lumen, vessel or organ prior to tightening of the sutures.

In one embodiment of the invention the sutures are sequentially tightened, and preferably, the sutures are sequentially from the distal most one of the sutures to the proximal most one of the sutures of the lumen, vessel or organ.

Alternatively, the sutures are tightened simultaneously.

In one embodiment of the invention the invaginating device is entered into the interior of the lumen, vessel or organ, and the invaginated parts formed in the lumen, vessel or organ extend into the interior of the lumen, vessel or organ.

In another embodiment of the invention the suturing instrument is entered into the interior of the lumen, vessel or organ, and suturing of the lumen, vessel or organ is carried out internally in the interior of the lumen, vessel or organ.

In an alternative embodiment of the invention the invaginating device is entered into the cavity, and the invaginated parts of the lumen, vessel or organ extend from the lumen, vessel or organ into the cavity.

In another embodiment of the invention the suturing instrument is entered into the cavity, and suturing of the lumen, vessel or organ is carried out in the cavity externally of the lumen, vessel or organ.

In one embodiment of the invention the invaginating device is coupled to an endoscope, and preferably, to the distal end of the endoscope.

In another embodiment of the invention the invaginating device is sealably coupled to the endoscope with the suction chamber communicating with a channel of the endoscope, and preferably, an instrument channel of the endoscope adjacent the distal end of the endoscope.

In another embodiment of the invention the suturing instrument is entered into the suction chamber of the invaginating device through an instrument channel of the access device or through a tubular member extending along an outer side of the access device.

In another embodiment of the invention the suturing instrument is secured to an outer side of the access device and extends longitudinally along the outer side of the access device, and preferably, the suturing instrument is secured to an outer side of the access device and extends longitudinally along the outer side of the access device, and preferably, a distal end of the suturing instrument terminates adjacent the invaginating device, and preferably, the distal end of the suturing instrument is urgeable into the suction chamber of the invaginating device, and preferably, the suturing instrument extends into the instrument passageway of the guide flange of the invaginating device.

In one embodiment of the invention the suturing instrument is releasably secured to the outer side of the access device, and preferably, is secured to the outer side of the access device by a plurality of clips spaced apart longitudinally along the access device.

In one embodiment of the invention the clips releasably engage the suturing instrument, and in another embodiment of the invention the clips releasably engage the access device.

Preferably, the clips releasably engage the suturing instrument and the access device.

In another embodiment of the invention the clips are connected together, and preferably, are connected together by a retaining means, and preferably, the retaining means comprises a ligature.

In one embodiment of the invention the invaginating device is secured to the distal end of an endoscope, and the suturing instrument is secured to the outer side of the endoscope, and extends longitudinally along the outer side of the endoscope.

In one embodiment of the invention the lumen, vessel or organ to be sutured comprises the stomach of the subject, and preferably, the invaginating device is entered orally through the oesophagus into the interior of the stomach.

In one embodiment of the invention the method is adapted for carrying out a sleeve gastroplasty procedure, and preferably, a plurality of pairs of invaginated parts are formed on the front and rear walls of the stomach, with one of each pair of invaginated parts being formed on the front wall of the stomach, and the other one of each pair of the invaginated parts being formed on the rear wall of the stomach. Preferably, the pairs of the invaginated parts are formed at spaced apart intervals along a line along which the front and rear walls of the stomach are to be sutured to form a gastric sleeve of the sleeve gastroplasty procedure.

In another embodiment of the invention the procedure carried out in the stomach is an endoscopic sleeve gastroplasty procedure.

In one embodiment of the invention a plurality of pairs of spaced apart invaginated parts of the lumen, vessel or organ spaced apart along a line of the pairs of the parts are sutured.

In another embodiment of the invention the invaginated parts of each pair thereof are located on respective opposite sides of an incision in the wall of the lumen, vessel or organ, and in another embodiment of the invention the invaginated parts of each pair thereof are located on respective opposite sides of a weakened region of the wall of the lumen, vessel or organ resulting from scraping or otherwise removing a part of the tissue of the wall of the lumen, vessel or organ extending into the wall of the lumen, vessel or organ from an inner surface or an outer surface of the wall thereof, or the invaginated parts of each pair thereof to be sutured together are located on respective opposite sides of an incision formed in the wall of the lumen, vessel or organ.

The invention further provides a method for suturing two portions of a lumen, vessel or organ in a cavity in the body of a human or animal subject, either internally in the interior of the lumen, vessel or organ, or externally of the lumen, vessel or organ in the interior of the cavity, the method comprising:
invaginating at least one part of each of the portions to be sutured of the lumen, vessel or organ to form a pair of the invaginated parts, and
suturing the invaginated parts of each pair thereof together.

In one embodiment of the invention a plurality of pairs of invaginated parts of the wall of the lumen, vessel or organ are formed, and preferably, are formed spaced apart along a line, along which the portions thereof are to be sutured together.

In one embodiment of the invention the invaginated parts of each pair thereof are sutured together by a corresponding suture.

In another embodiment of the invention each suture comprises a suture thread terminating at its opposite ends in respective anchor elements, and defining a loop formed by two legs of the thread extending from the anchor elements, with the loop extending through a cinch clip, the cinch clip being urgeable along the loop towards the anchor elements for reducing the effective length of the suture thread extending between the anchor elements.

In another embodiment of the invention the suturing instrument comprises the suture therein, and the method further comprises:
urging a suturing needle of the suturing instrument distally through one of the invaginated parts of a pair thereof,
operating the suturing instrument to discharge one of the anchor elements of the suture on a distal side of the invaginated part through which the suturing needle extends,
withdrawing the suturing needle from the invaginated part with the thread of the suture extending from the corresponding anchor element through the invaginated part to the suturing instrument,
urging the suturing needle of the suturing instrument distally through the other one of the invaginated parts of the pair thereof,
operating the suturing instrument to discharge the other one of the anchor elements of the suture on the distal side of the invaginated part through which the suturing needle extends, and
withdrawing the suturing needle through the invaginated part with the suture extending from the corresponding anchor element through the invaginated part.

In one embodiment of the invention the suturing instrument comprises the suturing instrument according to the invention, and the suturing needle comprises the cannula of the suturing instrument according to the invention with the suture accommodating bore of the cannula charged with a plurality of the sutures.

Preferably, the sutures are sequentially inserted in the invaginated parts of the pairs thereof.

In one embodiment of the invention each suture is inserted into the corresponding pair of the invaginated parts by the suturing instrument by sequentially urging the distal piercing tip of the cannula through the invaginated parts of the pair thereof,
sequentially urging the pusher element of the suturing instrument distally in the suture accommodating bore of the cannula to discharge the distal most one of the anchor elements of the distal most suture in the suture accommodating bore of the cannula through the distal piercing tip of the cannula to deposit the anchor element on a distal side of the corresponding invaginated part of the pair thereof, and
withdrawing the cannula from the corresponding invaginated part of the pair thereof with the suture thread extending from the corresponding anchor element through the corresponding invaginated part of the pair thereof.

In another embodiment of the invention the pusher element is urged distally through the suture accommodating bore of the cannula, the predefined distance corresponding to the length of the corresponding one of the anchor elements being urged from the suture accommodating bore.

In another embodiment of the invention the cinch clip of the distal most one of the sutures in the suture accommodating bore of the cannula of the suturing instrument is discharged from the suture accommodating bore through the distal piercing tip of the cannula in the sequence in which the cinch clip is located with the anchor elements of the distal most one of the sutures in the suture accommodating bore. Preferably, the cinch clip is urged from the suture accommodating bore by urging the pusher element distally in the suture accommodating bore.

Preferably, the pusher element is urged distally in the suture accommodating bore through the predefined distance corresponding to the length of the cinch clip for urging the cinch clip from the suture accommodating bore.

In one embodiment of the invention tightening of sutures commences when the sutures have been inserted in some or all of the pairs of the invaginated parts of the portions of the lumen, vessel or organ, and preferably, the sutures are tightened when the sutures have been inserted in all of the pairs of the invaginated parts of the portions of the lumen, vessel or organ to be sutured together.

Preferably, the sutures are sequentially tightened.

In an alternative embodiment of the invention the sutures are simultaneously tightened.

In a further alternative embodiment of the invention each suture is tightened when the suture has been inserted into the corresponding pair of the invaginated parts of the portions of the lumen, vessel or organ to be sutured together.

In another embodiment of the invention each suture is tightened by urging the cinch clip along the loop of the suture thread thereof towards the anchor elements for progressively reducing the effective length of the suture thread extending between the anchor elements thereof.

In one embodiment of the invention the sutures are tightened by a tightening instrument comprising an engagement means for engaging the loop of each suture, urging the loop engaged by the engagement means to urge the cinch clip of the suture against a distal abutment face of an abutment member of the tightening instrument, and urging the loop through the cinch clip to urge the cinch clip along the loop towards the anchor elements of the suture.

In another embodiment of the invention the suturing instrument comprises the tightening instrument.

In an alternative embodiment of the invention the tightening instrument comprises a separate instrument to that of the suturing instrument.

In on embodiment of the invention the suturing is carried out internally in the interior of the lumen, vessel or organ, and the suturing instrument is entered into the interior of the lumen, vessel or organ, and preferably, the invaginated parts extend internally into the interior of the lumen, vessel or organ.

In an alternative embodiment of the invention the suturing is carried out externally of the lumen, vessel or organ internally in a cavity in which the lumen, vessel or organ is located, and the suturing instrument is entered into the cavity, and preferably, the invaginated parts extend from the lumen, vessel or organ into the cavity.

In another embodiment of the invention each invaginated part of each pair thereof is retained invaginated by a band extending tightly around the invaginated part which preferably, extends around the invaginated part adjacent the root thereof, and preferably, each band comprises an elasticated band.

In another embodiment of the invention the parts to be invaginated are invaginated by an invaginating device, which preferably, sucks the parts to be invaginated into a suction chamber thereof, and preferably, each invaginated part is retained in the suction chamber while the band is being placed on the invaginated part.

In another embodiment of the invention the invaginating device comprises a multiband ligator.

In a further embodiment of the invention the suturing instrument is entered into the lumen, vessel, organ or cavity through an access device, and preferably, the suturing instrument is entered into the lumen, vessel, organ or cavity through an instrument channel of the access device.

Alternatively, the suturing instrument is entered into the lumen, vessel, organ or cavity with the suturing instrument extending longitudinally along an outer side of the access device.

In one embodiment of the invention the suturing instrument is secured to the outer side of the access device, and preferably, is releasably secured to the outer side of the access device.

In another embodiment of the invention the suturing instrument is secured to the outer side of the access device by a plurality of clips spaced apart longitudinally along the suturing instrument and the access device.

In another embodiment of the invention the clips are configured to releasably engage the suturing instrument, and in another embodiment of the invention the clips are configured to releasably engage the access device. Advantageously, the clips are configured to releasably engage both the suturing instrument and the access device.

In another embodiment of the invention the invaginating device is entered into the lumen, vessel, organ or cavity through the access device.

In another embodiment of the invention the tightening instrument is entered into the lumen, vessel, organ or cavity through the access device.

In one embodiment of the invention the access device comprises an endoscope, and in another embodiment of the invention the access device comprises a laparoscope.

In a further embodiment of the invention the organ in which the portions thereof are to be sutured together, comprises a stomach, and preferably, the front and side walls of the stomach are to be sutured together.

In one embodiment of the invention the suturing of the stomach is carried out internally in the interior of the stomach, and preferably, the invaginated parts of the stomach extend from the front and rear walls thereof internally into the interior of the stomach. Preferably, the invaginated parts are located in pairs spaced apart along a line, along which the front and rear walls of the stomach are to be sutured, and advantageously, the invaginated parts of each pair thereof extend respectively from the front and rear walls of the stomach.

In one embodiment of the invention the suturing of the front and rear walls of the stomach together is carried out in a sleeve gastroplasty procedure.

Preferably, the sleeve gastroplasty procedure comprises an endoscopic sleeve gastroplasty procedure.

Further the invention provides an invaginating device for invaginating tissue and/or a wall of lumen, vessel or organ, in the body of the human subject, the invaginating device comprising a body member extending from a proximal end thereof to a distal end thereof, the body member defining a main axis extending from the proximal end to the distal end and comprising a connector adjacent the proximal end thereof for connecting the body member to an access device adjacent the distal end of the access device, the body member having a suction chamber extending into the body member from the distal end thereof, and an access opening to the suction chamber adjacent the distal end of the body member and facing from the body member in an axial direction distally from the distal end of the body member, the suction chamber communicating with at least one instrument channel of the access device through the connector or with an elongated tubular member extending longitudinally along the access device.

In one embodiment of the invention the suction chamber communicates with two instrument channels of the access device.

In another embodiment of the invention the body member defines a distal end face extending transversely of the main axis defined by the body member, and preferably, the access opening is formed in the distal end face of the body member.

In one embodiment of the invention the access opening to the suction chamber defines a plane extending substantially perpendicularly relative to the main axis defined by the body member.

In another embodiment of the invention the suction chamber communicates with the connector, and preferably, the suction chamber communicates directly with the connector.

In one embodiment of the invention the connector comprises a socket extending axially into the body member from the proximal end thereof, and preferably, the socket of the connector extends axially into the body member.

In one embodiment of the invention the socket defines a central axis extending substantially parallel to the main axis of the body member, and advantageously, the central axis defined by the socket substantially coincides with the main axis of the body member.

In one embodiment of the invention the suction chamber is formed by a bore extending axially into the body member from the distal end thereof, and advantageously, the bore defining the suction chamber defines a central axis, and advantageously, the central axis defined by the bore forming the suction chamber extends parallel to the main axis defined by the body member, and advantageously, the central axis defined by the bore defining the suction chamber substantially coincides with the main axis defined by the body member.

In one embodiment of the invention the bore defining the suction chamber terminates in the bore defining the socket of the connector.

In another embodiment of the invention the body member defines with a distal end of an access device to which the body member is connected, the suction chamber.

In another embodiment of the invention the suction chamber communicates with the instrument channel of the access device for applying suction to the suction chamber.

In another embodiment of the invention the suction chamber communicates with the instrument channel of the access device for receiving the suturing instrument into the suction chamber.

In another embodiment of the invention the suction chamber communicates with one of the instrument channels of the access device for applying suction to the suction chamber, and the suction chamber cooperates with the other one of the instrument channels of the access device for receiving the suturing instrument therethrough into the suction chamber.

In a further embodiment of the invention the suction chamber communicates with a single instrument channel of the access device for applying suction to the suction chamber and receiving the suturing instrument therethrough into the suction chamber.

In another embodiment of the invention the suction chamber is adapted to communicate with a suction source through a conduit or a tubular member secured to and extending longitudinally along and externally of the access device.

In another embodiment of the invention the conduit or the tubular member is releasably secured to the access device.

In another embodiment of the invention the suction chamber is configured to receive the suturing instrument secured to and extending longitudinally along and externally of the access device. Preferably, the suturing instrument is releasably secured to the access device.

The invention also provides a combination of the access device and the invaginating device coupled to the access device adjacent the distal end thereof. Preferably, the invaginating device is releasably coupled to the access device.

In another embodiment of the invention one of a conduit for applying suction to the suction chamber and a suturing instrument for extending into the suction chamber is secured to the access device and extends longitudinally along and externally of the access device.

Preferably, the one of the conduit and the suturing instrument is releasably secured to the access device, and preferably, is releasably secured to the access device, and advantageously, is secured to the access device by a plurality of clips longitudinally spaced apart along the access device.

In one embodiment of the invention the access device comprises a single channel access device, and in an alternative embodiment of the invention the access device comprises a dual channel access device.

In another embodiment of the invention the access device comprises an endoscope.

Further the invention provides an invaginating device for invaginating tissue and/or a portion of a wall of a lumen, vessel or organ in the body of a human or animal subject, the device comprising a body member, the body member comprising:
a suction chamber,
an access opening to the suction chamber for accommodating the tissue and/or the portion of the wall of the lumen, vessel or organ therethrough into the suction chamber in response to a vacuum being applied to the suction chamber for invaginating the tissue or the part of the wall of the lumen, vessel or organ,
a main communicating opening communicating with the suction chamber for communicating the suction chamber with an access device, and
a connector adapted for coupling the body member to an access device adjacent a distal end of the access device with the suction chamber communicating with the access device through the main communicating opening.

In one embodiment of the invention the suction chamber defines a central axis extending therethrough.

In another embodiment of the invention the access opening defines a plane extending perpendicularly to the central axis defined by the suction chamber.

In another embodiment of the invention the connector defines a central axis, and preferably, the connector is adapted for engaging the distal end of the access device with the central axis defined by the connector substantially coinciding with a longitudinally extending central axis defined by the access device adjacent the distal end thereof.

In another embodiment of the invention the central axis defined by the suction chamber extends substantially parallel to the central axis defined by the connector.

In one embodiment of the invention the central axis defined by the suction chamber substantially coincides with the central axis defined by the connector.

In an alternative embodiment of the invention the central axis defined by the suction chamber is offset from the central axis defined by the connector.

In another embodiment of the invention the suction chamber is of substantially circular transverse cross-section.

In another embodiment of the invention the body member comprises a side wall extending distally from the body member and around the suction chamber, and the body member and the side wall define the suction chamber.

In another embodiment of the invention the side wall terminates in a distal end defining the access opening to the suction chamber.

Preferably, the distal end of the side wall defines a distal end face adapted to engage the wall of the lumen, vessel or organ around the portion thereof to be invaginated.

In another embodiment of the invention the main communicating opening is formed by a communicating duct extending through the body member from the suction chamber to the connector.

Preferably, the connector defines a main socket for engaging the distal end of the access device therein.

Preferably, the main socket defined by the connector is of circular transverse cross-section.

In one embodiment of the invention the main socket defined by the connector is adapted to be engageable with the distal end of the access device with the central axis defined by the suction chamber substantially aligned with an instrument channel of the access device, and preferably, with the central axis defined by the suction chamber substantially coinciding with a central axis defined by the instrument channel adjacent the distal end of the instrument channel of the access device.

In another embodiment of the invention the body member defines a secondary communicating opening communicating with the suction chamber and adapted to communicate with a tubular member extending externally along the access device.

In another embodiment of the invention the secondary communicating opening defines a central axis adapted to coincide with a central axis defined by the tubular member adjacent the distal end thereof.

In one embodiment of the invention the central axis defined by the secondary communicating opening extends parallel to the central axis defined by the suction chamber, and preferably, the central axis defined by the secondary communicating opening is offset from the central axis defined by the suction chamber.

In another embodiment of the invention the central axis defined by the secondary communicating opening extends parallel to the central axis defined by the connector.

In another embodiment of the invention the central axis defined by the secondary communicating opening is offset from the central axis defined by the connector.

In one embodiment of the invention the secondary communicating opening is configured for applying a vacuum to the suction chamber.

In another embodiment of the invention the main communicating opening is configured for applying a vacuum to the suction chamber.

In a further embodiment of the invention the secondary communicating opening is adapted for delivering insufflating gas into the lumen, vessel or organ.

In a further embodiment of the invention the main communicating opening is configured for accommodating an instrument into the suction chamber, and preferably, for accommodating a suturing instrument into the suction chamber.

In another embodiment of the invention the secondary communicating opening is adapted for accommodating an instrument into the suction chamber, and preferably, for accommodating a suturing instrument into the suction chamber.

In a further embodiment of the invention the main communicating opening is adapted for accommodating a grab and hold instrument into the suction chamber, and in one embodiment of the invention the grab and hold instrument is adapted for grabbing and holding an invaginated part of the tissue and/or wall of the lumen, vessel or organ in the suction chamber.

In another embodiment of the invention the secondary communicating opening is adapted for accommodating the grab and hold instrument into the suction chamber.

In another embodiment of the invention the connector is adapted for engaging the access device adjacent the distal end thereof with the central axis defined by the suction chamber substantially aligned with an instrument channel of the access device.

In another embodiment of the invention the connector is adapted for coupling the body member to the distal end of the access device with the central axis defined by the suction chamber substantially coinciding with an instrument channel of the access device in an access device in which the instrument channel thereof is offset from a longitudinally extending central axis defined by the access device.

In one embodiment of the invention the connector is adapted for engaging the distal end of an endoscope.

### Advantages of the Invention

The advantages of the invention are many. The method for suturing two interior portions of a lumen, vessel or organ provide particularly simple and easy methods whereby two portions of a lumen, vessel or organ can be sutured together interiorly in the lumen, vessel or organ. By invaginating the parts at the locations of the portions to be sutured together in the lumen, vessel or organ prior to suturing, and then inserting the sutures into the respective pairs of the invaginated parts, the suturing of the portions of the lumen, vessel or organ together becomes a relatively simple procedure, and in particular, the suturing procedure is significantly simplified.

The method according to the invention whereby the invaginated parts may be formed at all the locations in the lumen, vessel or organ which are to be secured together prior to any suturing of the invaginated parts, provides a particularly important advantage, in that it enables the locations within the lumen, vessel or organ which are to be secured together to be accurately sutured together. By forming all the invaginated parts before any suturing is carried out enables the invaginated parts to be accurately formed at the locations which are to be sutured together. Therefore, once the invaginated parts have been accurately formed at the locations to be sutured together, the suturing of the invaginated parts is a relatively straightforward matter thereafter. Accordingly, where a series of locations in a lumen, vessel or organ are to be sutured together, for example, in the case of the formation of a gastric sleeve in an endoscopic sleeve gastroplasty procedure, the line along which the suturing is to be carried out to form the gastric sleeve can be accurately followed. This therefore ensures that suturing along the line results in a relatively straight or smoothly curved suture line, as the case may be.

Additionally, due to the fact that the corresponding invaginated parts of each pair thereof is separately sutured by a single suture, suturing of a plurality of pairs of respective locations in a lumen, vessel or organ may be carried out relatively simply and easily. Additionally, by virtue of the fact that the invaginated parts can be formed prior to suturing, additional control is provided insofar as the identification of the locations at which the invaginated parts are to be formed can be carried out with significantly greater accuracy. This, in turn results in the locations of the lumen, vessel or organ which are to be sutured together being accurately sutured together.

A particularly important advantage of the method for suturing two portions of a lumen, vessel or organ together is that by virtue of invaginating the two locations of the wall of the lumen, vessel or organ to be sutured into the lumen, vessel or organ, the cannula of the suturing instrument is passed through the invaginated parts of the wall of the lumen, vessel or organ for inserting the suture therein, without any danger of the distal piercing tip of the cannula of the suturing instrument extending through the wall of the lumen, vessel or organ to the exterior of the lumen, vessel or organ, thereby avoiding the risk of the distal piercing tip piercing or puncturing another lumen, vessel or organ exteriorly of the lumen, vessel or organ within which the suturing is being carried out.

Another particularly important advantage of the method according to the invention is that the endoscope need only be entered once into the lumen, vessel or organ, and thereafter as each suture is inserted in the corresponding pair of invaginated parts of the lumen, vessel or organ, only the suturing instrument need be withdrawn for recharging with the next suture. In the case of the suturing instrument in which a plurality of sutures may be carried in the suturing instrument, there should be no need to withdraw the suturing instrument until all the sutures in the suture accommodating bore have been inserted in the respective pairs of the invaginated parts of the lumen, vessel or organ.

An additional advantage of the invention is achieved in the embodiments of the invention in which the invaginating device and the cannula of the suturing instrument extend axially through the lumen, vessel or organ during both invaginating the invaginated part and inserting the suture therein, in other words, where the invaginating device and the cannula of the suturing instrument extend parallel to the axis of the endoscope, and therefore parallel to the portion of the wall of the lumen, vessel or organ to be sutured during invaginating and suturing. This enables both invaginating of the portion to be invaginated and insertion of the suture to be more easily carried out.

The advantage of the suturing device according to the invention is that it enables both invaginating of the invaginated parts of the lumen, vessel or organ and the insertion of the sutures into the invaginated parts with minimal withdrawing and re-entering of the access device out of and into the lumen, vessel or organ during the suturing procedure.

### BRIEF DESCRIPTION OF THE DRAWINGS

The invention will be more clearly understood from the following description of some preferred embodiments thereof which are given by way of example only with reference to the accompanying drawings, in which:

FIG. 1 is a perspective view of an invaginating device according to the invention,

FIG. 2 is another perspective view of the invaginating device of FIG. 1,

FIG. 3 is another perspective view of the invaginating device of FIG. 1,

FIG. 4 is a top plan view of a suturing apparatus also according to the invention comprising the invaginating device of FIG. 1 and a part of a suturing instrument,

FIG. 5 is an end view of a distal end of an endoscope for use with the suturing apparatus of FIG. 4,

FIG. 6 is a perspective view of the suturing apparatus of FIG. 4 in use,

FIG. 7 is another perspective view of the suturing apparatus of FIG. 4 in use,

FIG. 8 is a perspective view of a suture for use with the suturing apparatus of FIG. 4,

FIG. 9 is a cross-sectional side elevational view of the suturing instrument of the suturing apparatus of FIG. 4,

FIG. 10 is a partly cutaway perspective view of a stomach being sutured by the suturing apparatus of FIG. 4 in an endoscopic sleeve gastroplasty procedure,

FIG. 11 is a view similar to FIG. 10 illustrating the suturing apparatus of FIG. 4 in use during the endoscopic sleeve gastroplasty procedure,

FIG. 12 is a view similar to FIG. 10 illustrating the suturing apparatus of FIG. 4 in use during the endoscopic sleeve gastroplasty procedure,

FIG. 13 is a view similar to FIG. 10 illustrating the suturing apparatus of FIG. 4 in use during the endoscopic sleeve gastroplasty procedure,

FIG. 14 is a view similar to FIG. 10 illustrating the suturing apparatus of FIG. 4 in use during the endoscopic sleeve gastroplasty procedure,

FIG. 15 is a view similar to FIG. 10 illustrating the suturing apparatus of FIG. 4 in use during the endoscopic sleeve gastroplasty procedure,

FIG. 16 is a cross-sectional side elevational view of the stomach of FIG. 10 during the endoscopic sleeve gastroplasty procedure,

FIG. 17 is a cross-sectional end elevational view of the stomach of FIG. 10 on completion of the endoscopic sleeve gastroplasty procedure,

FIGS. 18 to 23 are diagrammatic views illustrating the steps carried out in the endoscopic sleeve gastroplasty procedure by the suturing apparatus of FIG. 4,

FIG. 24 is a perspective view of the suturing apparatus of FIG. 4 during another step of the endoscopic sleeve gastroplasty procedure,

FIGS. 25 to 29 are diagrammatic views illustrating the steps being carried out in a method according to another embodiment of the invention for carrying out an endoscopic sleeve gastroplasty procedure using a multiband ligator and the suturing instrument of FIG. 9,

FIG. 30 is a view of a portion of an internal wall of a lumen, vessel or organ of a subject in which opposite edge portions of a weakened region of the wall of the lumen, vessel or organ are in the process of being sutured together by a method similar to that described with reference to FIGS. 25 to 39,

FIG. 31 is a cross-sectional side elevational view of a suturing instrument according to the invention illustrating a part of the suturing instrument in block representation,

FIG. 32 is a cross-sectional side elevational view of a portion of the suturing instrument of FIG. 31 illustrated in a different state to that of FIG. 31,

FIG. 33 is a perspective view of a portion of the suturing instrument of FIG. 31,

FIG. 34 is a perspective view of an enlarged part of the portion of FIG. 33 of the suturing instrument of FIG. 31,

FIG. 35 is an enlarged side elevational view of a part of the portion of FIG. 33 of the suturing instrument of FIG. 31,

FIG. 36 is an angular transverse cross-sectional view on the line XXXVI-XXXVI of FIG. 35 following one of a slot section of an interrupted helical slot of the part of FIG. 35,

FIG. 37 is an enlarged view of a detail of the portion of FIG. 33 of the suturing instrument of FIG. 31,

FIG. 38 is a perspective view of a suturing apparatus according to another embodiment of the invention comprising an invaginating device also according to the invention secured to an endoscope, and the suturing instrument of FIG. 31 releasably secured to the endoscope,

FIG. 39 is a side elevational view of the suturing apparatus of FIG. 38,

FIG. 40 is a perspective view of the invaginating device of FIG. 38,

FIG. 41 is an end elevational view of the invaginating device of FIG. 38,

FIG. 42 is a view similar to FIG. 38 illustrating the invaginating device of FIG. 38 in use,

FIG. 43 is a side elevational view of the invaginating device of FIG. 38 also in use,

FIG. 44 is a side elevational view of the invaginating device of FIG. 38 also in use,

FIG. 45 is a perspective view of the invaginating device of FIG. 38 in use,

FIG. 46 is an end elevational view of an endoscope with the invaginating device of FIG. 38 secured to the endoscope,

FIG. 47 is a perspective view of a combination according to the invention of an access device, in this case an endoscope, and an invaginating device according to another embodiment of the invention releasably secured to the endoscope adjacent a distal end thereof,

FIG. 48 is a cross-sectional side elevational view of the invaginating device of FIG. 47,

FIG. 49 is a cross-sectional side elevational view of the invaginating device of FIG. 47 attached to the distal end of the endoscope along with a suturing instrument,

FIG. 50 is a perspective view of the invaginating device as illustrated in FIG. 49,

FIG. 51 is a cross-sectional side elevational view of the combination of the endoscope and the invaginating device of FIG. 47 along with a suturing instrument,

FIGS. 52 to 54 are cross-sectional side elevational views of the invaginating device of FIG. 47 attached to the distal end of the endoscope of FIG. 47 illustrated in use,

FIG. 55 is a perspective view of an invaginating device according to a further embodiment of the invention illustrated secured to a distal end of an endoscope and including a suturing instrument,

FIG. 56 is an end elevational view of the invaginating device of FIG. 55 also illustrated secured to the distal end of the endoscope,

FIG. 57 is a perspective view of a suturing apparatus according to another embodiment of the invention comprising an invaginating device also according to another embodiment of the invention illustrated secured to a distal end of an endoscope,

FIG. 58 is another perspective view of the suturing apparatus of FIG. 57 illustrated secured to the distal end of the endoscope, and

FIG. 59 is a perspective view of an invaginating device according to another embodiment of the invention.

### DETAILED DESCRIPTION OF THE INVENTION

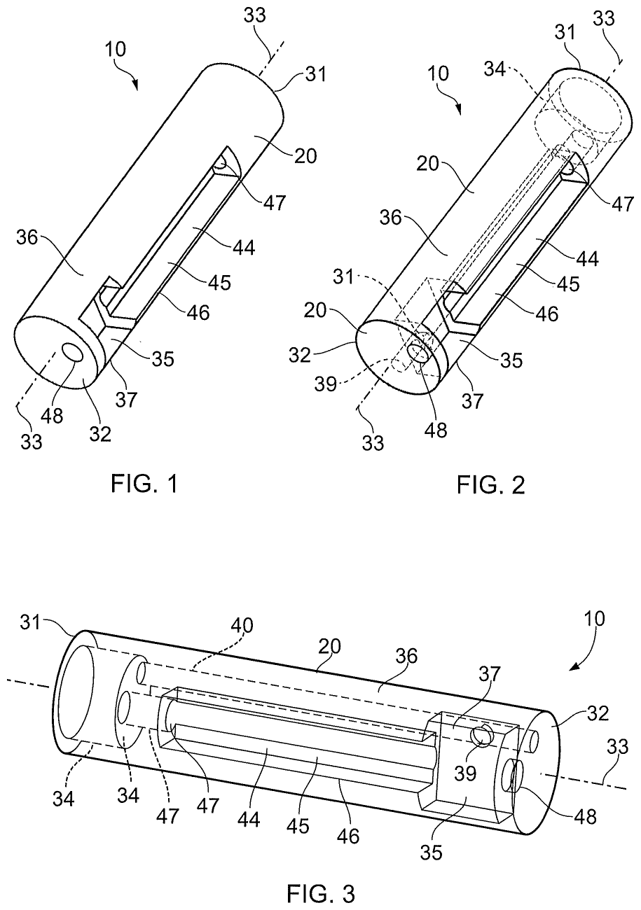

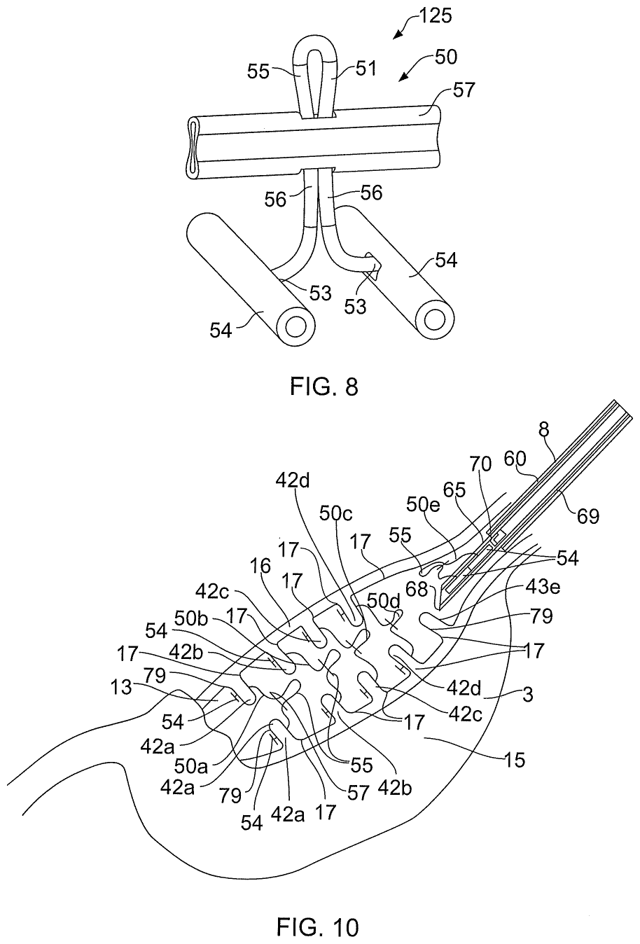

Referring to the drawings, and initially to FIGS. 1 to 24, there is illustrated a suturing apparatus according to the invention indicated generally by the reference numeral 1 for endoscopically suturing an interior part of a lumen, vessel or organ, in this embodiment of the invention a stomach 3, in order to carry out an endoscopic sleeve gastroplasty procedure internally in the interior 5 of the stomach 3 of a human or animal subject to form a gastric sleeve 6 to thereby reduce the volume of the stomach 3, as will be described below. The suturing apparatus 1 comprises a suturing instrument indicated generally by the reference numeral 8, see FIGS. 9 to 15 and an invaginating device which is also according to the invention and is indicated generally by the reference numeral 10, see FIGS. 1 to 7. During the endoscopic sleeve gastroplasty procedure, inner sides 13 of opposite walls of the stomach 3, namely, a front wall 15 and a rear wall 16 of the stomach 3 are sutured together at a plurality of spaced apart locations 17 by sutures 50, see FIG. 8 along a line 19 extending substantially longitudinally along the stomach 3 to form the sleeve 6, see FIG. 16.

Turning initially to the invaginating device 10, the invaginating device 10 comprises a body member, namely, an elongated body member 20 of a transparent material, which may be of any suitable material, for example, a plastics material, a polymer material, such as a silicone rubber material, and ideally, should be of a flexible bendable material, and may be of a flexible bendable plastic thermoplastic elastomer, such as Pebax. The body member 20 is adapted for coupling to a distal end of an access device, in this case a distal end 22 of a dual channel endoscope 24, a distal portion 25 only of which is illustrated. Such dual channel endoscopes as the endoscope 24 will be well known to those skilled in the art, and only the aspects of the endoscope 24, which are relevant to the invaginating device 10 will be described.

Briefly, a pair of instrument channels 26 and 27 extend through the endoscope 24 for accommodating instruments therethrough. A vision channel 28 extends through the endoscope 24 for facilitating viewing of the interior of the lumen, vessel or organ into which the distal end 22 of the endoscope 24 extends, which in this case, is the stomach 3. The vision channel 28 terminates in a lens 29 adjacent the distal end 22 of the endoscope 24. A pair of light channels 30 extend through the endoscope 24 for illuminating the lumen, vessel or organ into which the distal end 22 of the endoscope 24 extends, which in this case is the stomach 3.

The body member 20 of the invaginating device 1 is of cylindrical shape having an outer cylindrical surface 36 and extends from a proximal end 31 to a distal end 32, and defines a longitudinal main central axis 33 extending distally from the proximal end 31 to the distal end 32. A connector for connecting the invaginating device 10 to the distal end 22 of the endoscope 24 in this embodiment of the invention comprises a socket 34 formed into the body member 20 from the proximal end 31. The socket 34 is configured so that when the body member 20 is secured to the distal end 22 of the endoscope 24, the main axis 33 of the body member 20 extends parallel to, and in this embodiment of the invention, substantially coincides with a central longitudinally extending axis 38 of the endoscope 24 adjacent the distal end 22 thereof.

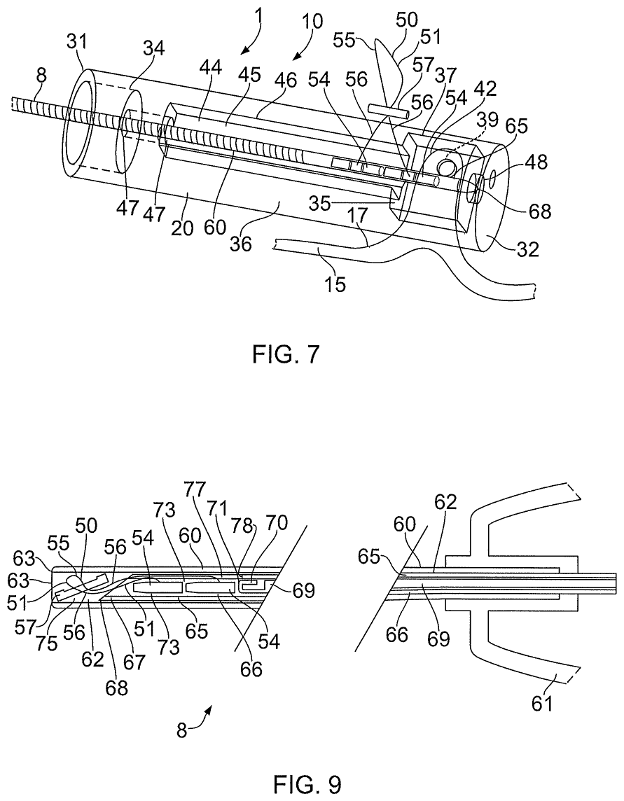

A suction chamber 35 extends transversely into the body member 20 intermediate the proximal end 31 and the distal end 32 thereof and towards the distal end 32, and defines an access opening 37 in the cylindrical surface 36. The access opening 37 communicates with the suction chamber 35 and faces in a direction transversely of the main central axis 33 of the body member 20. A communicating duct 39 formed by an elongated suction bore 40 extends through the body member 20 from the socket 34 and terminates in a suction port 41 in the suction chamber 35 for communicating the suction chamber 35 with the instrument channel 27 of the endoscope 24 for applying suction to the suction chamber 35 when suction is applied to the instrument channel 27. Accordingly, when the access opening 37 of the suction chamber 35 is brought into engagement with one of the locations 17 on the inner side 13 of one of the front and rear walls 15 and 16 of the stomach 3, which is to be sutured to a corresponding location 17 of the other one of the front and rear walls 15 and 16 thereof, and suction is applied to the suction chamber 35, a part 42 of the stomach wall at the location 17 is invaginated by being sucked into the suction chamber 35 through the access opening 37 to thereby form an invaginated part 42, see FIG. 7. The suction bore 40 is closed adjacent the distal end 32 of the body member 20.

An instrument passageway 44 extends through the body member 20 from the socket 34 into the suction chamber 35 for guiding the suturing instrument 8 therethrough to the suction chamber 35 for suturing corresponding pairs of the locations 17 of the front and rear walls 15 and 16 together of the stomach 3 as will be described in more detail below. The instrument passageway 44 comprises a distal portion formed by a guide channel 45 which communicates with and extends proximally from the suction chamber 35. The guide channel 45 defines an open mouth 46 in the cylindrical surface 36, which extends proximally from the access opening 37, and faces in the same direction as the access opening 37 faces. A proximal portion of the instrument passageway 44 is formed by a proximal guide bore 47, which extends from the guide channel 45 into the socket 34 for communicating the guide channel 45 with the instrument channel 26 of the endoscope 24. In some embodiments of the invention the guide channel 45 of the instrument passageway 44 may be dispersed with, and the guide bore 47 would extend to the suction chamber 35. Additionally, in other embodiments of the invention the body member 20 may be configured so that the suturing instrument 8 would extend directly from the distal end of the endoscope 24 into the suction chamber 35. The instrument channel 26 of the endoscope 24 accommodates the suturing instrument to the instrument passageway 44 of the invaginating device 10. A distal instrument accommodating bore 48 aligned with the instrument passageway 44 extends distally from the suction chamber 35 and through the distal end 32 of the body member 20 for accommodating the suturing instrument 8 therethrough for a purpose to be described below.

In this embodiment of the invention by virtue of the fact that the body member 20 is of a transparent material, the body member 20 permits viewing of the interior of the stomach through the distal end 3 thereof through the vision channel 28 of the endoscope 24.

The suturing instrument 8 is adapted to sequentially suture corresponding pairs of the invaginated parts 42 formed on the front and rear walls 15 and 16 of the stomach 3 at corresponding pairs of the locations 17 together with respective sutures 50, one of each pair of the invaginated parts 42 being located on the front wall 15 of the stomach 3, and the other one of each pair of invaginated parts 42 being located on the rear wall 16 of the stomach 3.

Referring to FIG. 8 each suture 50 comprises a suture thread 51, which is secured at its opposite ends 53 in respective anchor elements 54. Legs 56 of the suture thread 51 extending from the anchor elements 54 form a loop 55 which extend through a cinch clip 57. The cinch clip 57 tightly engages the legs 56 of the suture thread 51 of each suture 50, and is provided for tightening the suture 50 as will be described below. Additionally, as will also be described in more detail below, the suturing of each pair of the corresponding invaginated parts 42 by the suturing instrument 8 with one of the sutures 50 is carried out by urging the distal end 53 of one leg 56 of the suture thread 51 of the suture 50 along with the corresponding anchor element 54 through one of the invaginated parts 42 of the pair thereof, with the corresponding anchor element 54 located on the distal side of the invaginated part 42, and with the corresponding leg 56 of the suture 50 extending through the invaginated part 42 from the corresponding anchor element 54. The distal end 53 of the other leg 56 of the suture thread 51 of the suture 50 along with the corresponding anchor element 54 is urged through the other one of the pair of the corresponding invaginated parts 42, with the corresponding anchor element 54 located on the distal side of that invaginated part 42, and with the corresponding leg 56 of the suture 50 extending through that invaginated part 42 from the corresponding anchor element 54. On the legs 56 of the suture 50 being inserted through the respective invaginated parts 42, the suture 50 is tightened in order to draw the invaginated parts 42 together. To tighten the suture 50, the cinch clip 57 is urged along the loop 55 towards the anchor elements 54 for reducing the effective length of the suture thread 51 of the suture 50 between the anchor elements 54, for in turn drawing the invaginated parts 42 together.

The suturing of each pair of the corresponding invaginated parts 42 of the front and rear walls 15 and 16 of the stomach 3 with the suturing instrument 8 and the invaginating device 10 of the suturing apparatus 1 will now be described.

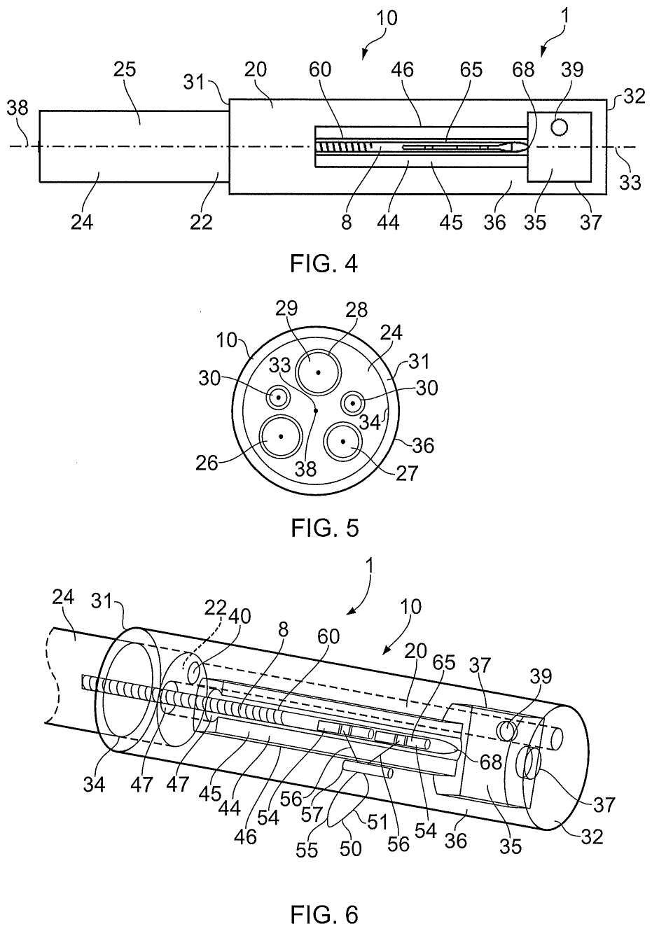

Referring now to FIGS. 4 to 24, in this embodiment of the invention the suturing instrument 8 comprises a suturing instrument substantially similar to the suturing instrument disclosed in the applicant's PCT Specification No. WO 2021/165945. The suturing instrument 8, see in particular FIG. 9, comprises an elongated flexible tubular outer sleeve 60 which extends from a proximal operating handle, only a portion 61 of which is illustrated, and which is located externally of the proximal end (not shown) of the endoscope 24. The outer sleeve 60 extends through the instrument channel 26 of the endoscope 24 and in turn into the instrument passageway 44 of the invaginating device 10. The outer sleeve 60 is of circular transverse cross-section having a cannula accommodating bore 62 of circular transverse cross-section extending therethrough and terminates in a distal end 63, which defines a distal annular abutment face 64 for a purpose to be described below. A suturing needle comprising an elongated cannula 65 also of circular transverse cross-section is slideable longitudinally in the cannula accommodating bore 62 of the outer sleeve 60 and terminates at its distal end 67 in a pointed distal piercing tip 68. The cannula 65 defines an elongated bore 66 of circular transverse cross section extending longitudinally therethrough. The cannula 65 extends through the outer sleeve 60 from the operating handle 61, and is urgeable by a first operating element (not shown) in the operating handle 61 distally from a withdrawn state illustrated in FIG. 9 withdrawn into the cannula accommodating bore 62, outwardly through the distal end 63 of the outer sleeve 60 for carrying out the suturing operation as will be described below.

An elongated pusher element 69 is located in the bore 66 of the cannula 65 and extends from the operating handle 61 through the bore 66 and terminates in a distal hook 70, which in turn defines a distal abutment end 71. The pusher element 69 is operable in a withdrawn state illustrated in FIG. 9 with the abutment end 71 of the hook 70 defining with a distal portion of the bore 66 of the cannula 65 an anchor element accommodating chamber 73 for accommodating the anchor elements 54 of one of the sutures 50 with the anchor elements 54 sequentially aligned in the anchor element accommodating chamber 73. The pusher element 69 is urgeable distally by a second operating element (not shown) of the operating handle 61 from the withdrawn state for sequentially discharging the anchor elements 54 of the suture 50 through the distal piercing tip 68 of the cannula 65 during suturing, as will be described below.

The distal end 67 of the cannula 65 in the withdrawn state thereof, illustrated in FIG. 9, defines with a distal portion of the cannula accommodating bore 62 of the outer sleeve 60 a cinch clip accommodating chamber 75 for accommodating the cinch clip 57 of the suture 50. A longitudinally extending suture accommodating slot 77 extending longitudinally into the cannula 65 from the distal end 67 thereof to a location 78 accommodates the legs 56 of the suture 50 therethrough from the anchor elements 54 to the cinch clip 57. The cannula 65 is urgeable distally by the first operating element (not shown) in the operating handle 61 from the withdrawn state for discharging the cinch clip 57 of the suture 50 through the distal end 63 of the sleeve 60 during suturing as will be described below.

In order to assist in an understanding of the suturing apparatus 1, the operation of the suturing instrument 8 will now be described in conjunction with the invaginating device 10 for forming and suturing pairs of the corresponding invaginated parts 42 at corresponding ones of the locations 17 on the inner sides 13 of the respective front and rear walls 15 and 16 of the stomach 3.

Referring now to FIGS. 9 to 24, initially, with the cannula 65 and the pusher element 69 of the suturing instrument 8 in the withdrawn states, the suturing instrument 8 is charged with one of the sutures 50, with the anchoring elements 54 aligned sequentially in the anchor element accommodating chamber 73 and with the cinch clip 57 located in the cinch clip accommodating chamber 75. The invaginating device 10 is secured to the distal end 22 of the endoscope 24 by engaging the distal end 22 of the endoscope 24 in the socket 34 of the body member 20 with the guide bore 47 of the instrument passageway 44 of the body member 20 aligned with and communicating with the instrument channel 26 of the endoscope 24, and the suction bore 40 of the body member 20 aligned with and communicating with the instrument channel 27 of the endoscope 24.

The endoscope 24 with the invaginating device 10 secured thereto is urged orally through the oesophagus into the stomach 3 of the subject until the invaginating device 10 is located in the stomach 3. The stomach 3 is insufflated with carbon dioxide gas through any suitable one of the channels of the endoscope 24.

The suturing instrument 8 is then urged through the instrument channel 26 of the endoscope 24, and in turn into the instrument channel 44 of the invaginating device 10. The suturing instrument 8 is entered into the endoscope 24 by urging the outer sleeve 60 of the suturing instrument 8 through the instrument channel 26 of the endoscope 24, and in turn urging the outer sleeve 60 of the suturing instrument 8 into the guide channel 45 of the invaginating device 10 with the distal end 61 of the outer sleeve 60 terminating in the guide channel 45 towards the suction chamber 35 but spaced apart therefrom.

The endoscope 24 is maneuvered in order to orient the invaginating device 10 in the stomach 3 to align the suction chamber 35 with the first of the locations 17 of a pair of the locations 17 to be sutured together, see FIG. 18. The access opening 37 to the suction chamber 35 is brought into engagement with the appropriate one of the locations 17 of the inner side 13 of one of the front or rear walls 15 or 16 of the stomach 3. Suction is then applied to the suction chamber 35 through the instrument channel 27 for drawing a portion of the front or rear walls 15 or 16 adjacent the location 17 into the suction chamber 35 for forming the invaginated part 42, see FIG. 19. With the suction maintained in the suction chamber 35, the invaginated part 42 of the location 17 is retained within the suction chamber 35.

The cannula 65 of the suturing instrument 8 is urged distally in the outer sleeve 60 from the withdrawn state by the first operating element (not shown) of the operating handle 61 for urging the cinch clip 57 out of the cinch clip accommodating chamber 75 and into the stomach 3, see FIGS. 6 and 10. However, in FIG. 10 and FIGS. 11 to 15 the invaginating device 10 has been omitted for clarity, so that the operation of the suturing instrument 8 can be clearly illustrated during the suturing process. Additionally, in FIGS. 10 to 15 the sutures 50 are illustrated inserted into four of the pairs of the corresponding invaginated parts 42a to 42d from the distal most pair of invaginated parts 42a. One of the sutures 50 has not yet been inserted into the proximal most pair of the invaginated parts 42e, and the insertion of one of the sutures 50 into the proximal most pair of the invaginated parts 42e by the suturing instrument 8 will be described with reference to FIGS. 10 to 15 and FIGS. 20 to 23.

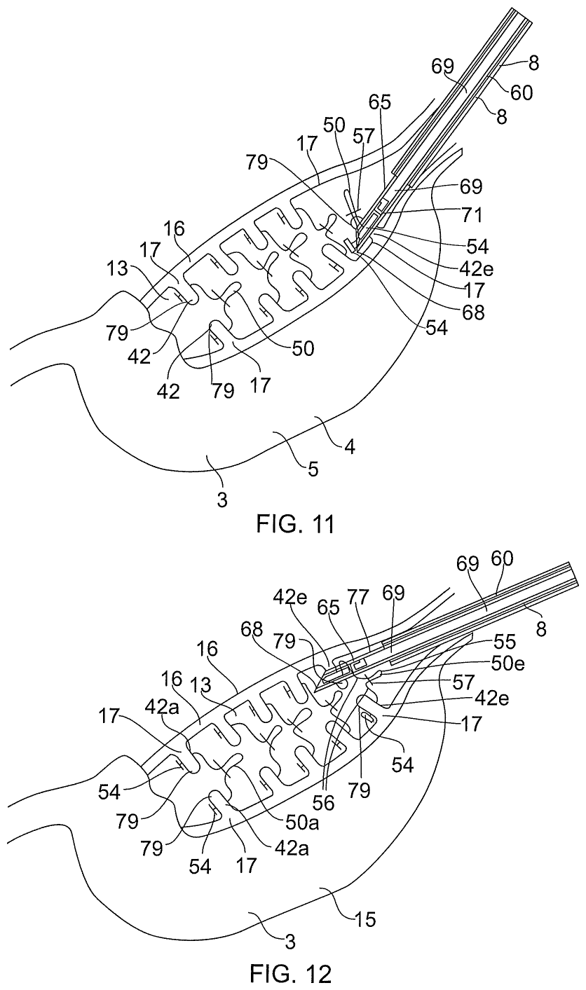

Once the cinch clip 57 has been discharged from the cinch clip accommodating chamber 75, the cannula 65 is then urged further distally to extend outwardly through the distal end 63 of the outer sleeve 60 and into the suction chamber 35 to pierce completely through the invaginated part 42 retained therein by the suction applied thereto, see FIGS. 7, 11 and 20. When the distal piercing tip 68 of the cannula 65 has exited the distal side 79 of the invaginated part 42, the pusher element 69 is urged distally from its withdraw state by the second operating element (not shown) of the operating handle 61 a distance sufficient to urge the first one of the anchoring elements 54 of the suture 50 out of the anchor element accommodating chamber 73, so that the first one of the anchor elements 54 is located adjacent the distal side 79 of the invaginated part 42, see FIGS. 11 and 21. The cannula 65 is then withdrawn from the invaginated part 42, and in turn from the suction chamber 35 into the guide channel 45. At this stage the suture thread 41 of the suture 50 extends from the first one of the anchor elements 54 through the invaginated part 42, and in turn extends through the cinch clip 57, which is now loose within the stomach 3 and back to the other one of the anchor elements 54 still in the anchor element accommodating chamber 73 in the cannula 65, see FIGS. 11 and 21. The suction applied to the suction chamber 35 is terminated, thereby releasing the invaginated part 42 from the suction chamber 35 with one leg 56 of the suture 50 inserted into the invaginated part 42.

The invaginating device 10 is then rotated in the stomach 3 by the endoscope 24 to align the suction chamber 35 with the location 17 in the other one of the front and rear walls 15 and 16 which corresponds with the location 17 of the invaginated part 42 into which one of the legs 56 of the suture 50 has just been inserted. The access opening 37 of the suction chamber 35 is brought into engagement with that location 17, and suction is again applied to the suction chamber 35 through the instrument channel 27 for drawing a part 42 at that location 17 into the suction chamber 35 to form an invaginated part 42 at that location 17, see FIG. 21. With the just invaginated part 42 retained in the suction chamber 35 by the suction applied thereto, the cannula 65 of the suturing instrument 8 is again urged into the suction chamber 35, and in turn through the invaginated part 42 until the distal piercing tip 68 of the cannula 65 extends through the distal side 79 of the invaginated part 42, see FIGS. 12 and 21.

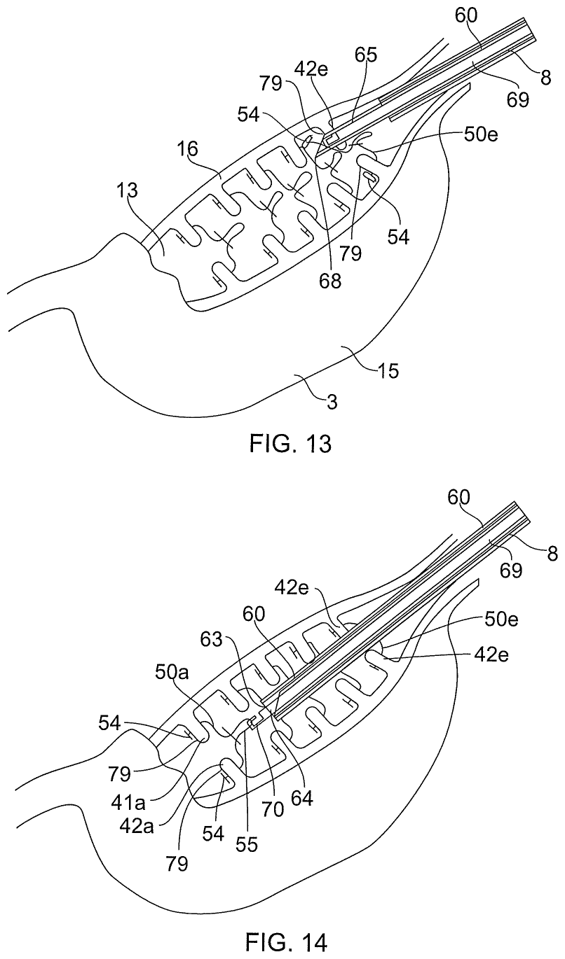

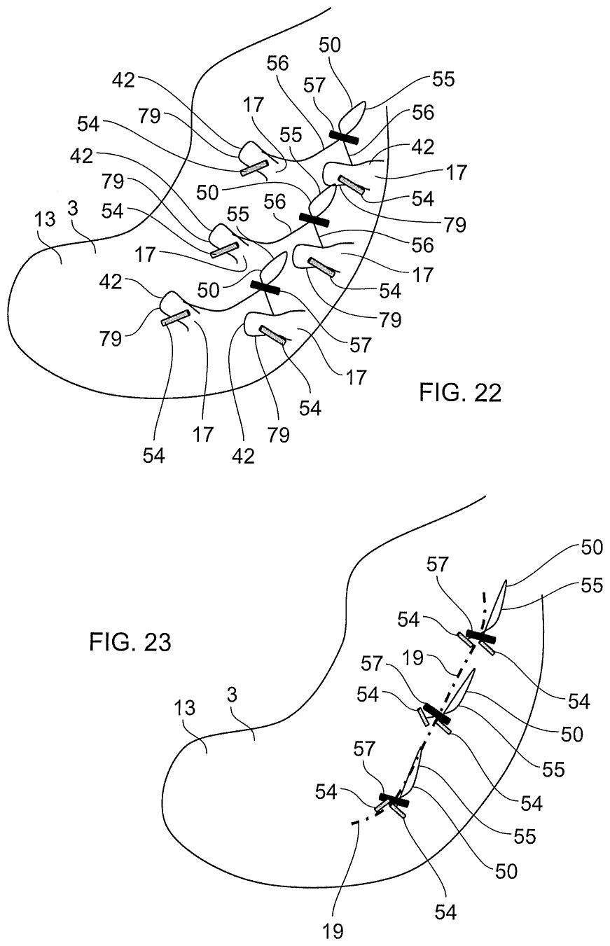

The pusher element 69 is again urged distally by the second operating element (not shown) of the operating handle 61 of the suturing instrument 8 for urging the second one of the anchor elements 54 out of the anchor element accommodating chamber 73, so that the second anchor element 54 is located on the distal side 79 of the invaginated part 42, see FIG. 13. The cannula 65 is withdrawn from the invaginated part 42 and from the suction chamber 35 into the guide channel 45. The suction applied to the suction chamber 35 is terminated, thereby releasing the invaginated part 42 from the suction chamber 35. At this stage the legs 56 of the suture thread 51 of the suture 50 extend from the respective anchor elements 54 through the respective invaginated parts 42 of the corresponding locations 17 to form the loop 55 which extends through the cinch clip 57, see FIG. 22.

Once a suture 50 has been inserted into the corresponding pair of invaginated parts 42, the suturing instrument 8 is withdrawn from the invaginating device 10 through the endoscope 24, and is recharged with the next suture 50, and returned to the invaginating device 10 through the endoscope 24. Invaginated parts 42 are then formed in the next pair of locations 17 on the inner surfaces 13 of the respective front and rear walls 15 and 16 of the stomach 3, and the suture 50 in the suturing instrument 8 is inserted in the invaginated parts 42 in a similar manner as that just described with the invaginating device 10 and the suturing instrument 8.

If at this stage, as illustrated in FIGS. 14 and 16 the invaginated parts 42 have been formed at all the pairs of locations 17 to be sutured together, and the sutures 50 have been inserted in all the invaginated parts 42, the sutures 50 are sequentially tightened from the distal most suture 50a to the proximal most suture 50e to draw the invaginated parts 42 of each pair thereof together.

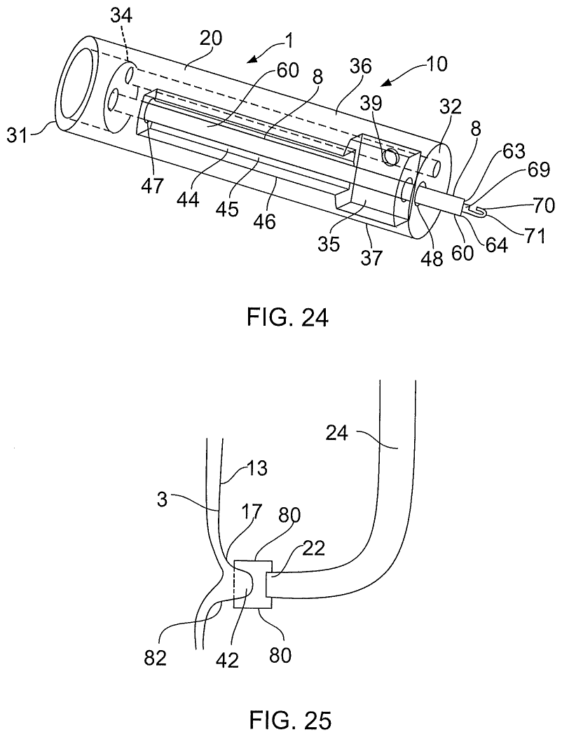

The tightening of the sutures 50 is carried out with the suturing instrument 8 as follows. The cannula 65 is withdrawn into the outer sleeve 60 of the suturing instrument 8, and the distal end 63 of the outer sleeve 60 is urged through the suction chamber 35, and in turn through the distal instrument accommodating bore 48 of the invaginating device 10, see FIG. 24. With the distal end 63 of the outer sleeve 60 of the suturing instrument 8 extending through the distal instrument accommodating bore 48 of the invaginating device 10, the outer sleeve 60 is urged distally in the stomach 3 to the loop 55 of the distal most suture 50, see FIG. 14.

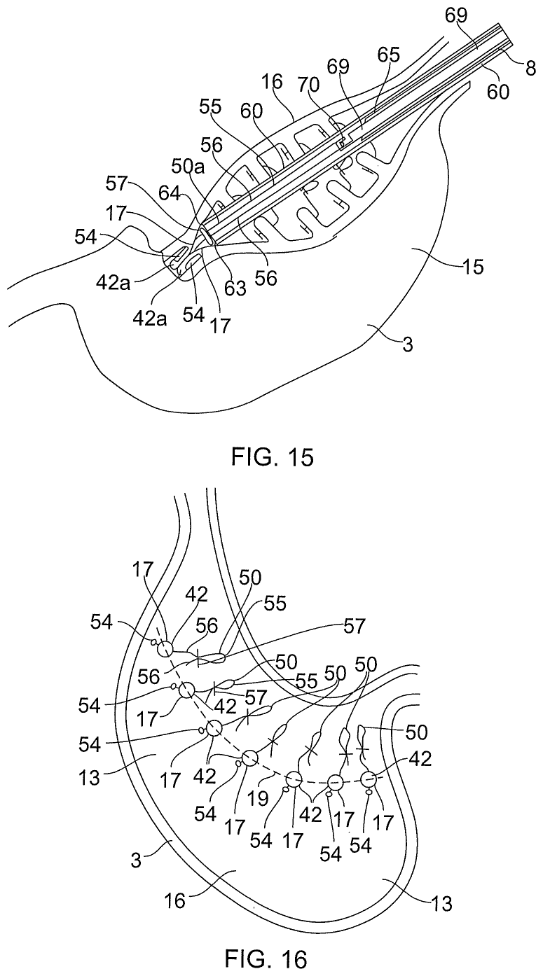

The pusher element 69 is urged distally through the cannula 65, and in turn through the distal end 63 of the outer sleeve 60 by the second operating element (not shown) of the operating handle 61 to expose the distal hook 70 at the distal end of the pusher element 69, see FIGS. 14 and 24. The hook 70 is engaged with the loop 55 of the distal most suture 50a, see FIG. 14. The pusher element 69 is then withdrawn proximally into the outer sleeve 60, and the pusher element 69 and the cannula 65 are both drawn proximally in the outer sleeve 60 by the first and second operating elements (not shown) of the operating handle 61 to in turn draw the loop 55 of the distal most suture 50a into the cannula bore 62 of the outer sleeve 60, see FIG. 15. As the pusher element 69 draws the loop 55 of the distal most suture 50a into the cannula bore 62, the cinch clip 57 engages the distal abutment face 64 at the distal end 63 of the outer sleeve 60.

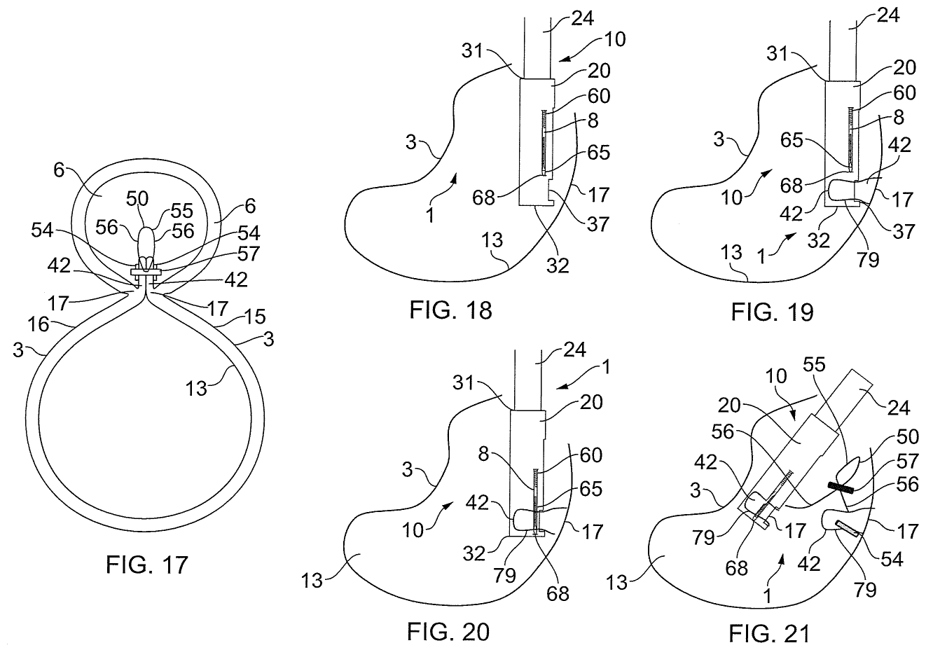

Further urging of the pusher element 69 proximally through the cannula bore 62 in the outer sleeve 60 results in the cinch clip 57 being urged along the legs 56 of the loop 55 of the suture 50a towards the anchor elements 54 of the suture 50a, for in turn progressively reducing the effective length of the suture thread 51 of the suture 50a between the anchor elements 54 thereof, see FIG. 15. By reducing the effective length of the suture thread 51 between the anchor elements 54 the two distal most invaginated parts 42a of the corresponding locations 17 of the front and rear walls 15 and 16 of the stomach 3 are drawn together, as are the inner sides 13 of the front and rear stomach walls 15 and 16, adjacent the corresponding pair of locations 17, drawn together. The pusher element 69 is urged proximally through the outer sleeve 60 until the cinch clip 57 is tightly abutting the two distal most invaginated parts 42a with the two invaginated parts 42a tightly secured together by the suture 50a, see FIGS. 15 and 17.

Once the two distal most invaginated parts 42a have been tightly secured together by the suture 50a, the pusher element 69 is urged distally in the outer sleeve 60 until the hook 70 extends through the distal end 63 thereof. The hook 70 is then disengaged from the loop 55 of the distal most suture 50a, and is engaged with the loop 55 of the next suture, namely, the suture 50b for tightening thereof, for in turn drawing the pair of the invaginated parts 42b tightly together, as already described with reference to the suture 50a and the invaginated parts 42a. And so the tightening of the sutures 50 continues sequentially from the distal most suture 50a to the proximal most suture 50e until all the sutures 50 have been tightened and the pairs of the invaginated parts 42 at the location 17 have been secured together by the sutures 50 to form the sleeve 6 in the stomach 3.

Instead of deferring tightening of the sutures 50 until the sutures 50 have been inserted into all of the pairs of the invaginated parts 42, the sutures 50 may be tightened as each suture 50 is inserted into the corresponding pair of the invaginated parts 42.

Additionally, when tightening of the sutures 50 is being deferred until the sutures have been inserted into all of the pairs of the invaginated parts, it is envisaged that prior to commencing tightening of the sutures 50, the endoscope 24 with the invaginating device 10 attached thereto may be withdrawn from the subject, and the invaginating device 10 would be detached from the endoscope 24. The endoscope 24 would then be returned orally through the oesophagus to the stomach 3. The suturing instrument 8 would then be entered through the instrument channel 26 of the endoscope 24 into the stomach 3, and the sutures 50 would then be tightened by the suturing instrument 8 as already described but without the presence of the invaginating device 10 located on the distal end 22 of the endoscope 24.

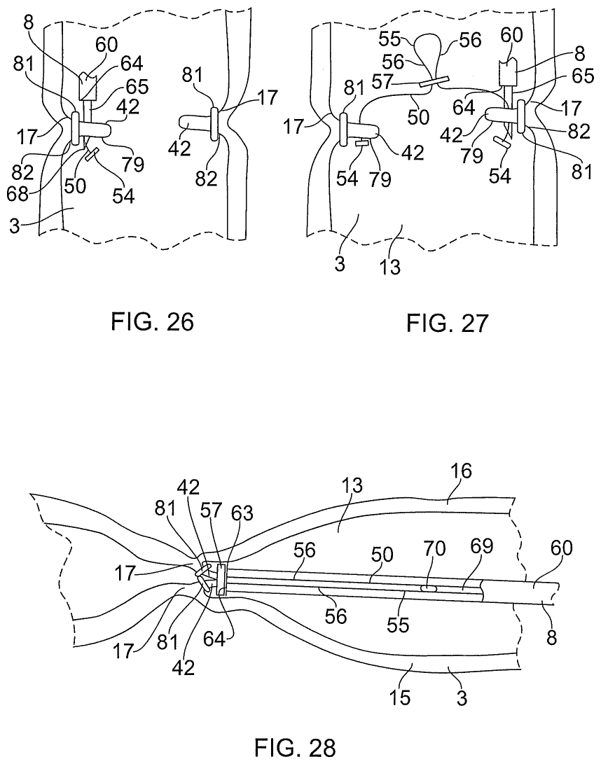

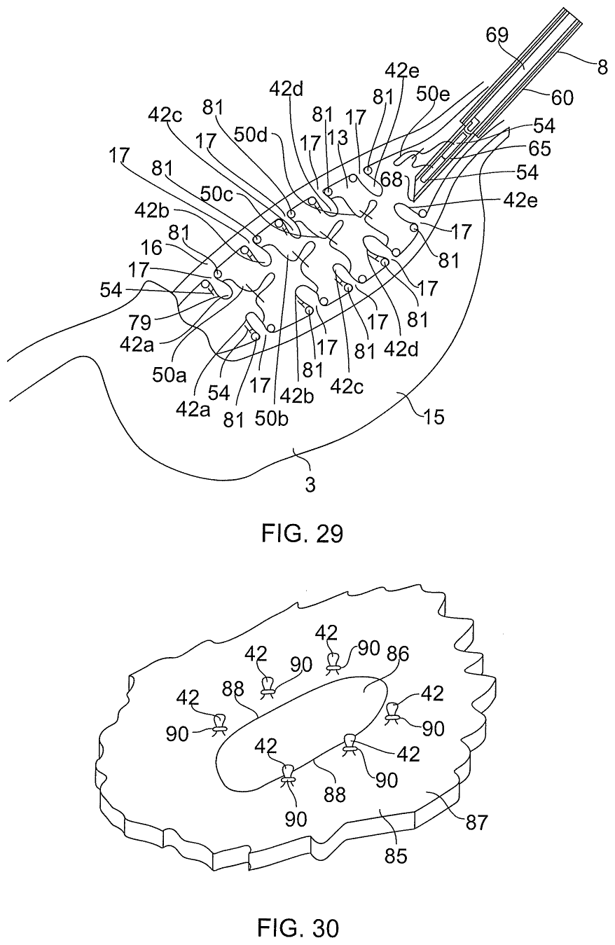

Referring now to FIGS. 25 to 29, an alternative method also according to the invention for endoscopically carrying out a sleeve gastroplasty procedure in the stomach 3 of a subject will now be described. In this embodiment of the invention the invaginating device 10 is not required. Instead of forming the invaginated parts 42 with the invaginating device 10, the invaginated parts 42 are formed using a multiband ligator 80 of the type supplied by, for example, Cooke Medical and sold under the tradename 4 6 10 Shooter Saeed Multi Band Ligator MBL, or Boston Scientific under the tradename Speedband Superview Super 7. In this case a plurality of invaginated parts 42 of the pairs thereof are formed at the respective locations 17 in the front and rear walls 15 and 16 of the stomach 3 before suturing of the invaginated parts 42 commences, and preferably, all the invaginated parts 42 are formed prior to the commencement of suturing.

The multiband ligator 80 is secured to the distal end of an endoscope, for example, the dual channel endoscope 24, although, in this embodiment of the invention a single channel endoscope would be adequate, since in this embodiment of the invention only a single instrument channel is required, see FIG. 25. The endoscope 24 with the multiband ligator 80 attached thereto is urged orally through the oesophagus into the stomach 3 of the subject. The multiband ligator 80 is sequentially urged into engagement perpendicularly with the inner side 13 of the front and rear walls 15 and 16 of the stomach 3 at respective locations 17 for forming a corresponding invaginated part 42. At each location 17 suction is applied to the multiband ligator 80 for drawing a part of the stomach wall adjacent the corresponding location 17 into the multiband ligator 80 to form the invaginated part 42. As each invaginated part 42 is formed, one of a plurality of bands 81 of elasticated material, which are located on the multiband ligator 80, is discharged from the multiband ligator 80 onto the invaginated part 42 adjacent the root 82 adjacent the one of the front and rear walls 15 and 16 of the stomach 3, at which the invaginated part 42 is being formed, for retaining the invaginated part 42 in an invaginated state.

Once the respective pairs of the invaginated parts 42 have been formed, the endoscope 24 is removed from the subject, and the multiband ligator 80 is detached from the endoscope 24. The endoscope 24 is then returned orally to the stomach 3. The suturing instrument 8 charged with a suture 50 is entered into the stomach 3 through the instrument channel 26 of the endoscope 24, and suturing of the pairs of the invaginated parts 42 together using the suturing instrument 8 is carried out as described above but without the multiband ligator 80.

In some cases when the invaginated parts 42 have been formed at all the locations 17 on the front and rear walls 15 and 16 of the stomach 3, which are to be secured together, the endoscope 24 may be withdrawn from the stomach 3, and the multiband ligator 80 is removed from the distal end 22 thereof, and the endoscope 24 is again urged orally through the oesophagus into the stomach 3 of the subject. The suturing instrument 8 described with reference to FIG. 9 is then urged through the instrument channel 26 in the endoscope 24 into the stomach 3.

With the distal end 63 of the outer sleeve 60 of the suturing instrument 8 located in the stomach 3, the suturing instrument 8 is operated in a similar manner as already described with reference to FIGS. 9 to 24 but without the invaginating device 10, for sequentially inserting the sutures 50 in the respective pairs of the corresponding invaginated parts 42. In general, it is envisaged that the sutures 50 will be inserted sequentially into the pairs of the invaginated parts 42 from the distal most pair of the invaginated parts 42 to the proximal most pair thereof.

When the sutures 50 have been inserted in all of the pairs of the invaginated parts 42, the suturing instrument 8 is operated as already described for sequentially tightening the sutures 50 by urging the cinch clip 57 of each suture 50 along the loop 55 thereof towards the anchor elements 54, for in turn reducing the effective length of the suture thread 51 of each suture 50 between the anchor elements 54 to urge the corresponding invaginated parts 42 of each pair thereof tightly together. The sutures 50 are typically sequentially tightened commencing with the distal most suture 50a and finishing with the proximal most suture 50e to form the sleeve 6 in the stomach 3.

Alternatively, in this embodiment of the invention after each suture 50 has been inserted in the corresponding pair of the invaginated parts 42, the suture 50 of that pair of invaginated parts 42 may be tightened before the next suture 50 is inserted in the next pair of the invaginated parts 42.

In some cases it may not be necessary to remove the multiband ligator from the endoscope where the presence of the multiband ligator does not impede passing the suturing instrument 8 through the endoscope into the stomach 3. In such cases on completion of the formation of the last of the invaginated parts by the multiband ligator 80, the endoscope 24 with the multiband ligator 80 remains in the stomach 3, and the suturing instrument 8 described with reference to FIG. 9 is urged through the instrument channel 26 of the endoscope 24 into the stomach 3.

It is envisaged that in some embodiments of the invention described with reference to FIGS. 1 to 24 the suturing instrument 8 may be provided with a pusher element 69 without the hook 70, and thus, the suturing instrument 8 would not be suitable for tightening the sutures 50, whereby the cinch clip 57 is urged along the loop 55 of the suture 50 towards the anchor elements 54 thereof. In which case, the suturing instrument 8 would be used solely for the purpose of inserting the sutures 50 into the respective pairs of the invaginated parts 42. The tightening of the sutures 50 would be carried out by a separate suture tightening instrument.

Such a suture tightening instrument could comprise a flexible outer sleeve, similar to the outer sleeve 60 of the suturing instrument 8, with the outer sleeve having an elongated longitudinally extending bore similar to the bore 62 extending therethrough. The outer sleeve would terminate in a distal end defining a distal abutment face, similar to the distal abutment face 64 defined by the distal end of the outer sleeve 60 of the suturing instrument 8. An elongated flexible operating rod, which could be similar to the pusher element 69 of the suturing instrument 8 would be located in the bore of the outer sleeve, and would be slideable therein. The operating rod would terminate at a distal end in a hook similar to the hook 70 of the pusher element 69. The operating rod would be connected to a proximal operating element located in a handle of the suture tightening instrument, and the operating element would be configured for urging the operating rod proximally and distally in the bore of the outer sleeve. Such a suture tightening instrument is disclosed in the applicant's PCT specification No. WO 2021/165945, the disclosure of which is incorporated herein by reference.

The operating rod would be urged distally in the bore of the outer sleeve to urge the hook at the distal end thereof into a distal state extending outwardly from the outer sleeve for engaging a loop 55 of one of the sutures 50 to be tightened. The operating element would be adapted to urge the operating rod proximally through the bore of the outer sleeve, for in turn urging the hook with the loop of the suture engaged thereon from the distal state into the bore of the outer sleeve until the cinch clip abutted the distal abutment face at the distal end of the outer sleeve. Further urging of the operating rod and in turn the hook proximally in the bore of the outer sleeve would urge the cinch clip 57 along the loop 55 of the suture 50 towards the anchor elements 54, for in turn reducing the effective length of the suture between the anchor elements 54, for in turn tightening the suture to draw and secure the invaginated parts of the corresponding pair thereof tightly together.

In cases where the suturing instrument 8 is provided without the hook 70 on the distal end of the pusher element 69, on completion of insertion of the sutures 50 into the pairs of the invaginated parts 42, the suturing instrument 8 would be withdrawn through the instrument channel 26 of the endoscope 24, and the suture tightening instrument would be inserted through the instrument channel 26 of the endoscope 24 into the stomach 3 for carrying out the sequential tightening of the sutures 50 in the stomach.

Needless to say, it will be readily apparent to those skilled in the art that a suturing instrument without a hook 70 located on the distal end of the pusher element thereof, may be used in methods for endoscopically suturing two parts of the interior of any lumen, vessel or organ together interiorly in the lumen, vessel or organ. In which case, the suturing instrument would be used to insert a suture into a pair of the invaginated parts of the lumen, vessel or organ, or to inset sutures into respective pairs of invaginated parts in the lumen, vessel or organ and the suture tightening instrument would then be used to sequentially tighten the suture or sutures already inserted in the pair or respective pairs of invaginated parts.

Referring now to FIG. 30 there is illustrated a portion of a wall 85 of a lumen, vessel or organ of a subject. The lumen, vessel or organ may be any lumen, vessel or organ within a subject. In this embodiment of the invention a region 86 of the wall 85 of the lumen, vessel or organ has been weakened by scraping an inner surface 87 thereof in the region 86 to remove, for example, a cancerous growth, thereby leading to thinning of the wall 85 in the region 86 which would ultimately lead to weakening of the region 86. In this embodiment of the invention it is desired to suture opposite edges 88 of the weakened region 86 together in case the weakened region 86 should subsequently perforate.

The edges 88 of the weakened region 86 may be sutured together using the method described with reference to FIGS. 1 to 24 which comprises using the suturing apparatus 1 comprising the suturing instrument 8 and the invaginating device 10, or the method described with reference to FIGS. 25 to 29 using the suturing instrument 8 and the multiband ligator 80. However, in this case, the edges 88 of the weakened region 86 are sutured together by the method according to the invention using the suturing instrument 8 and the multiband ligator 80.

The multiband ligator 80 as described with reference to FIGS. 25 to 29 is attached to the end of an endoscope which is appropriately entered through an orifice of the body of the subject into the lumen, vessel or organ. The appropriate number of invaginated parts 42 are formed adjacent the opposite edges 88 of the weakened region 86 by the multiband ligator 80, and are retained invaginated by bands 90 as described with reference to the method of FIGS. 25 to 29.

With the invaginated parts 42 formed and banded adjacent the opposite edges 88 of the weakened region 86 by the multiband ligator 80, if necessary the endoscope is withdrawn from the lumen, vessel or organ, and the multiband ligator 80 is removed from the endoscope, and the endoscope is reinserted in the lumen, vessel or organ. Otherwise, the endoscope with the multiband ligator 80 thereon remains in the stomach.

The suturing instrument 8 is then entered into the lumen, vessel or organ through the instrument channel of the endoscope. The sutures 50 are then inserted sequentially in the pairs of the corresponding invaginated parts 42 on the respective opposite edges 88 of the weakened region 86. On completion of insertion of the sutures the sutures are then tightened for securing the pairs of invaginated parts 42 together, as already described with reference to the method of FIGS. 24 to 29.

However, it will be readily apparent to those skilled in the art that the opposite edges 88 of the weakened region 86 of the wall 85 of the lumen, vessel or organ could also be secured together by the method described with reference to FIGS. 1 to 24 using the suturing apparatus 1 comprising the suturing instrument 8 and the invaginating device 10.

It will also be appreciated that both methods according to the invention could also be used in suturing an incision in a wall of a lumen, vessel or organ internally in the lumen, vessel or organ, whereby invaginated parts formed on the wall of the lumen, vessel or organ on the inner side thereof along opposite sides of the incision, would be formed in pairs, and sutured together as already described to close the incision.

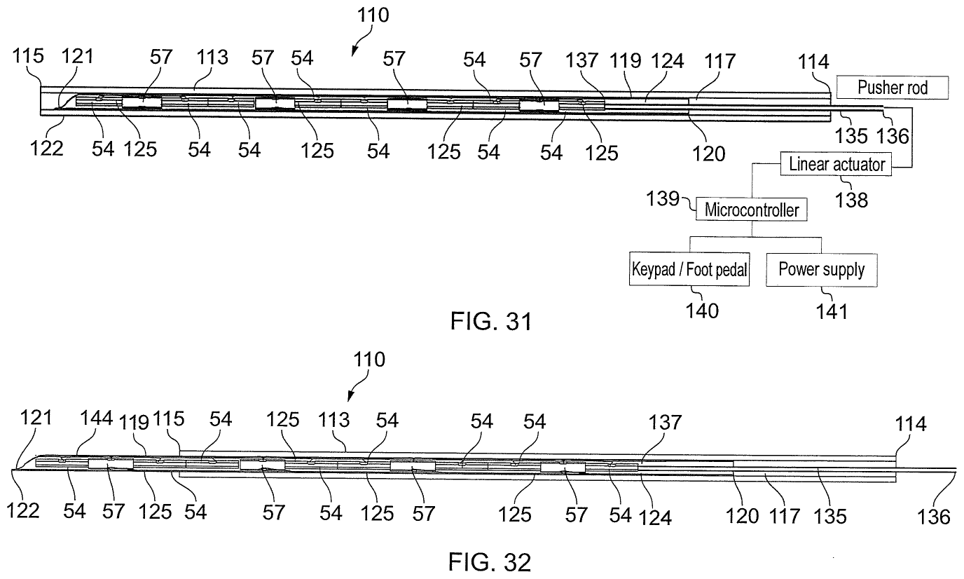

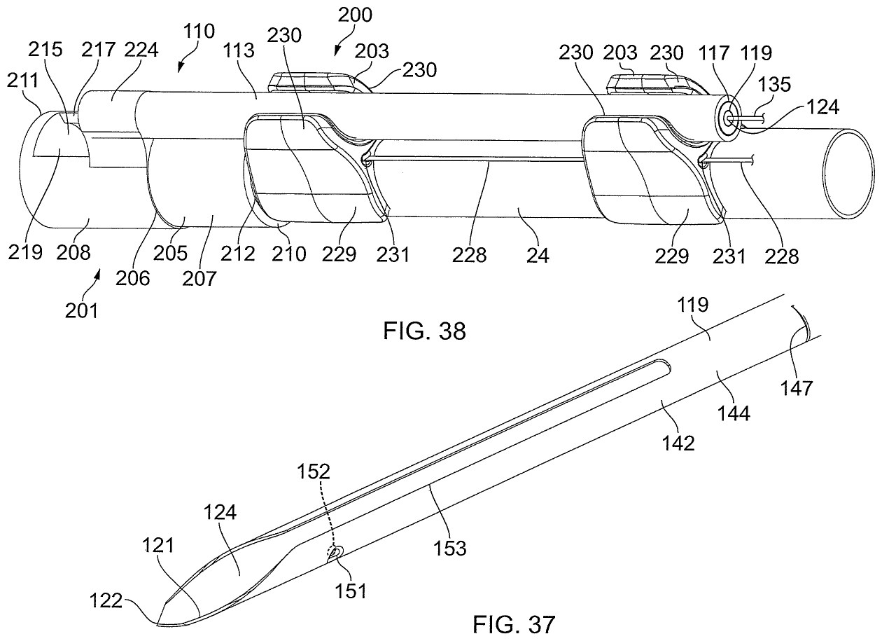

Referring now to FIGS. 31 to 37, there is illustrated a suturing instrument also according to the invention indicated generally by the reference numeral 110. The suturing instrument 110 is suitable for use in the method described with reference to FIGS. 1 to 24 for suturing two portions of a lumen, vessel or organ together with the suturing instrument 110 located internally within the interior of the lumen, vessel or organ, such as in the forming of a gastric sleeve 6 in an endoscopic sleeve gastroplasty procedure using the invaginating device 10. When the suturing instrument 110 is used in the suturing method described with reference to FIGS. 1 to 24, the suturing instrument 110 would be used in conjunction with the invaginating device 10. The suturing instrument 110 is also suitable for use in the method described with reference to FIGS. 25 to 29 for suturing two portions of a lumen, vessel or organ together with the suturing instrument 110 located internally within the interior of the lumen, vessel or organ, such as in the forming of a gastric sleeve 6 in an endoscopic sleeve gastroplasty procedure whereby all the parts 42 at the locations 17 of the front and rear walls 15 and 16 of the stomach 3 are invaginated before any suturing of the invaginated parts 42 commences, for example, where the invaginated parts are invaginated by the multiband ligator 80. The suturing instrument 110 is also suitable for use in the method described with reference to FIG. 30 where two portions of a lumen, vessel or organ are sutured together with the suturing instrument located within the lumen, vessel or organ.

The suturing instrument 110 comprises an elongated outer sleeve 113 of circular transverse cross-section, extending between a proximal end 114 and a distal end 115. An elongated cannula accommodating bore 117 of circular transverse cross-section extends longitudinally through the outer sleeve 113 from the proximal end 114 to the distal end 115 for accommodating a cannula 119 extending longitudinally therein. The cannula 119 extends from a proximal end 120 to a distal end 121, and terminates in its distal end 121 in a distal piercing tip 122. The cannula 119 is of circular transverse cross-section and is slideable in the cannula bore 117 of the outer sleeve 113. An elongated suture accommodating bore 124 of circular transverse cross-section extends longitudinally through the cannula 119 from the proximal end 120 to the distal end 121 for accommodating a plurality of sutures 125 therein.

The sutures 125 are substantially similar to the sutures 50 illustrated in FIG. 8, and similar components are identified by the same reference numerals. In this embodiment of the invention the anchor elements 54 of the sutures 125 are of a circular cross-section and of a diameter to be freely slideable in the suture accommodating bore 124. The maximum transverse cross-sectional dimension of the cinch clips 57 of the sutures 125 is such that the cinch clips 57 of the sutures 125 are freely slideable in the suture accommodating bore 124 of the cannula 119. Additionally, in this embodiment of the invention the length of the anchor accommodating elements 54 of each suture 125 is the same, and the length of the anchor accommodating elements 54 of the respective sutures 125 are also the same. The length of the cinch clip 57 of each suture 125 is the same as the length of each of the anchor elements 54 of the suture 125. Additionally, the lengths of the cinch clips 57 of the sutures 125 are the same for all the sutures 125. The reason for these dimensions of the anchor elements 54 and the cinch clips 57 of the sutures 125 will be described in more detail below.

The proximal end 114 of the outer sleeve 113 terminates in a handle (not shown). The cannula 119 is slideable in the outer sleeve 113 from a withdrawn state illustrated in FIG. 31 to protect the distal piercing tip 122, to an extended state illustrated in FIG. 32 for piercing tissue into which the sutures 125 are to be inserted during suturing of the lumen, vessel or organ. An operating lever (not shown) located in the handle (not shown) is connected to the cannula 119 for operating the cannula 119 in the cannula bore 117 of the outer sleeve 113 between the withdrawn state and the extended state.

A pusher element, in this embodiment of the invention a pusher rod 135 extending from a proximal end 136 to a distal end 137 is of circular external transverse cross-section. The external diameter of the pusher rod 135 is less than the diameter of the suture accommodating bore 124 of the cannula 119, so that the pusher rod 135 is slideable in the suture accommodating bore 124. The pusher rod 135 is urgeable in the suture accommodating bore 124 in a distal direction, for selectively and sequentially discharging the anchor elements 54 and the cinch clips 57 of the sutures 125 from the suture accommodating bore 124 through the distal piercing tip 122 of the cannula 119, during suturing of the portions of the lumen, vessel or organ as will be described below. The distal end 137 of the pusher rod 135 is configured to engage the proximal most anchor element 54 of the proximal most suture 125 in the suture accommodating bore 124 of the cannula 119, so that as the pusher rod 135 is selectively and incrementally urged distally through the suture accommodating bore 124, the anchor elements 54 and the cinch clips 57 are sequentially discharged from the suture accommodating bore 124 through the distal piercing tip 122 of the cannula 119.

An urging means, in this embodiment of the invention comprising an actuator 138, which in this case comprises a linear actuator illustrated in block representation in FIG. 31, is configured to incrementally urge the pusher rod 135 through the suture accommodating bore 124 for selectively and sequentially discharging the anchor elements 54 and the cinch clips 57 from the suture accommodating bore 124 through the distal piercing tip 122 of the cannula 119. The actuator 138 may comprise an electrically powered linear motor, or an electrically powered stepper motor, which would transmit drive to the pusher rod 135, through for example, a rack and pinion drive.

A controller, in this embodiment of the invention a signal processor provided by a programmable microcontroller 139 is programmed to be responsive to an activation signal to operate the linear actuator 138 to urge the pusher rod 135 distally in the suture accommodating bore 124 a predefined distance. The length of the predefined distance through which the pusher rod 135 is urged in response to the activation signal is equal to the length of one of the anchor elements 54. Since the length of the anchor elements 54 and the length of the cinch clips 57 are the same, each time the pusher rod 135 is urged distally in the suture accommodating bore 124 through one of the predefined distances, the distal most one of the anchor elements 54 or the cinch clip 57 of the distal most suture 125 in the suture accommodating bore 124 adjacent the distal piercing tip 122 of the cannula 119 is urged from the suture accommodating bore 124 through the distal piercing tip 122.

The activation signal may be generated or derived from any suitable source. In this embodiment of the invention the activation signal is produced by a human operable switch, which in this case comprises a foot operated switch (not shown), operated by a foot pedal 140. The switch is configured, so that each time it is operated by the foot pedal 140 it produces an activation signal. The microcontroller 139 is responsive to each activation signal from the foot pedal 140 for operating the linear actuator 138 for in turn urging the pusher rod 135 through one predefined distance. Although in some embodiments of the invention instead of or as well as a foot switch, a key pad may be provided for producing the activation signal.

A power supply 141 is provided for supplying power to the microcontroller 139 and in turn to the linear actuator 138 for powering thereof and to the switch of the foot pedal 140. The linear actuator 138 and the microcontroller 139 are located in the handle (not shown) for operating the pusher rod 135.

In this embodiment of the invention the sutures 125 are located in the suture accommodating bore 124 of the suturing instrument 110 with the cinch clip 57 of each suture 125 located between the anchor elements 54 thereof. Thus, when the pusher rod 135 is urged distally through the first one of the predefined distances in the suture accommodating bore 124, the distal most anchor element 54 of the distal most suture 125 is initially urged through the distal piercing tip 122 of the cannula 119. On the pusher rod 135 being urged distally in the suture accommodating bore 124 through the next predefined distance, the cinch clip 57 of the distal most suture 125 in the suture accommodating bore 124 of the cannula 119 is next urged through the distal piercing tip 122 of the cannula 119. On the next urging of the pusher rod 135 distally through the next one of the predefined distances, the second one of the anchor elements 54, which is now the distal most one of the anchor elements 54 of the distal most suture 125 in the suture accommodating bore 124 is urged through the distal piercing tip 122 of the cannula 119, and so on as the sutures are being sequentially discharged from the suture accommodating bore 124 of the suturing instrument 110 through the distal piercing tip 122 by the pusher rod 135. However, it will be appreciated that in some embodiments of the invention the sequence in which the anchor elements and the cinch clips of the sutures 125 are located in the suture accommodating bore 124 of the cannula 119 of the suturing instrument 110 may be different, for example, in some embodiments of the invention the anchor elements 54 of each suture 125 may be located together, and the cinch clip 57 may be located either distally or proximally of the anchor elements of the corresponding suture 125.

The outer sleeve 113 of the suturing instrument 110 is of a flexible bendable material in order to accommodate bending and flexing of the outer sleeve 113 during maneuvering of the suturing instrument 110 through various bends and curves in the anatomy of a subject through which the suturing instrument 110 must be passed in order to access the lumen, vessel, organ or cavity in which the suturing is to be carried out. In this embodiment of the invention the material of the outer sleeve 113 comprises a PTFE material, and is formed by extruding the PTFE material. However, it will be understood that the outer sleeve 113 may be of any other suitable material, and in some embodiments of the invention the outer sleeve 113 may comprise a braided cable, for example, a Bowden cable.

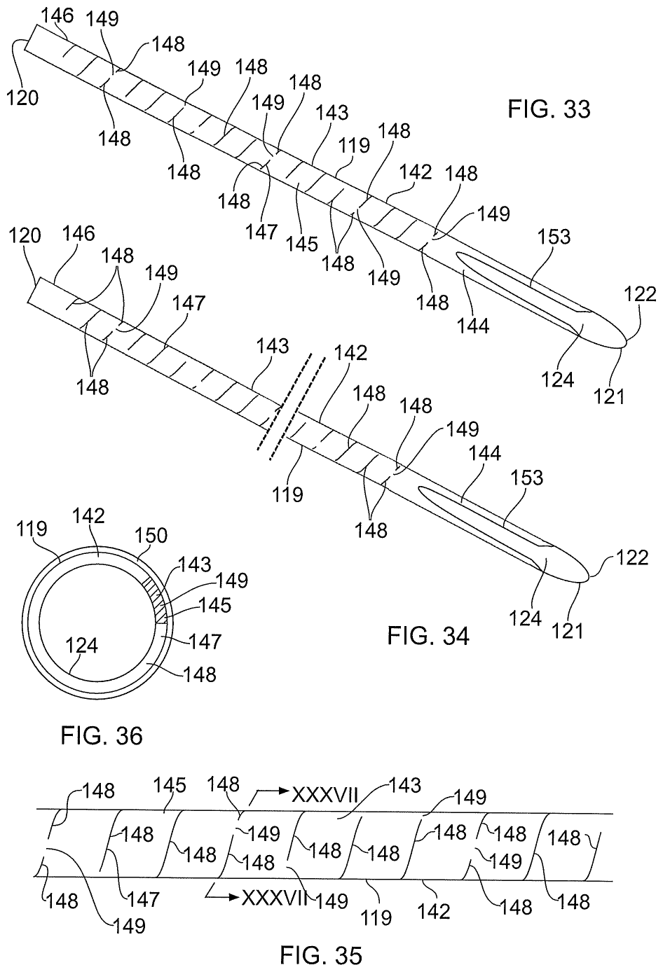

Referring now to FIGS. 33 to 37, the cannula 119 is also of a flexible bendable material in order to accommodate maneuvering of the suturing instrument 110 through the various bends and curves through which the suturing instrument must be passed to gain access to the lumen, vessel or organ. In this embodiment of the invention the cannula 119 comprises a tubular member 142 of stainless steel material of constant circular transverse cross-section and having a peripheral wall 143 of constant radial thickness along its length. The outer diameter of the tubular member 142 is approximately 1.27 mm, and the radial thickness of the wall 143 of the tubular member 142 is approximately 0.0762 mm. The tubular member 142 terminates in a distal piercing portion 144 which is substantially rigid for piercing through tissue of the wall of the lumen, vessel or organ which is to be sutured. An intermediate portion 145 extends from the distal piercing portion 144 to a proximal end portion 146 of the tubular member 142.

An interrupted helical slot 147 extends through the peripheral wall 143 of the tubular member 142 and extends along the intermediate portion 145 in order to provide the intermediate portion 145 with flexibility and bendability, so that the intermediate portion 145 is sufficiently bendable and flexible for maneuvering the suturing instrument through the various bends and curves through which the suturing instrument 110 must be passed in order to gain access to the lumen, vessel or organ in which suturing is to be carried out. In this embodiment of the invention the interrupted helical slot 147 comprises a plurality of helical slot sections 148 interrupted by a plurality of interrupted sections 149. Each interrupted section 149 is located between an adjacent pair of the slot sections 148. Each slot section 148 extends partly around the tubular member 142 an angular distance of approximately 240°, and each interrupted section 149 of the interrupted helical slot extends partly around the tubular member 142 an angular distance of approximately 40°. The pitch of the interrupted helical slot 147 is approximately 1.2 mm, and the kerf width of the slot of each slot section 148 is approximately 0.025 mm.

The tubular member 142 is sheathed with a fine sleeve 150 of extruded polymer material heat shrunk onto the tubular member 142 over the entire length of the tubular member 142, see FIG. 36, extending across the helical slots 147. The polymer may be any suitable heat shrinkable polymer material, for example, FEP or PTFE.

A retaining means for releasably retaining the distal most one of the anchor elements 54 or the cinch clip 57, as the case may be, of the distal most one of the sutures 125 in the suture accommodating bore 124 comprises an inwardly impressed dimple 151 formed on one side of the peripheral wall 143 in the distal piercing portion 144 of the cannula 119 adjacent the distal piercing tip 122 to form a corresponding inwardly extending retaining projection 152 extending inwardly into the suture accommodating bore 124. A relieving slot 153 extends longitudinally from the distal piercing tip 122 into the distal piercing portion 144 of the cannula 119 in order to permit outward movement of the dimpled portion of the tubular member 142 adjacent the dimple 151 in order to allow outward movement of the retaining projection 152 for releasing the distal most one of the anchor elements 54 or the cinch clip 57, as the case may be, of the distal most one of the sutures 125 as the pusher rod 135 is being urged distally in the suture accommodating bore 124 through the predefined distance for discharging the distal most one of the anchor elements 54 or the cinch clip 57 from the suture accommodating bore 124 through the distal piercing tip 122. The inherent resilience of the tubular member 142 is sufficient to return the portion of the tubular member 142 with the dimple 151 to its normal undisturbed state when the distal most one of the anchor elements 54 or the cinch clip 57 has passed the retaining projection 152, so that the next distal most one of the anchor elements 54 or the cinch clip 57 is engaged by the retaining projection 152 as it returns to its undisturbed state, and is retained captive by the retaining projection 152, until the pusher rod 135 is urged further along the suture accommodating bore 124 through the next one of the predefined distances for urging that next one of the anchor elements 54 or the cinch clip 57 passed the retaining projection 152 from the suture accommodating bore 124.

While the cannula 119 has been described as being of tubular steel, the cannula 119 may be of any other suitable material, and in some embodiments of the invention may be of a plastics or polymer material which would be inherently flexible and bendable, but over a short length, for example, over the length of the distal piercing portion 144 would be substantially rigid, and would be of sufficient rigidity to achieve piercing of the tissue to be pierced by the distal piercing portion 144 of the cannula 119. Needless to say, while specific linear and angular dimensions of the interrupted helical slot, and the slot sections and the interrupted sections of the interrupted helical slot have been described, it will be readily understood that the interrupted helical slot may be of any other suitable angular and linear dimensions.

Needless to say, in cases where the tubular member of the cannula is of an inherently flexible bendable material, the intermediate portion of the tubular member may be provided without an interrupted helical slot.

The advantage of providing an interrupted helical slot in the intermediate portion 145 of the tubular member which is insufficiently flexible and bendable for passing through the various bends and curves in a subject through which the suturing instrument must pass in order to gain access to the lumen, vessel or organ is that the interrupted helical slot while providing the necessary degree of flexibility and bendability does not affect the linear dimensional stability of the tubular member, so that the length of the intermediate portion 145 of the tubular member 142 remains constant during bending and flexing thereof, and during urging of the cannula to pierce through a part of the lumen, vessel or organ during suturing thereof.

The pusher rod 135 is likewise of a flexible bendable material, in this embodiment of the invention a stainless steel material. However, it is envisaged that the pusher rod may comprise an elongated wire of nitinol titanium, and such a pusher rod would have a considerable degree of flexibility and bendability.

Use of the suturing instrument 110 in suturing two portions of a lumen, vessel or organ internally in the lumen, vessel or organ, for example, for use in the formation of a gastric sleeve in an endoscopic sleeve gastroplasty procedure by the method described with reference to FIGS. 25 to 29, is substantially similar to the use of the suturing instrument 8 in the method of FIGS. 25 to 29. However, in the case of the suturing instrument 110, since the suture accommodating bore 124 of the cannula can be charged with a plurality of the sutures 125, it is not necessary to withdraw the suturing instrument 110 from the stomach or other lumen, vessel or organ after each suture has been inserted in the corresponding pair of invaginated parts for recharging with the next suture. In general, the suture accommodating bore 124 of the suturing instrument 110 is charged with the appropriate number of sutures 125, prior to entering of the suturing instrument 110 into the stomach 3 or other lumen, vessel or organ.

In the suturing of the front and rear walls 15 and 16 of the stomach 3 in an endoscopic sleeve gastroplasty procedure being carried out in accordance with the method described with reference to FIGS. 25 to 29, using the suturing instrument 110, the endoscope 24 is entered orally through the oesophagus into the stomach. Initially, the invaginated parts 42 are formed in pairs on the respective front and rear walls 15 and 16 of the stomach 3 by any suitable invaginating device, for example, a multiband ligator similar to the multiband ligator 80 described with reference to the method of FIGS. 25 to 29. The multiband ligator may be entered through the instrument channel of the endoscope 24, or may be attached to the distal end of the endoscope prior to entry of the endoscope 24 into the stomach 3. On completion of the formation of the pairs of the invaginated parts 42, if the invaginating device such as the multiband ligator 80 had been entered into the stomach through the instrument channel of the endoscope, the multiband ligator would be withdrawn from the stomach through the endoscope 24. The suturing instrument 110 with the cannula 119 in the withdrawn state within the outer sleeve 113 as illustrated in FIG. 31 and charged with the sutures 125, is then entered into the stomach 3 through the instrument channel of the endoscope 24. If on the other hand the multiband ligator had been attached to the distal end of the endoscope, in general, the endoscope would be withdrawn from the stomach for detaching the multiband ligator 80 from the distal end thereof. The endoscope would then be re-entered into the stomach orally through the oesophagus. With the endoscope in the oesophagus, the suturing instrument 110 is entered through the instrument channel of the endoscope into the stomach, and the first of the sutures 125 is inserted in the first of the pairs of the invaginated parts 42 on the respective front and rear walls 15 and 16 of the stomach.

To insert the first of the sutures 125 into the first pair of the invaginated parts 42, the cannula 119 is urged distally in the cannula bore 117 of the outer sleeve 113 by the operating lever (not shown) in the handle (not shown) of the suturing instrument 110 to in turn urge the distal piercing tip 122 and the distal piercing portion 144 of the cannula 119 outwardly from the distal end 115 of the outer sleeve 113. The distal piercing tip 122 is pierced through the first one of the invaginated parts of the first pair thereof so that the distal piercing tip 122 exits the first of the invaginated parts 42 on the distal side 79 thereof. With the distal piercing tip extending through the invaginated part 42, the foot pedal 140 of the foot pedal switch is operated to produce one of the activation signals. The microcontroller 139 in response to the activation signal operates the linear actuator 138 to urge the pusher rod 135 through one of the predefined distances for discharging the first of the anchor elements 54 from the suture accommodating bore 124, which is the distal most one of the anchor elements 54 of the distal most suture 125 in the suture accommodating bore 124 of the cannula 119 on the distal side 79 of the first of the invaginated parts 42. The cannula 119 is then withdrawn from the first one of the invaginated parts 42 with the first anchor element 54 on the distal side of the first of the invaginated part 42, and with the suture thread 51 extending from the first of the anchor elements 54 through the invaginated part 42.

The foot pedal 140 is activated again to produce another activation signal. The microcontroller 139 in response to the activation signal operates the linear actuator 138 to urge the pusher rod 135 further distally through another one of the predefined distances for in turn urging the cinch clip 57 of the distal most suture 125 in the suture accommodating bore 124 of the cannula 119 through the distal piercing tip 122 and into the stomach 3.

The distal piercing tip 122 of the cannula 119 is then urged through the second one of the pair of the invaginated parts 42, and the foot pedal switch 140 is again operated to produce another activation signal. The microcontroller 139 in response to the activation signal operates the linear actuator 138 to urge the pusher rod 135 distally through a further one of the predefined distances to discharge the second one of the anchor elements 54, which is now the distal most anchor element in the suture accommodating bore 124 of the distal most suture 125 therein on the distal side 79 of the second one of the invaginated parts 42. The cannula 119 is withdrawn from the second one of the invaginated parts 42 with the suture thread 51 extending from the second one of the anchor elements 54 of the suture 125 through the second invaginated part 42 of the pair of the invaginated parts, and with the loop 55 of the suture thread 51 extending through the cinch clip 57.

The cannula is then moved to the first one of the next pair of the invaginated parts 42 to insert the next, which is now the distal most suture 125 in the suture accommodating bore 124 into that invaginated part as already described, and so on until the sutures 125 have been inserted in all the pairs of the invaginated parts 42. Thereafter, the suturing instrument 110 is withdrawn from the stomach through the instrument channel of the endoscope 24, and a tightening instrument is then inserted through the instrument channel of the endoscope 24 into the stomach for sequentially tightening the sutures as already described with reference to the method of FIGS. 25 to 29.

The use of the suturing instrument 110 when suturing two portions of a lumen, vessel or organ internally in the lumen, vessel or organ, for example, for use in the formation of a gastric sleeve in an endoscopic sleeve gastroplasty procedure in conjunction with the invaginating device 10 based on the method described with reference to FIGS. 1 to 24 is substantially similar to the use of the suturing instrument 8 described with reference to FIGS. 1 to 24, with the exception that as each suture 125 is inserted in the invaginated parts of the corresponding pair thereof, there is no need to withdraw the suturing instrument 110 from the stomach, or other lumen, vessel or organ for recharging of the suturing instrument with the next suture. In this case, the sutures 125 are sequentially discharged from the suture accommodating bore 124 of the cannula 119 as the invaginated parts of the pairs thereof are being formed. Otherwise, use of the suturing instrument 110 in conjunction with the invaginating device 10 is similar to the use of the suturing instrument 8 with the invaginating devices 10.

Tightening of the sutures 125 when the sutures have been inserted in the invaginated parts of all the pairs thereof, may be carried out using a separate tightening instrument, or alternatively, the pusher rod 135 adjacent the distal end 137 thereof may be provided with a hook, similar to the hook 70 located adjacent the distal end of the pusher element 69. In which case, the microcontroller 139 would be responsive to a tightening signal which may be inputted to the microcontroller 139 through any suitable interface device for firstly, urging the pusher rod 135 distally in the cannula 117 for in turn urging the hook adjacent the distal end 137 of the pusher rod 135 outwardly of the distal end 121 of the cannula 119. The hook would then be engaged with the loop 55 of a corresponding one of the sutures 50. The microcontroller 139 would then be configured to be responsive to a second tightening signal entered into the microcontroller 137 through a suitable interface to operate the linear actuator 138 for urging the pusher rod 135 proximally in the cannula 119 for in turn drawing the loop 55 of the suture 50 into the suture accommodating bore 124 of the cannula 119 and in turn into the cannula accommodating bore 117 of the distal end 115. On the cinch clip 57 of the suture 50 engaging the distal end 115 of the outer sleeve 113 further drawing of the loop into the suture accommodating bore 124 of the cannula 119 tightens the suture 50 for drawing the invaginated parts of the corresponding pair thereof together.

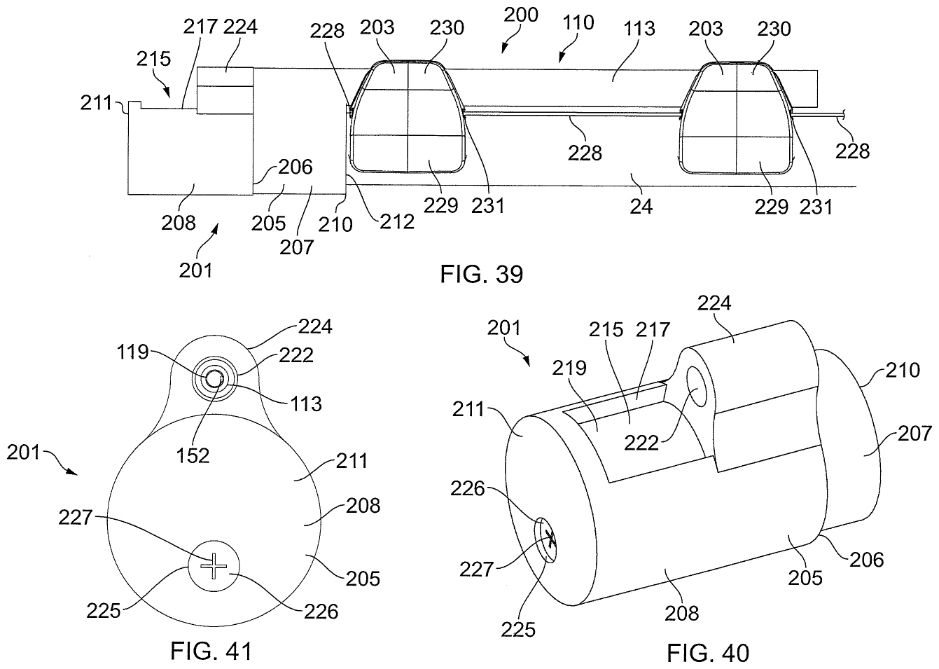

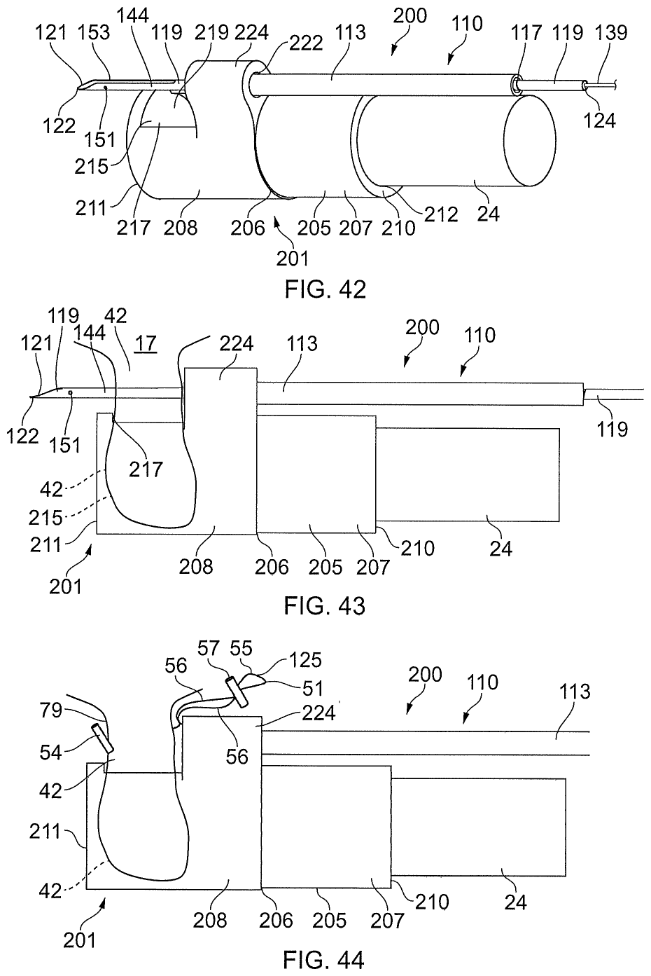

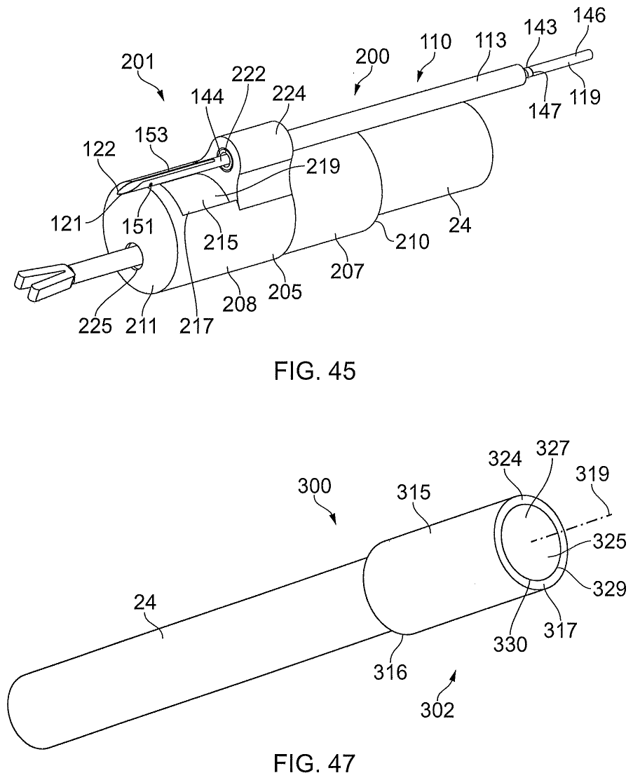

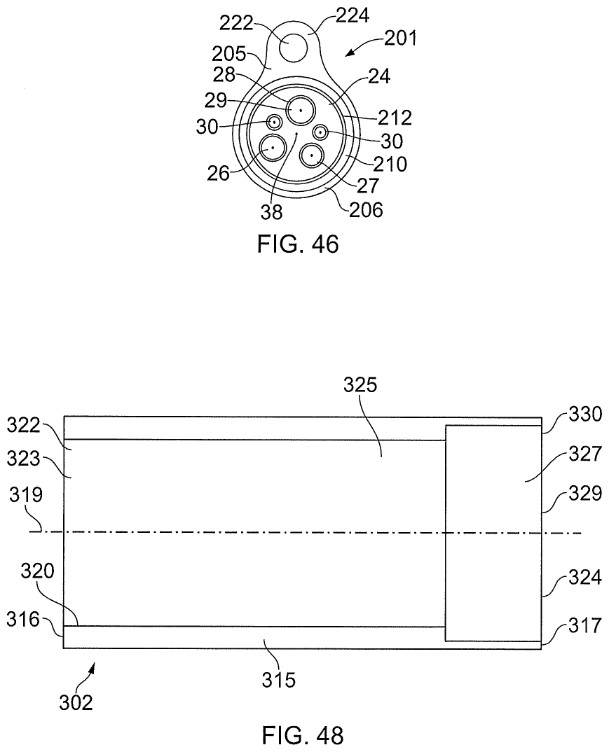

Referring now to FIGS. 38 to 46, there is illustrated a suturing apparatus 200 according to another embodiment of the invention for suturing two portions of a lumen, vessel or organ internally in the lumen, vessel or organ for example, in the stomach during an endoscopic sleeve gastroplasty procedure, in which a gastric sleeve, similar to the gastric sleeve 6 described with reference to the methods of FIGS. 1 to 24 is formed.

The suturing apparatus 200 comprises an invaginating device also according to the invention and indicated generally by the reference numeral 201, and the suturing instrument 110, a portion only of which is illustrated in FIGS. 38 to 44. The invaginating device is adapted for coupling to a dual channel endoscope 24, which is similar to the dual channel endoscope 24 described with reference to the invaginating device 10 of FIGS. 1 to 7, and similar components are identified by the same reference numerals. However, instead of the suturing instrument 110 being entered into the lumen, vessel or organ, in which the suturing is to be carried out through the instrument channel 26 of the endoscope 24, in this embodiment of the invention, the suturing instrument 110 is releasably secured to the endoscope 24 and extends longitudinally along the endoscope 24 along an outer side of the endoscope 24. The suturing instrument 110 is releasably secured to the endoscope by a plurality of clips 203, as will be described below which are releasably attached to both the endoscope 24 and the suturing instrument 110 at spaced apart intervals longitudinally along the endoscope 24 and the suturing instrument 110. Before describing the securing of the suturing instrument 110 to the endoscope 24, the invaginating device 201 will first be described.

The invaginating device 201 is somewhat similar to the invaginating device 10 described with reference to FIGS. 1 to 7, and comprises a body member 205 of circular transverse cross-section stepped at 206 to form a proximal cylindrical portion 207 and a distal cylindrical portion 208 of diameter slightly greater than the diameter of the proximal cylindrical portion 207. The body member 205 extends from a proximal end 210 adjacent the proximal end of the proximal cylindrical portion 207 to a distal end 211 adjacent the distal end of the distal cylindrical portion 208. A connector for connecting the body member 205 to the distal end 22 of the endoscope 24 is provided by a socket 212 extending axially into the proximal cylindrical portion 207 from the proximal end 210 thereof for releasably engaging and securing the body member 205 to the distal portion 25 adjacent a distal end 22 of the endoscope 24.

A suction chamber 215 extends radially into the distal cylindrical portion 208 from an access opening 217. In this embodiment of the invention the suction chamber 215 is formed by a suction cavity 219 of substantially cylindrical shape. A suction bore (not shown) but similar to the suction bore 40 of the invaginating device 10 extends proximally from the suction chamber 215 to the socket 212. The suction bore (not shown) communicates the suction chamber 215 with the instrument channel 26 of the endoscope 24, to which suction is applied for invaginating parts of the lumen, vessel or organ into the suction chamber 215, as for example, the invaginated parts 42 of the front and rear walls 15 and 16 of the stomach 3, as illustrated in FIGS. 10 to 24.

An instrument passageway, in this embodiment of the invention provided by a guide bore 222 extends through a guide flange 224 extending radially outwardly from the distal cylindrical portion 208 of the body member 205 adjacent the suction chamber 215 and between the step 206 and the suction chamber 215. The guide bore 222 is configured for guiding the distal piercing tip 122 of the cannula 119 of the suturing instrument 110 into an invaginated part 42 which is retained invaginated in the suction chamber 215. The guide flange 224 extends radially from the body member 205 in a direction opposite to the radial direction into which the suction chamber 215 extends into the body member 205. Further, the guide flange 224 extends radially from the body member 205 in the same direction as that in which the access opening 217 to the suction chamber 215 faces.

An instrument bore 225 extends through the body member 205 from the socket 212 to the distal end 211 of the body member 205 and through the distal end 211 for accommodating instruments from the instrument channel 27 of the endoscope 24 through the body member 205 of the invaginating device 201. A sealing membrane 226 seals the distal end of the instrument bore 225 to minimise air being drawn through the instrument channel 27 of the endoscope 24 while suction is applied to the suction chamber 215. A cross-slit 227 is formed in the membrane 226 to accommodate an instrument therethrough.

The body member 205 comprises a polymer material, and the guide flange 224 is formed integrally with the body member 205 by injection moulding. The polymer material of the body member 205 in this embodiment of the invention comprises a transparent material in order to accommodate viewing of the interior of the lumen, vessel or organ in which the invaginating device 201 is located through the vision channel 28 of the endoscope 24. However, it will be readily apparent to those skilled in the art that the body member 205 need not be of a transparent material, and in cases where the body member is of a translucent or opaque material, a viewing bore, a viewing aperture or a viewing channel would be provided in or extending through the body member 205 and would be configured to communicate with the vision channel 28 of the endoscope 24 to facilitate viewing of the interior of the lumen, vessel or organ.

Turning now to the clips 203, each clip 203 is of a resilient material, and comprises two pairs of jaws, namely, a pair of jaws 229 which resiliently and releasably engage the endoscope 24, and a pair of jaws 230 which resiliently and releasably engage the outer sleeve 113 of the suturing instrument 110. A retaining means, in this embodiment of the invention an endless ligature 228 extends through pairs of spaced apart ligature accommodating bores 231, which extend through the clips 203 for retaining the clips 203 connected together in case one of the clips 203 became detached from the endoscope 24 and the suturing instrument 110. The suturing instrument 110 is secured to the endoscope 24 with the outer sleeve 113 of the suturing instrument 110 engaged in the guide bore 222 of the guide flange 224.

The use of the suturing apparatus 200 is substantially similar to the use of the suturing apparatus 1 described with reference to FIGS. 9 to 24 in suturing two portions of a lumen, vessel or organ within the lumen, vessel or organ, for example, for suturing the front and rear walls 15 and 16 of the stomach 3 to form a gastric sleeve 6 to reduce the volume of the stomach 3 in an endoscopic sleeve gastroplasty procedure. In this embodiment of the invention with the invaginating device 201 secured to the distal end 22 of the endoscope 24, and with the suture accommodating bore 124 of the cannula 119 of the suturing instrument 110 charged with a plurality of the sutures 125, and secured to the endoscope 24 by the clips 203, and with the distal end 115 of the outer sleeve 113 of the suturing instrument 110 terminating and engaged in the guide bore 222, and with the cannula 119 in the withdrawn state with the distal piercing tip 122 withdrawn into the cannula bore 117 of the outer sleeve 113, the suturing apparatus 200 is ready for use.

In the carrying out of an endoscopic sleeve gastroplasty procedure, the suturing apparatus 200 with the invaginating device 201 engaged with the distal end 22 of the endoscope 24, and the suturing instrument 110 secured to the endoscope 24 by the clips 203, is entered orally through the oesophagus into the stomach 3. As already described with reference to FIGS. 9 to 24 when describing the suturing of the front and rear walls 15 and 16 of the stomach 3 using the suturing apparatus 1, the endoscope 24 is maneuvered for aligning the suction chamber 215 of the invaginating device 201 with the first of the locations 17 at which an invaginated part 42 is to be invaginated, for example, the first one of the locations 17 on the inner side 13 of the front wall 15 of the stomach 3. The access opening 217 to the suction chamber 215 is brought into engagement with the location 17, and suction is applied to the suction chamber 215 through the instrument channel 26 of the endoscope 24 for forming the first one of the invaginated parts 42 of the pair thereof, see FIG. 43, on the front wall 15 of the stomach 3.

With the first invaginated part 42 retained in the suction chamber 215, the cannula 119 is urged distally from the withdrawn state by the operating level (not shown) of the handle (also not shown) through the distal end 115 of the outer sleeve 113 in order to urge the distal piercing tip 122 through the first invaginated part 42, see FIG. 43. With the distal piercing tip 122 extending on the distal side 79 of the first invaginated part 42, the foot switch of the suturing instrument 110 is operated to produce the activation signal, to which the microcontroller 139 is responsive for activating the linear actuator 138 to urge the pusher rod 135 distally through one of the predefined distances in order to discharge the first and distal most one of the anchor elements 54 of the distal most suture 125 from the suture accommodating bore 124 through the distal piercing tip 122, see FIG. 44. With the first of the anchor elements 54 located on the distal side 79 of the first of the invaginated parts 42, the cannula 119 is withdrawn from the first of the invaginated parts 42 into the withdrawn state with the suture thread 51 extending from the first one of the anchor elements 54 through the first one of the invaginated parts 42, see again FIG. 44. The suction on the suction chamber 215 is terminated thereby releasing the first of the invaginated parts 42 from the suction chamber 215.

The foot pedal 140 is again operated to produce another one of the activation signals, to which the microcontroller 139 is responsive to operate the linear actuator 138 to urge the pusher rod 135 further distally another one of the predefined distances in the suture accommodating bore 124 in order to discharge the cinch clip 57 of the distal most suture 125 in the suture accommodating bore 124 through the distal piercing tip 122 of the cannula 119 into the stomach 3, see again FIG. 44.

The endoscope 24 is again maneuvered to manoeuvre the invaginating device 201 to form the second one of the pair of invaginated parts 42 at the first of the locations 17 on the inner surface 13 of the rear wall 16 of the stomach 3. The suction chamber 215 is aligned with the location 17 of the rear wall 16 at which the second invaginated part 42 is to be formed, and the invaginating device 201 is urged towards the location 17 to bring the access opening 217 of the suction chamber 215 into engagement with the location 17. Suction is again applied to the suction chamber 215 to form the second one of the pair of invaginated parts 42.

With the second invaginated part 42 of the pair thereof retained in the suction chamber 215, the cannula 119 is again urged distally in the outer sleeve 113 to urge the distal piercing tip 122 through the second invaginated part 42. The foot pedal 140 is again operated to produce another one of the activation signals, to which the microcontroller 139 is responsive for operating the linear actuator 138 to urge the pusher rod 135 further distally a further one of the predefined distances in the suture accommodating bore 124 for discharging the second and now distal most one of the anchor elements 54 of the distal most suture 125 from the suture accommodating bore 124 through the distal piercing tip 122, to thereby deposit the second anchor element 54 of the suture 125 on the distal side 79 of the second invaginating part 42. The cannula 119 is again withdrawn from the second invaginated part 42 into the withdrawn state in the outer sleeve 113 with the suture thread 51 extending from the second one of the second anchor elements 54 through the second one of the invaginated parts 42, and with the loop 55 of the suture thread 51 of the suture 125 extending through the cinch clip 57. The suction in the suction chamber 215 is terminated, thereby releasing the second invaginated part 42 of the pair thereof from the suction chamber 215.

The next two invaginated parts of the next pair thereof to be formed at corresponding locations 17 on the front and rear walls 15 and 16, respectively, of the stomach 3 are again invaginated and the next suture 125, which is now the distal most suture 125 in the suture accommodating bore 124 is sequentially inserted into the next two invaginated parts 42 as the invaginated parts 42 are being formed, as described with reference to the first pair of the invaginated parts 42, and so on, until all of the invaginated parts 42 of the pairs thereof are formed at the respective locations 17 on the inner surface 13 of the front and rear walls 15 and 16 of the stomach 3 and the sutures 125 are inserted in the respective pairs of invaginated parts 42 as already described. Thereafter, a tightening instrument is inserted into the stomach through the instrument channel 27 of the endoscope 24, and in turn through the instrument bore 225 of the invaginating device 201 for tightening the sutures 125.

The tightening instrument is then operated as described above for sequentially tightening the sutures 125 for in turn drawing the corresponding pairs of invaginated parts 42 together to form the gastric sleeve 6.

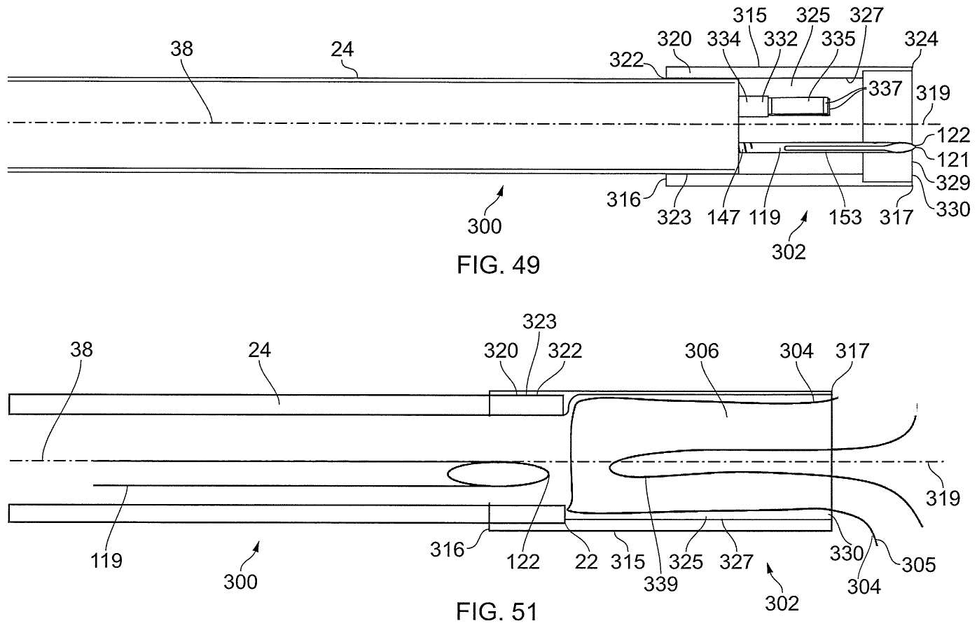

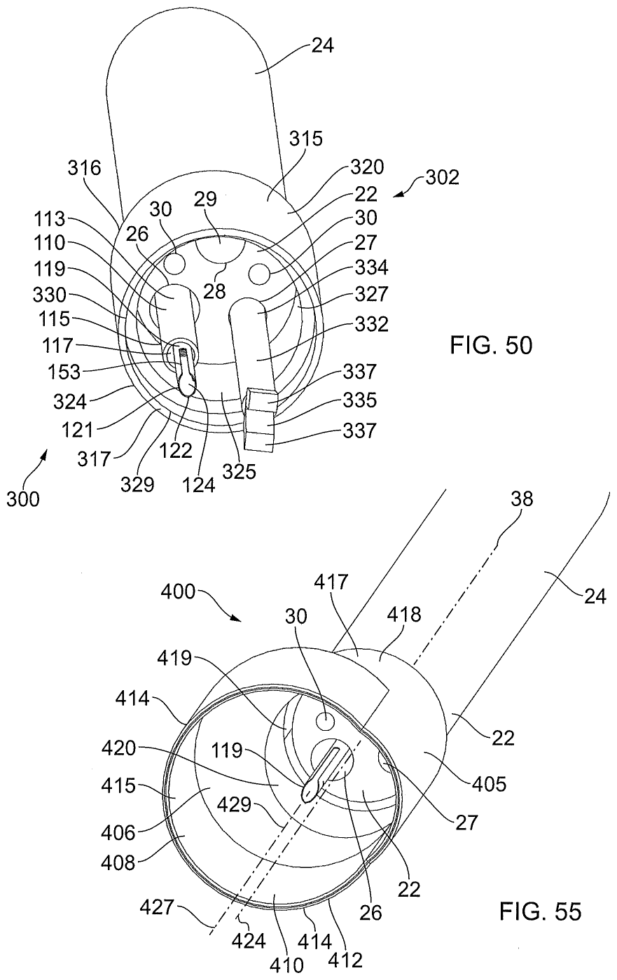

Referring now to FIGS. 47 to 54, there is illustrated a suturing apparatus also according to the invention and indicated generally by the reference numeral 300 comprising an access device, in this embodiment of the invention a dual channel endoscope 24, and an invaginating device according to another embodiment of the invention indicated generally by the reference numeral 302. The endoscope 24 is similar to the endoscope 24 described with reference to FIGS. 1 to 7 and similar components are identified by the same reference numerals. In this embodiment of the invention the invaginating device 302 is particularly suitable for invaginating a portion 304 of a wall 305 of a lumen, vessel or organ to form an invaginated part 306, see FIG. 51, for suturing to another similarly formed invaginated part (not shown) of the wall of the lumen, vessel or organ. For example, the invaginating device 302 is particularly suitable for use in an endoscopic sleeve gastroplasty procedure for forming a gastric sleeve extending through the stomach of a subject as already described with reference to the method of FIGS. 1 to 24, and also with reference to FIGS. 38 to 46. However, in this embodiment of the invention only the portion 304 of the wall 305 of the stomach or other lumen, vessel or organ is illustrated.

Turning now to the invaginating device 302, the invaginating device 302 comprises a tubular body member 315 of circular transverse cross-section of a transparent polymer material. The body member 315 extends from a proximal end 316 to a distal end 317 and defines a main central axis 319 extending therethrough from the proximal end 316 to the distal end 317. A connector 320 adjacent the proximal end 316 releasably secures the body member 315 to the endoscope 24 adjacent the distal end 22 thereof. In this embodiment of the invention the connector 320 comprises a socket 322 formed by a bore 323 of circular transverse cross-section extending axially into the body member 315 from the proximal end 316 thereof. The bore 323 defining the socket 322 defines a central longitudinally extending axis which in this embodiment of the invention coincides with the main central axis 319 of the body member 315, and also coincides with a central axis 38 of the endoscope 24. The distal end 317 of the body member 315 terminates in a distal end face 324, extending transversely of the main central axis 319 defined by the body member 315, and to which the main central axis 319 extends perpendicularly.

A suction chamber 325 extends axially into the body member 315 from the distal end face 324 thereof for invaginating the portion 304 of the wall 305 of the lumen, vessel or organ to form the invaginated part 306 thereof, when suction is applied to the suction chamber 325, as will be described below. The suction chamber 325 is formed by a bore 327 of circular transverse cross-section extending axially into the body member 315 from the distal end face 324 thereof. The bore 327 forming the suction chamber 325 defines a central longitudinally extending axis which coincides with the main central axis 319 defined by the body member 315. An inner edge 329 of the distal end face 324 of the body member 315 defined by the bore 327 defines an access opening 330 to the suction chamber 325 for accommodating the portion 304 of the wall 305 into the suction chamber 325 to form the invaginated part 306. The access opening 330 defines a plane extending perpendicularly to the main central axis 319 of the body member 315, which coincides with the distal end face 324 of the body member 315.

In this embodiment of the invention the bore 327 forming the suction chamber 325 terminates in and communicates with the bore 323 forming the socket 322 of the connector 320, so that the suction chamber 325 is essentially defined by the bore 327 and the distal end 22 of the endoscope 24 when the endoscope 24 is engaged in the socket 322. Thereby, the suction chamber 325 communicates directly with the instrument channels 26 and 27 of the endoscope 24, so that the suction chamber 325 communicates directly with the instrument channels 26 and 27 of the endoscope 24.

In this embodiment of the invention the instrument channel 27 of the endoscope 24 is configured for applying suction to the suction chamber 325 for suctioning and in turn invaginating the part 306 into the suction chamber 325, so that when suction is applied to the instrument channel 27, the portion 304 of the wall 305 of the lumen, vessel or organ is sucked into the suction chamber 325 to form the invaginated part 306. The instrument channel 26 of the endoscope 24 is configured for accommodating a suturing instrument, for example, the suturing instrument 8 described with reference to FIG. 9, or the suturing instrument 110 described with reference to FIGS. 31 to 37 into the suction chamber 325 for inserting a suture, for example, the suture 50 or the suture 125 into the invaginated part 306 while the invaginated part 306 is retained in the suction chamber 325 by the suction applied thereto through the instrument channel 27, in a similar manner as already described.

As well was being adapted for applying suction to the suction chamber 325, the instrument channel 27 of the endoscope 24 is also adapted for accommodating a tightening instrument, for example, a tightening instrument 332 for tightening the suture 50 or the suture 125 once the suture has been inserted into a corresponding pair of invaginated parts 306 on respective portions 304 of the wall of the lumen, vessel or organ which are to be sutured together. In this embodiment of the invention the tightening instrument 332 comprises an elongated tubular member 334 configured to extend through the instrument channel 27 of the endoscope 24. The tubular member 334 terminates in a grabber 335 comprising a pair of jaws 337, which are operable by an operating member (not shown) extending internally through the tubular member 334. The operating member (not shown) extends through the tubular member 334 to the proximal end thereof and terminates in an operating element for in turn operating the jaws 337 between an open state and a closed state for gripping the loops 55 of the sutures 50 or 125 for tightening thereof. Such grabbers will be well known to those skilled in the art. Once the suture 50 or 125 has been inserted in the two invaginated parts 306 of the corresponding pair thereof, the grabber 335 is operated to grab the loop 55 of the suture 50 or 125, and the tubular member 334 of the tightening instrument 332 is drawn into and through the instrument channel 27 of the endoscope 24 until the cinch clip 57 engages the distal end 22 of the endoscope 24 so that further pulling of the loop 55 into the instrument channel 27 of the endoscope 24 results in the cinch clip 57 being urged along the loop 55 towards the anchor elements 54 for in turn reducing the effective length of the suture thread 51 of the suture 50 or 125 for in turn drawing the invaginated parts 306 together as already described with reference to FIGS. 1 to 24 and FIGS. 25 to 29.

In use, suturing of two portions of the wall of a lumen, vessel or organ together using the suturing apparatus 300 comprising the endoscope 24, the invaginating device 302 and either of the suturing instruments 8 or 110, is substantially similar to that already described with reference to FIGS. 1 to 24. The invaginating device 302 is secured to the endoscope 24 adjacent the distal end 22 thereof, by engaging the distal end 22 of the endoscope 24 in the socket 322 of the invaginating device 302. The endoscope 24 with the invaginating device 302 secured to the distal end 22 thereof is entered into the lumen, vessel or organ, for example, the stomach, orally through the oesophagus, if it is desired to use the suturing apparatus 300 in an endoscopic sleeve gastroplasty procedure to form a sleeve extending through the stomach of the subject. When entering the endoscope 24 into the stomach of the subject, the endoscope 24 is entered without the suturing instrument 8 or 110 or the tightening instrument 332.

When the endoscope 24 has been inserted into the stomach with the invaginating device 302 located thereon, the suturing instrument, for example, the suturing instrument 110 appropriately charged with sutures 125 is inserted through the instrument channel 26 of the endoscope 24 until the distal end 115 of the outer sleeve 113 of the suturing instrument 110 has just about reached the distal end 22 of the endoscope 24. During insertion of the suturing instrument 110 through the instrument channel 26, the cannula 119 is withdrawn into the outer sleeve 113, with the distal piercing tip 122 thereof in the withdrawn state located within the outer sleeve 113. Additionally, the suturing instrument is inserted into the instrument channel 26 so that the distal end 115 of the outer sleeve 113 thereof, terminates adjacent the distal end 22 of the instrument channel 26, but within the instrument channel 26.

The endoscope 24 is then maneuvered in the stomach to urge the access opening 330 to the suction chamber 325 into engagement with the portion 304 of the wall 305 of the stomach which is to be invaginated. With the access opening 330 to the suction chamber 325 engaging the portion 304 at which the invaginated part 306 is to be formed, suction is applied to the instrument channel 27 of the endoscope 24 for in turn sucking the portion 304 of the wall 305 into the suction chamber 325 to form the invaginated part 306. In this embodiment of the invention the wall 305 adjacent the portion 304 to be invaginated is drawn into the suction chamber 325 as illustrated in FIGS. 51 to 54. This, thus, enables one of the anchor elements 54 of the suture 125 to be deposited on the outer surface 339 of the wall 305 of the stomach.

With the invaginated part 306 formed and retained in the suction chamber 325 by the suction applied through the instrument channel 27, the cannula 119 of the suturing instrument 110 is urged distally through the distal end 115 of the outer sleeve 113, and the distal piercing tip 122 of the cannula 119 is urged through the invaginated part 306 in the suction chamber 325 until the distal piercing tip 122 extends through the wall 305, and in turn through the outer surface 339 of the invaginated part 306 within the suction chamber 325. However, in order to avoid piercing or puncturing of other lumens, vessels or organs in a cavity of the subject within which the stomach is located, the distal travel of the cannula 119 outwardly from the distal end 115 of the outer sleeve 113 is controlled, so that when the cannula 119 has been urged distally outwardly through the distal end 115 of the outer sleeve 113 to its maximum distal travel, the distal piercing tip 112 does not extend distally outwardly through the access opening 330, and is located within the suction chamber 325.

Once the distal piercing tip 122 of the cannula 119 has pierced through the outer surface 339 of the wall 305 of the invaginated part 306, the foot pedal 140 of the pedal operated switch of the suturing instrument 110 is operated to produce one of the activating signals, to in turn activate the microcontroller 139 to operate the linear motor 138, to in turn urge the pusher rod 135 of the suturing instrument 110 through one of the predefined distances, for in turn urging the distal most anchor element 54 of the distal most suture 125 in the cannula 119 through the distal piercing tip 122, so that the anchor element 54 is located adjacent the outer surface 339 of the wall 305 of the invaginated part 306. The cannula 119 of the suturing instrument 110 is withdrawn through the invaginated part 306 and is withdrawn into the outer sleeve 113, thereby leaving the adjacent portion of the suture thread 51 extending from the anchor element 54 through the wall 305 of the stomach. The suction is then removed from the instrument channel 27 to thereby release the invaginated part 306 from the suction chamber 325. Thereafter, operation of the suturing apparatus 300 is similar to the operation of the invaginating device 201 and the suturing instrument 110 already described with reference to FIGS. 31 to 37, and FIGS. 38 to 46. The suturing instrument 110 is again operated to discharge the cinch clip 57 into the stomach, and the endoscope 24 is maneuvered for maneuvering the access opening 330 to the suction chamber 325 to engage the portion of the wall of the stomach at which a second invaginated part 306 of a pair of invaginated parts thereof is to be formed, and so operation of the suturing apparatus 300 comprising the endoscope 24, the invaginating device 302 and the suturing instrument 110 continues in suturing the front and rear walls 15 and 16 of the stomach 3 in the endoscopic sleeve gastroplasty procedure.

It is envisaged in embodiments of the invention where tightening of the sutures is deferred until after all the sutures have been inserted in the invaginated parts, the suturing instrument may be withdrawn through the instrument channel 26, and instead of inserting the tightening instrument 332 into the stomach through the instrument channel 27, the tightening instrument 332 may be inserted into the stomach through the instrument channel 26 from which the suturing instrument 110 has been removed.

It is also envisaged that in some embodiments of the invention the endoscope 24 instead of comprising a dual channel endoscope may be a single channel endoscope, in which the single channel would be used for applying suction to the suction chamber 325 and also for accommodating the suturing instrument simultaneously into the suction chamber 325. It is envisaged that there would be sufficient space in the instrument channel of a single instrument channel endoscope between the suturing instrument and the wall defining the instrument channel to accommodate sufficient suction for suctioning the invaginated part 306 and retaining the invaginated part 306 in the suction chamber 325 during insertion of the corresponding legs 56 of the suture thread 51 of the suture and the corresponding anchor element 54 through the invaginated part 306.

Indeed, in some embodiments of the invention it is envisaged that suction may be applied to the suction chamber through a conduit or an elongated tubular member releasably secured to and extending longitudinally along and externally of the endoscope or other access device by the clips 203 described with reference to FIGS. 38 to 46 or any other suitable securing means. It is also envisaged that the suturing instrument may be releasably secured to and extend longitudinally along and externally of the endoscope or other access device, and in turn into the suctioning chamber 325, by the clips 203 or by any other securing means.

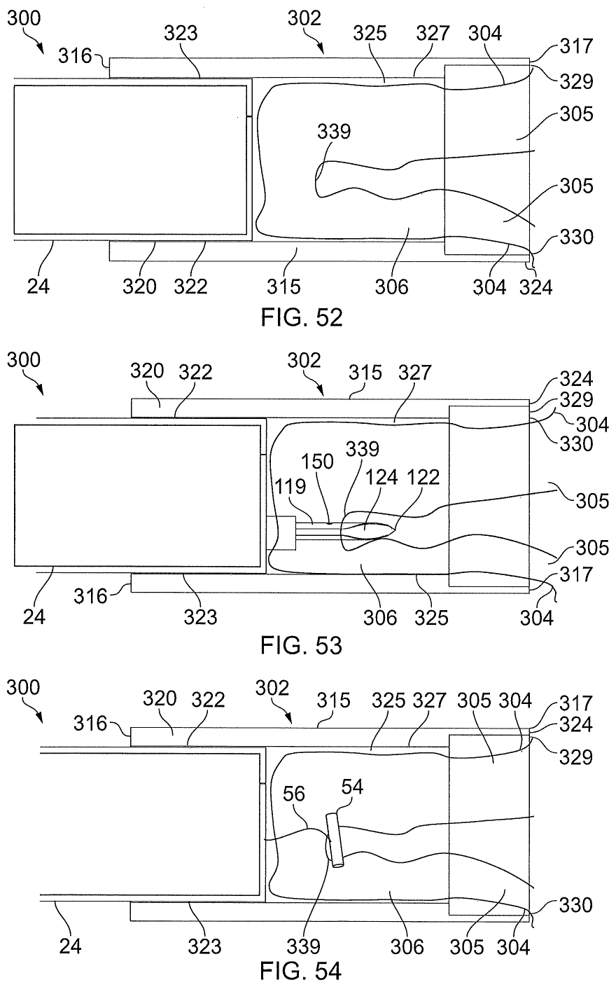

A particularly important advantage of the invaginating device 302 according to this embodiment of the invention is that the suction chamber 325 can be of any suitable axial length such that the invaginated part 306 of the wall 305 of the stomach or other lumen, vessel or organ can be drawn into the suction chamber 325 with the outer surface 339 of the wall 305 of the stomach of the invaginated part 306, which is located adjacent the proximal end of the suction chamber 325, well-spaced apart inwardly into the suction chamber 325 from the access opening 330 thereto. Thereby, the anchor element 54 and the adjacent part of the suture thread 51 can be inserted through the invaginated part 306 through the outer surface 339 of the wall 305 of the invaginated part 306, as illustrated in FIGS. 52 to 54, without any risk of the piercing tip 122 of the cannula 119 extending to or through the access opening 330. This, thus, avoids any risk of the piercing tip 122 of the cannula 119 piercing or engaging a lumen, vessel or organ or other tissue in the abdominal cavity external of the stomach. Needless to say, this advantage also applied to the invaginating device 302 in cases where the invaginating device 302 is used for carrying out suturing within any other lumen, vessel or organ. The advantage of being able to insert the anchor element 54 and the adjacent part of the suturing thread 51 through the wall of a lumen, vessel or organ and through the outer surface thereof, is that the suture gains a better and stronger grip on the part or parts of the lumen, vessel or organ to be sutured together.

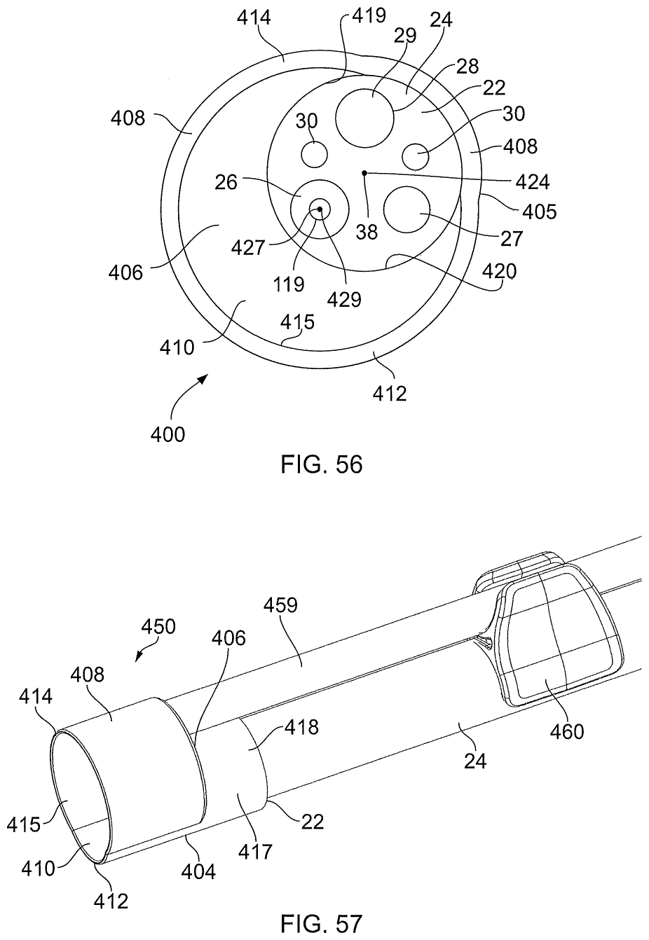

Referring now to FIGS. 55 and 56, there is illustrated an invaginating device according to a further embodiment of the invention indicated generally by the reference numeral 400. The invaginating device 400 is somewhat similar to the invaginating device 302 described with reference to FIGS. 47 to 54, and is adapted for securing to an access device, in this case the endoscope 24 adjacent a distal end 22 of the endoscope 24. The endoscope 24 is similar to the endoscope 24 described with reference to FIGS. 1 to 7, and similar components are identified by the same reference numerals. The invaginating device 400 is particularly suitable for invaginating a portion 304 of a wall 305 of a lumen, vessel or organ as illustrated in FIGS. 47 to 54, and then inserting a suture similar to the suture 125, see FIG. 8, into the invaginated part 306 as described with reference to the invaginating device 302 described with reference to FIGS. 47 to 54.

In this embodiment of the invention the invaginating device 400 comprises a body member 405 comprising an end wall 406 and a side wall 408 extending distally from the end wall 406 and defining with the end wall 406 a suction chamber 410 of substantially circular transverse cross-section. The side wall 408 terminates in a distal end 412 and defines a distally facing end face 414. The distal end 412 of the side wall 408 defines a main access opening 415 communicating with the suction chamber 410, so that when the end face 414 is engaged with a portion 304 of the wall 305 of a lumen, vessel or organ to be invaginated, on applying a vacuum to the suction chamber 410, the portion of the wall 305 is drawn into the suction chamber 410 to form the invaginated part 306 as illustrated in FIG. 52.

A connector 417 extends proximally from the end wall 406 of the body member 405 for releasably engaging the distal end 22 of the endoscope 24 with a friction grip fit for securing the invaginating device 400 to the endoscope 24. The connector 417 comprises a main sleeve 418 extending proximally from the end wall 406 and defining a main socket 419 of circular transverse cross-section for releasably, sealably and tightly engaging the distal end 22 of the endoscope 24 with the friction grip fit. A main communicating opening in this embodiment of the invention, a main communicating duct 420 extending through the end wall 406 communicates the suction chamber 410 with the main socket 419, for in turn communicating the channels of the endoscope 24 with the suction chamber 410. The main communicating duct 420 is of circular transverse cross-section and of diameter substantially similar to the diameter of the main socket 419. The main socket 419 defines a socket central axis 424, which when the connector 417 is engaged on the distal end 22 of the endoscope 24 coincides with a longitudinally extending central axis 38 of the endoscope 24 adjacent the distal end 22 thereof.

The suction chamber 410 defines a chamber central axis 427 which extends parallel to the socket central axis 424, but is offset from the socket central axis 424. The connector 417 is positioned relative to the suction chamber 410 so that the chamber central axis 427 is sufficiently offset from the socket central axis 424 that by selecting the angular orientation of the suction chamber 410 around the central axis 38 of the endoscope 24 when the connector 417 is being engaged on the distal end 22 of the endoscope 24, the chamber central axis 427 may be substantially centrally aligned with an instrument channel, for example, the instrument channel 26 of the endoscope 24, which is offset from the central axis 38 of the endoscope 24.

By engaging the invaginating device 400 on the endoscope 24 with the chamber central axis 427 of the suction chamber 410 substantially centrally aligned with the instrument channel 26, a cannula, such as the cannula 119 of the suturing instrument 110, see FIGS. 31 to 37, when introduced into the suction chamber 410 through the centrally offset instrument channel 26, the cannula 119 is substantially aligned with the chamber central axis 427. This enables the cannula 119 to pierce the invaginated part 306 drawn into the suction chamber 410 centrally in the suction chamber 410. As will be well known to those skilled in the art, while in some endoscopes, the instrument channel thereof extends centrally through the endoscope, with a central axis of the instrument channel coinciding with the central axis of the endoscope, in many endoscopes, the instrument channel is offset from the longitudinally extending central axis of the endoscope as in the endoscope 24. Thus, by correctly selecting the angular orientation of the suction chamber 410 around the central axis 38 of the endoscope 24 when the invaginating device 400 is being engaged with the endoscope 24, the chamber central axis 427 may be substantially centrally aligned with the instrument channel 26, and in general, may be aligned with the instrument channel 26, such that the chamber central axis 427 coincides with a longitudinally extending central axis 429 of the instrument channel 26 of the endoscope 24.

In use, to secure the invaginating device 400 to the endoscope 24 adjacent the distal end 22 thereof, initially the invaginating device 400 is offered up to the distal end 22 of the endoscope 24, and the invaginating device 400 is oriented around the central axis 38 of the endoscope 24 until the chamber central axis 427 substantially coincides with the central axis 429 of the instrument channel 26 through which it is intended to introduce the cannula 119 of the suturing instrument 110, so that the cannula 119 will extend centrally into the suction chamber 410 with the cannula 119 substantially coinciding with the chamber central axis 427. Once the invaginating device 400 is appropriately orientated angularly about the central axis 38 of the endoscope 24, the distal end 22 of the endoscope 24 is fully engaged into the main socket 419 of the connector 417 with the distal end 22 of the endoscope 24 tightly and sealably secured in the connector 417.

Thereafter, the endoscope 24 with the invaginating device 400 secured to the distal end 22 thereof is introduced into the lumen, vessel or organ into which suturing of the parts of the lumen, vessel or organ is to be carried out. The end face 414 of the side wall 408 is engaged with the portion 304 of the wall 305 of the lumen, vessel or organ which is to be invaginated, and a vacuum is applied to the suction chamber 410 through the instrument channel 27 of the endoscope 24 to thereby draw the portion 304 of the wall 305 into the suction chamber 410 to form the invaginated part 306.

With the vacuum still applied to the suction chamber 410, the cannula 119 of the suturing instrument 110 is entered through the instrument channel 26 of the endoscope 24 into the suction chamber 410, and in turn through the invaginated part 306 and through the outer surface 339 thereof. The distal most one of the anchoring elements 54 of the distal most one of the sutures 125 is then urged from the cannula 119, so that the anchor element 54 of the suture 125 is located on the outer surface 339 of the invaginated part 306 of the wall 305 of the lumen, vessel or organ as described in the embodiment of the invaginating device 302 and illustrated in FIGS. 53 and 54. The cannula 119 is then withdrawn into the instrument channel 26 of the endoscope 24 from the suction chamber 410, and in turn from the invaginated part 306 and the vacuum is released from the suction chamber 410, and so the invaginating and suturing of the invaginated parts continues as already described with reference to the suturing instrument 110.

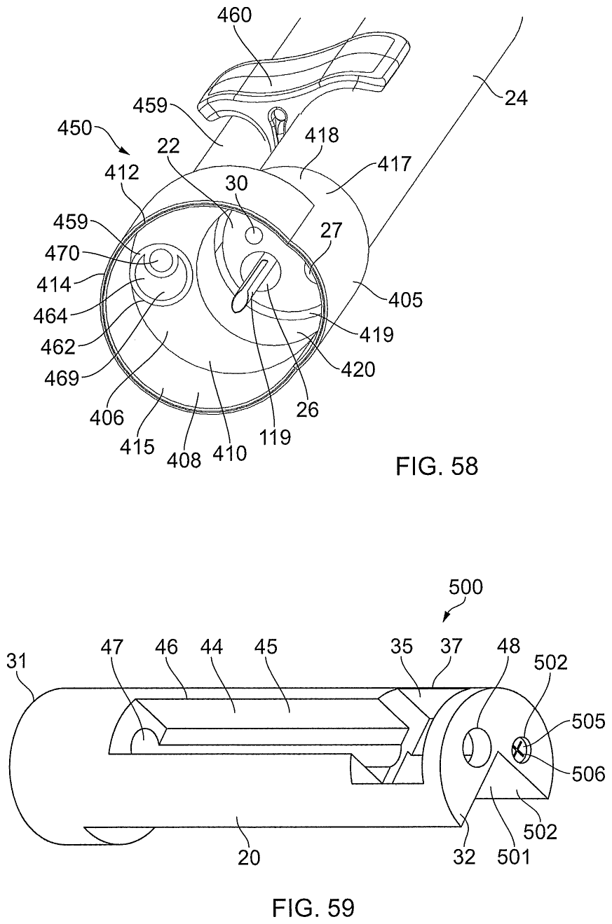

Referring now to FIGS. 57 and 58, there is illustrated an invaginating device according to another embodiment of the invention indicated generally by the reference numeral 450 releasably secured to the endoscope 24 adjacent the distal end 22 thereof. The endoscope 24 is similar to the endoscope 24 described with reference to FIGS. 1 to 7, and similar components are identified by the same reference numerals. The invaginating device 450 is substantially similar to the invaginating device 400 described with reference to FIGS. 55 and 56, and similar components are identified by the same reference numerals. In this embodiment of the invention the invaginating device 450 comprises a connector 417, which is similar to the connector 417 of the invaginating device 400, for releasably securing the invaginating device 450 to the distal end 22 of the endoscope 24.

The body member 405 is substantially similar to the body member 405 of the invaginating device 400, with the exception that the body member 405 of the invaginating device 450 is also adapted for connecting to an elongated tubular member 459 extending externally along and secured to the endoscope 24 by a plurality of clips 460 spaced apart along the endoscope 24, as will be described below. A secondary communicating opening 462 formed in the end wall 406 of the body member 405 is adapted to sealably engage the tubular member 459 with the suction chamber 410 communicating with a pair of elongated bores, namely, a main bore 464 and a secondary bore 465 extending through the tubular member 459.

The tubular member 459 is configured to either apply a vacuum to the suction chamber 410 for invaginating the portion 304 of the wall 305 of the lumen, vessel or organ to be invaginated, or for accommodating an instrument into the suction chamber 410, which may be required during the invagination of the portion 304 of the wall 305 of the lumen, vessel or organ, or which may be required during the suturing of the portions 304 of the wall 305 of the lumen, vessel or organ. In some embodiments of the invention the tubular member 459 may be adapted to accommodate an insufflating gas into the lumen, vessel or organ during movement of the invaginating device 450 from one site to another site of the portions 304 of the wall 305 of the lumen, vessel or organ at which the respective invaginated parts 306 of pairs thereof are to be formed. The tubular member 459 may also be used for delivering insufflating gas to the lumen, vessel or organ during the suturing of the invaginated parts 306 together. A secondary sleeve (not shown) extending proximally from the end wall 406 of the body member 305 around the secondary communicating opening 462 defines a secondary socket (also not shown) of circular transverse cross-section for releasably, tightly and sealably engaging the tubular member 459 with a tight friction grip fit.

Returning now to the tubular member 459, in some cases, the main bore 464 may be used for delivering insufflating gas therethrough, and the secondary bore 465 may be used for monitoring the static pressure in the lumen, vessel or organ remotely when the suction chamber 410 is not being used for forming the invaginated part or for retaining the invaginated part therein, in other words, when the suction chamber 410 is not being used for suctioning. It is also envisaged that during the invaginating part of the suturing process, the main bore 464 of the tubular member 459 may be utilised to apply the vacuum to the suction chamber 410, and on completion of the invaginating of the portions 304, the main bore 464 may be utilised to deliver insufflating gas to the lumen, vessel or organ while the secondary bore 465 may be used to remotely monitor the static pressure in the lumen, vessel or organ. It is also envisaged that in some embodiments of the invention a tubular member similar to the tubular member 459 but with the exception that the tubular member would include only a single bore of circular transverse cross-section, may be used for accommodating an instrument into the suction chamber 410.

The clips 460 are similar to the clips 203 described with reference to the embodiment of FIGS. 38 to 46, and are adapted to resiliently and releasably engage the endoscope 24 and to also resiliently and releasably engage the tubular member 459. The tubular member 459 is secured to the endoscope 24 by the clips 460 along the entire length of the endoscope 24, so that along with the proximal end (not shown) of the endoscope 24, a proximal end (also not shown) of the tubular member 459 extends externally of the subject. A loop tether (not shown) retains the clips 460 connected together as described with reference to FIGS. 38 to 46.

Otherwise, the invaginating device 450 is similar to the invaginating device 400 and its use is likewise similar.

An advantage of the invaginating device 450 is that by virtue of the fact that the body member 405 is adapted to receive the tubular member 459, the provision of the tubular member 459 frees up one of the channels of the endoscope, for example, by utilising the tubular member 459 to apply a vacuum to the suction chamber 410, the channel of the endoscope which would otherwise be required for applying a vacuum to the suction chamber 410 is free for another use. Alternatively, by using the tubular member 459 to accommodate an instrument into the lumen, vessel or organ, the instrument channel of the endoscope may be used simultaneously for accommodating another instrument into the lumen, vessel or organ, or the instrument channel of the endoscope may be used for any other purpose. Additionally, if the tubular member 459 is used as an insufflating channel, a channel in the endoscope, which would otherwise be used for insufflating the lumen, vessel or organ, is freed up for other uses.

An advantage of the invaginating devices described with reference to FIGS. 47 to 54, is that, it is believed that better sealing of the suction chamber is achieved between the body member of the invaginating device and the wall of the lumen, vessel or organ around the access opening when the access opening is formed in the distal end face of the body member. Additionally, within reason, there is no limit to the depth of the suction chamber, since the depth of the suction chamber is determined by the axial length of the portion of the body member forming the suction chamber. Accordingly, the extra depth of the suction chamber, and the enhanced sealing of the suction chamber around the access opening thereto with the wall of the lumen, vessel or organ, enables a relatively large part of the wall of the lumen, vessel or organ to be sucked into the suction chamber to the extent that the invaginated part sucked into the suction chamber includes a portion of the outer surface of the wall of the lumen, vessel or organ. This, thus, enables the cannula to pierce through the part of the wall of the lumen, vessel or organ forming the invaginated part to and through the outer surface of the wall in the invaginated part located in the suction chamber. This, thus, allows the corresponding anchor element of the suture to be deposited on the outer surface of the invaginated part. Thereby when two invaginated parts of the lumen, vessel or organ are drawn together by the suture, the invaginated parts are more strongly retained together. A further advantage of drawing the part to be sutured into the suction chamber, such that the outer surface of the wall of the lumen, vessel or organ forms a part of the invaginated part, enables the anchor element of the suture to be discharged by the cannula on the outer surface of the wall of the lumen, vessel or organ, without the distal piercing tip of the cannula extending outwardly of the suction chamber through the access opening thereto. This, thus, avoids any danger of another lumen, vessel or organ located in the cavity in which the lumen, vessel or organ being sutured is located, being pierced, punctured or otherwise damaged by the distal piercing tip of the cannula.

Referring now to FIG. 59 there is illustrated an invaginating device according to another embodiment of the invention indicated generally by the reference numeral 500. The invaginating device 500 is substantially similar to the invaginating device 10 described with reference to FIGS. 1 to 7, and similar components are identified by the same reference numerals. In this embodiment of the invention the only difference between the invaginating device 500 and the invaginating device 10 is that a viewing aperture 501 is provided in the body member 20, and an additional instrument bore 502 extends through the body member 20 from the socket 34 to and through the distal end 32 of the body member 20. The viewing aperture 501 is formed by an elongated viewing recess 504 formed into the body member 20 extending from and communicating with the socket 34 to the distal end 32 of the body member 20. The viewing recess 504 communicates with the vision channel 28 of the endoscope 24 through the socket 34 for facilitating viewing of the interior of the stomach, or other lumen, vessel or organ through the vision channel 28 of the endoscope 24. The additional instrument bore 502 is similar to the instrument bore 225 of the invaginating device 201 described with reference to FIGS. 38 to 46, and may or may not extend through the suction chamber 35. The instrument bore 502 is closed adjacent its distal end by a sealing membrane 505 for minimising air being drawn through the instrument channel 27 of the endoscope while suction is applied to the suction chamber 35 in cases where the instrument bore 502 passes through the suction chamber 35. A cross slit 506 is formed in the membrane 505 to accommodate an instrument therethrough, and to allow the membrane 505 to seal against the instrument.

Otherwise, the invaginating device 500 and its use is similar to the invaginating device 10 described with reference to FIGS. 1 to 24.

While the urging means for urging the pusher rod 135 of the suturing instrument 110 through the predefined distances has been described as comprising a linear actuator, any other suitable actuator may be provided, and such actuators may comprise either linear or rotary motor. When provided with rotary motors, a transmission system between the rotary motor and the pusher rod would be provided for converting rotational motion of the rotary motor into linear motion of the pusher rod. Such a transmission system may, for example, comprise a rack and pinion drive or a worm and gear drive. It will also be appreciated that instead of the actuator being electrically powered, the actuator may be pneumatically powered. Further, it will be appreciated that the linear motor or indeed a rotary motor of the linear actuator may act on the pusher rod through a ratchet system, whereby, the ratchet would sequentially engage spaced apart projections on the pusher rod for urging the pusher rod through the predefined distances.

It will also be appreciated that any number of sutures may be located in the suture accommodating bore of the cannula 119 of the suturing instrument 110, and the number of sutures which may be located in the suture accommodating bore of the cannula would be limited only by the length of the cannula, and the length of the anchor elements and the length of the cinch clips.

While the suturing instruments have been described as comprising sutures of a specific type, it will be readily apparent to those skilled in the art that any other suitable sutures may be used in conjunction with the suturing instruments. It will be appreciated that the sutures may comprise different types of anchor bars or anchor elements, and different types of cinch clips to those described, and further, it will be appreciated that while it is desirable, it is not essential that the anchor bars of each suture be of the same length, and furthermore, it will be appreciated that the cinch clips of each suture need not be the same length as the anchor bars thereof.

In cases where the anchor bars of each suture are of different lengths, and the cinch clip of each suture is of a different length to the anchor bars, the microcontroller of the suturing instrument 110 would be programmed to operate the linear actuator to urge the pusher rod 135 appropriate first, second or third predefined distance depending on the lengths of the anchor bars and/or cinch clips for discharging the relevant ones of the anchor bars and/or the cinch clip, as the case may be, and the distance through which the pusher rod 135 would be urged by the actuator would correspond to the length of the relevant one of the anchor bars or the cinch clip to be discharged from the suture accommodating bore through the distal piercing tip 122 of the cannula. Needless to say, it is not essential that the anchor bars be of circular transverse cross-section, all that is required is that they be of cross-sectional dimensions suitable for sliding in the suture accommodating bore of the cannula. Needless to say, other types of cinch clips may be used in conjunction with the sutures other than a cinch clip of the type illustrated in FIG. 8.

While the cannula and the outer sleeve of the suturing instruments 110 according to the invention have been described as being of circular transverse cross-section, it will be readily apparent to those skilled in the art that the outer sleeve and the cannula of the suturing instruments may be of any suitable transverse cross-section, for example, the cannula and the outer sleeve may be of square, rectangular, triangular, hexagonal, octagonal, or any other suitable transverse cross-section. Further, it is envisaged that the transverse cross-section of the cannula may be different to that of the outer sleeve. Additionally, in some embodiments of the invention it is envisaged that the cannula may be keyed in the outer sleeve in order to prevent rotation of the cannula in the outer sleeve relative to the outer sleeve.

Additionally, it is envisaged that in some embodiments of the invention the suturing instrument 110 may be provided without an outer sleeve.

It is also envisaged that in some embodiments of the invention a sensing means may be provided adjacent the distal end of the cannula of the suturing instrument 110, which would be capable of identifying and distinguishing between the cinch clip and the anchor elements of each suture, and may also be configured to distinguish between the two anchor elements of each suture. Such a sensing means would produce a signal indicative of the one of the cinch clip and the anchor elements adjacent the distal end of the cannula and next to be discharged therethrough. The signals from the sensing means would be read by the microcontroller, which would then control the linear actuator to urge the pusher rod distally the appropriate predefined distance for discharging the relevant one of the cinch clip or anchor element adjacent the distal end of the cannula.

It is also envisaged that the microcontroller 139 may be pre-programmed to operate the linear actuator 138 through three predefined distances, for example, a first predefined distance to discharge one of the anchor bars of each suture from the suture accommodating bore of the cannula, a second predefined distance to discharge the other one of the anchor bars of each suture from the suture accommodating bore of the cannula, and a third predefined distance to discharge the cinch clip of each suture from the suture accommodating bore of the cannula. It is envisaged that the sequence of the first, second and third predefined distances through which the linear actuator 138 would be operated would be selectable, and would be selectable to match the sequence in which the anchor bars and the cinch clips of the respective sutures are located in the suture accommodating bore of the cannula. It is also envisaged that the lengths of the first, second and third predefined distances would be such to match the lengths of the respective anchor elements and the cinch clip, and would be selectable.

While the suturing instrument 110 has been described for use in an endoscopic sleeve gastroplasty procedure, it will be readily apparent to those skilled in the art that the suturing instruments 110 according to the invention may be used for suturing any two portions of a lumen, vessel or organ together either internally in or externally of the lumen, vessel or organ.

It is also envisaged that as well as the suturing instruments being entered into the lumen, vessel or organ to be sutured through the instrument channel of an endoscope, the suturing instruments may be entered into a lumen, vessel or organ or indeed a cavity in which suturing is to be carried out through the instrument channel of any other suitable access device, for example, a laparoscope, colonoscope, or any other suitable or appropriate access devices.

While the parts to be invaginated have been described as being invaginated by a multiband ligator, any other suitable invaginating means for forming invaginated parts may be used, for example, a grabber, which would grab a part of the portion of the lumen, vessel or organ to be sutured and would pull the part inwardly, or outwardly as the case may be from the wall of the subject, and while the part is grabbed, the cannula would be operated to pierce through the grabbed invaginated part, and the distal most one of the anchor bars of the distal most suture in the suture accommodating bore would be dispensed on the distal side of the grabbed invaginated part. Once the cannula has been withdrawn from the grabbed invaginated part, the grabber would release the invaginated part.

While the suturing instrument has been described for use in an endoscopic sleeve gastroplasty procedure, it will be readily apparent to those skilled in the art that the suturing instruments according to the invention may be used for suturing any two portions of a lumen, vessel or organ together either internally or externally of the lumen, vessel or organ.

It is envisaged that in some embodiments of the invention the pusher element of the suturing instrument 8 may be provided without the hook 70. In which case the pusher element 69 would merely terminate directly in the abutment end 71 for urging the anchor elements 54 sequentially out of the anchor element accommodating chamber 73. In cases where the pusher element 69 of the suturing instrument 8 is not provided without a hook 70, a separate suture tightening device discussed above would be provided for tightening the sutures. The suture tightening device would typically comprise a flexible sleeve terminating in a distal end abutment face similar to the outer sleeve of the suturing instrument 8. A hook similar to the hook 70 of the suturing instrument 8 or a grabber for grabbing and gripping the loops of the sutures would be located at the distal end of an elongated operating rod, and with the operating rod would be slideable within the sleeve inwardly and outwardly of the distal end of the sleeve. The sleeve of such a device would be entered through the endoscope or other access device into the stomach or other lumen, vessel or organ, so that by sequentially engaging or gripping the loops 55 of the sutures 50 with the hook or grabber, and urging the hook or grabber inwardly into the sleeve, with the cinch clip 57 of each suture 50 abutting the distal end abutment face of the sleeve, the cinch clip would be urged along the loop of the suture for tightening the suture and in turn urging the corresponding invaginated parts 42 tightly together.

It will also be appreciated that other suitable suturing instruments may be used in conjunction with any of the invaginating devices according to the invention without departing from the scope of the invention.

It is envisaged that in the embodiment of the invaginating devices 500 that since a viewing aperture is provided which communicates with the lens of the vision channel of the endoscope, the material of the invaginating devices 500 need not be of a transparent material. The material may be of a translucent or an opaque material. Additionally, while the viewing aperture 501 in the invaginating device 500 has been described as an elongated viewing recess, any other suitable viewing aperture may be formed in the body member of the invaginating device 500, and also in the other invaginating devices, which would communicate with the vision channel of an endoscope, in order to permit viewing of the interior or exterior, as the case may be, of the lumen, vessel or organ.

While the invaginating devices 10, 201, 302, 400, 450 and 500 have been described as being of cylindrical or partly cylindrical outer shape, it is envisaged that the invaginating devices 10, 201, 302, 400, 450 and 500 may be of any other suitable outer shape.

While in the various embodiments of the methods described, specific numbers of pairs of invaginated parts have been described at the respective locations 17 of the portions of the lumen, vessel or organ to be sutured together, it will be appreciated that any appropriate number of invaginated parts may be formed. For example, in the stomachs illustrated in FIGS. 10 to 15 and 28 and 29, while five pairs of invaginated parts have been illustrated, any number of pairs of invaginated parts may be formed along the line 19 along which the front and rear walls of the stomach are to be joined together to form the gastric sleeve 6. In general, the number of invaginated parts required along the line 19 will depend on the size of the stomach and the length of the line 19 along which the front and rear walls of the stomach are to be secured together and the knowledge and experience of the surgeon.

It will also be appreciated that the methods according to the invention may be used for everting a polyp in a wall of a lumen, vessel or organ externally to the outer side of the wall of the lumen, vessel or organ. In which case, pairs of invaginated parts would be formed on the inner side of the wall of the lumen, vessel or organ on opposite sides of the polyp at spaced apart intervals along the polyp by drawing the parts to be invaginated inwardly into the lumen, vessel or organ. The pairs of invaginated parts on the respective opposite sides of the polyp would then be drawn together by respective sutures, thereby everting the polyp to the outer side of the wall of the lumen, vessel or organ. The polyp could then be removed externally from the outer side of the wall of the lumen, vessel or organ, by other suitable minimal invasive surgeries, for example, laparoscopic surgery.

While some of the invaginating devices according to the invention have been described as having a distal instrument accommodating bore extending from the suction chamber to and through the distal end of the body member, it is envisaged that in some embodiments of the invention some or all of the invaginating devices will be provided without the distal instrument accommodating bore. This may be the case in embodiments of the invention where the suturing instrument is provided solely for inserting the sutures into the respective pairs of the invaginated parts 42 of the lumen, vessel or organ, and the suturing instrument is not used to tighten the sutures. In such cases, it is envisaged that a separate suture tightening instrument, for example, of the type described herein, would be used for tightening the sutures. In such cases, in general, on completion of the insertion of the sutures in the respective pairs of the invaginated parts, the endoscope would be withdrawn from the lumen, vessel or organ of the subject, the invaginating device would be removed, and the endoscope would be re-entered into the lumen, vessel or organ of the subject. The suture tightening instrument would then be passed through the instrument channel of the endoscope into the lumen, vessel or organ for tightening the sutures.

Alternatively, it is envisaged that where an invaginating device is provided with a distal instrument accommodating bore, on completion of insertion of the sutures into the respective pairs of the invaginated parts, the suturing instrument would be withdrawn through the instrument channel of the endoscope, and the suture tightening instrument would then be passed through the instrument channel of the endoscope into the lumen, vessel or organ through the instrument passageway and the distal instrument accommodating bore of the invaginating device with the distal end of the outer sleeve of the suture tightening instrument extending distally through the distal instrument accommodating bore of the invaginating device for tightening the sutures.

It is also envisaged that in some embodiments of the invention where either of the methods according to the invention are being used for carrying out an endoscopic sleeve gastroplasty procedure, as well as suturing the front and rear walls of the stomach to form the gastric sleeve along the line 19 of the sutures, a second line of sutures, which would be substantially parallel to and spaced apart from the line 19 of sutures forming the gastric sleeve, could be provided for securing the front and rear wall of the stomach together at a lower level in the stomach to that of the line 19 of sutures forming the gastric sleeve.

While the body members of the invaginating devices have been described as comprising a flexible material, it is envisaged that in some embodiments of the invention the flexibility of the material of the body member of the invaginating devices would be such that the body member would have a degree of resilience, and/or would be at least a self-supporting material in order to maintain the shape of the suction chamber, the instrument passageway and the socket. However, the material of the body member of the invaginating devices would be such as to permit bending of the invaginating device along the main longitudinal axis of the body member in order to enable the body member to pass around bends in lumens, vessels and organs, and to be maneuverable within the lumen, vessel or organ.

In the embodiments of the invention in which the suturing instrument is described as being releasably secured to the access device, for example, the endoscope, with the suturing instrument extending longitudinally along the access device and externally thereof, it is envisaged that in some embodiments of the invention the suturing instrument, instead could be provided to the invaginating device through an instrument channel of the access device or the endoscope, and a communicating conduit or an elongated tubular member could be secured releasably or otherwise to the access device or endoscope to extend longitudinally along and externally of the access device or endoscope, and the invaginating device would be configured to engage the communicating conduit or the tubular member with the suction chamber communicating with the communicating conduit or the tubular member, so that suction could be applied to the suction chamber through the conduit or the tubular member, rather than through an instrument channel of the endoscope or other access device. Such an arrangement would enable any of the invaginating devices with such an arrangement for connecting to an external communicating conduit or tubular member to be used with a single channel access device or endoscope.

It is also envisaged that the pusher rod 135 of the suturing instrument 110 may be provided with a hook adjacent its distal end for tightening the sutures.

While the access devices have been described as comprising endoscopes, it will be readily apparent to those skilled in the art that any other suitable access devices may be provided. It will also be appreciated that while the endoscope has been described as comprising an endoscope having a specific arrangement of channels, it will be readily apparent to those skilled in the art that any other suitable endoscope may be used. Needless to say, while the endoscope has been described as comprising a dual instrument channel endoscope, in many embodiments of the invention a single instrument channel endoscope would be sufficient.

1. An invaginating device for invaginating tissue and/or a part of a wall of a lumen, vessel or organ in the body of a human or animal subject, the invaginating device comprising:
a body member,
the body member comprising a connector adapted for coupling the body member to an access device adjacent a distal end thereof, the body member having
a suction chamber therein for invaginating the tissue and/or the part of the wall of the lumen, vessel or organ, and
an access opening to the suction chamber for accommodating the tissue and/or the part of the wall of the lumen, vessel or organ into the suction chamber for invaginating thereof,
the body member being adapted to accommodate a suturing instrument into or adjacent the suction chamber from a channel of the access device or from a channel extending through an elongated tubular member extending along and adjacent the access device.

2. An invaginating device as claimed in claim 1 in which the suction chamber is adapted to communicate with a channel of the access device or the channel extending through the tubular member for applying suction to the suction chamber for invaginating the tissue or the part of the wall of the lumen, vessel or organ.

3. An invaginating device as claimed in claim 1 in which the body member extends from a proximal end to a distal end, and defines a main axis extending from the proximal end to the distal end, the connector being located adjacent the proximal end of the body member, and being adapted to connect the body member to the access device with the main axis of the body member extending substantially parallel to a longitudinally extending axis of the access device adjacent the distal end thereof.

4. An invaginating device as claimed in claim 3 in which the suction chamber extends into the body member transversely of the main axis defined by the body member, and the access opening to the suction chamber faces in a direction transversely of the main axis

5. An invaginating device as claimed in claim 3 in which the suction chamber extends into the body member from the distal end thereof in an axial direction substantially parallel to the main axis defined by the body member, and the access opening to the suction chamber faces in a distal direction substantially parallel to the main axis of the body member.

6. An invaginating device as claimed in claim 1 further comprising an instrument passageway extending through the body member from the suction chamber to the connector for accommodating the suturing instrument therethrough to the suction chamber.

7. A suturing instrument comprising:
an elongated cannula extending from a proximal end to a distal end and having a suture accommodating bore extending longitudinally therethrough for accommodating a plurality of sutures therein,
the cannula terminating at its distal end in a distal piercing tip,
the sutures comprising:
a suturing thread terminating at its opposite ends in respective anchor elements, the suture thread being configured to form a loop extending from one anchor element to the other one of the anchor elements, and
a cinch clip located on the loop of the suture thread and being urgeable along the loop towards the anchor elements for progressively reducing the effective length of the suture thread between the anchor elements,
the suture accommodating bore being configured to accommodate the sutures therein with at least the anchor elements of the sutures being located in the suture accommodating bore, and
a pusher element for selectively and sequentially discharging the anchor elements of the suture from the suture accommodating bore through the distal piercing tip of the cannula.

8. A suturing apparatus for suturing two portions of a lumen, vessel or organ in a cavity of the body of a human or animal subject either interiorly in the lumen, vessel or organ or exteriorly of the lumen, vessel or organ in the cavity in the body of the human or animal subject, the suturing apparatus comprising the invaginating device as claimed in claim 1 for sequentially invaginating parts of the two portions of the lumen, vessel or organ, and a suturing instrument for inserting a suture into two invaginated parts of the respective portions of the lumen, vessel or organ.

9. A suturing apparatus as claimed in claim 8 in which the suture comprises a suture thread terminating in its opposite ends in respective anchor elements, and forming a loop between the anchor elements, and having a cinch clip located on the loop urgeable along the loop towards the anchor elements for progressively reducing the effective length of the suture thread between the anchor elements.

10. A suturing apparatus as claimed in claim 9 in which the suturing instrument comprises a cannula having a suture accommodating bore extending longitudinally therethrough for accommodating the anchor elements of the suture sequentially aligned therein and terminating at a distal end in a distal piercing tip, a pusher element urgeable distally in the suture accommodating bore for sequentially discharging the anchor elements from the suture accommodating bore through the distal piercing tip of the cannula, the distal piercing tip being adapted to extend into or adjacent the suction chamber of the invaginating device, so that when the distal piercing tip is urged through one of the invaginated parts in the suction chamber, and the pusher element is urged to discharge one of the anchor elements through the distal piercing tip of the cannula, the discharged anchor element is located on one side of the invaginated part with a part of the suture thread adjacent the anchor element extending from the anchor element through the invaginated part.

11. A suturing apparatus as claimed in claim 10 in which the pusher element terminates in a distal hook or grabber for engaging the loop of the suture and for urging the suture loop through the cinch clip for in turn urging the cinch clip along the loop towards the anchor elements to progressively reduce the effective length of the suture thread between the anchor elements.

12. A suturing apparatus as claimed in claim 8 in which the invaginating device is coupled to a distal end of an access device, and the suturing instrument is passed into or adjacent the suction chamber of the invaginating device through an instrument channel of the access device or through a tubular member extending along the access device.

13. A suturing apparatus as claimed in claim 12 in which the tubular member extending along the access device comprises a tubular member of the suturing instrument.

14. A suturing apparatus as claimed in claim 8 in which the suturing instrument comprises:
an elongated cannula extending from a proximal end to a distal end and having a suture accommodating bore extending longitudinally therethrough for accommodating a plurality of sutures therein,
the cannula terminating at its distal end in a distal piercing tip,
the sutures comprising:
a suturing thread terminating at its opposite ends in respective anchor elements, the suture thread being configured to form a loop extending from one anchor element to the other one of the anchor elements, and
a cinch clip located on the loop of the suture thread and being urgeable along the loop towards the anchor elements for progressively reducing the effective length of the suture thread between the anchor elements,
the suture accommodating bore being configured to accommodate the sutures therein with at least the anchor elements of the sutures being located in the suture accommodating bore, and
a pusher element for selectively and sequentially discharging the anchor elements of the suture from the suture accommodating bore through the distal piercing tip of the cannula.

15. A method for suturing two portions of a lumen, vessel or organ in a cavity in the body of a human or animal subject either interiorly in the lumen, vessel or organ or exteriorly of the lumen, vessel or organ in the cavity, the method comprising:
entering an access device with the invaginating device as claimed in claim 1 coupled to the distal end of the access device into the interior of the lumen, vessel or organ or into the cavity through a body orifice or through an incision in the body of the human or animal subject,
bringing the invaginating device into engagement with a surface of the wall of the lumen, vessel or organ with the suction chamber adjacent a first one of the portions to be sutured,
applying suction to the suction chamber to suction a part of the first one of the portions to be sutured into the suction chamber for invaginating thereof to form a first invaginated part,
urging a distal piercing tip of a cannula of a suturing instrument distally through the first invaginated part retained in the suction chamber,
operating the suturing instrument to discharge a first one of a pair of anchor elements of a suture from the suturing instrument to the distal side of the first invaginated part,
withdrawing the cannula proximally from the first invaginated part with a suture thread of the suture extending proximally from the first anchor element through the first invaginated part, and
releasing suction from the suction chamber to release the first invaginated part therefrom.

16. A method as claimed in claim 15 in which the method further comprises:
bringing the invaginating device into engagement with the surface of the wall of the lumen, vessel or organ with the suction chamber adjacent a second one of the portions of the lumen, vessel or organ to be sutured,
reapplying suction to the suction chamber to suction a part of the second portion to be sutured into the suction chamber for invaginating thereof to form a second invaginated part,
urging the distal piercing tip of the cannula of the suturing instrument through the second invaginated part retained in the suction chamber,
operating the suturing instrument to discharge a second one of the pair of the anchor elements of the suture from the suturing instrument to the distal side of the second invaginated part,
withdrawing the cannula proximally from the second invaginated part with the suture thread of the suture extending proximally from the second anchor element through the second invaginated part, and
releasing suction from the suction chamber to release the invaginated part therefrom.

17. A method as claimed in claim 16 in which the method comprises tightening the suture to draw the two invaginated parts together.

18. A method as claimed in claim 17 in which the suture is tightened by urging the cinch clip of the suture along the loop thereof towards the anchor elements to reduce the effective length of the suture thread between the anchor elements to draw the two invaginated parts together.

19. A method as claimed in claim 15 in which the suturing instrument is entered into the suction chamber of the invaginating device through an instrument channel of the access device or through a tubular member extending along an outer side of the access device.

20. A method as claimed in claim 15 in which the suturing instrument is secured to an outer side of the access device and extends longitudinally along the outer side of the access device.

21. A method as claimed in claim 15 in which the lumen, vessel or organ to be sutured comprises the stomach of the subject.

22. A method as claimed in claim 15 in which the method is adapted for carrying out a sleeve gastroplasty procedure, and a plurality of pairs of invaginated parts are formed on the front and rear walls of the stomach, with one of each pair of invaginated parts being formed on the front wall of the stomach, and the other one of each pair of the invaginated parts being formed on the rear wall of the stomach, each pair of the invaginated parts being sutured together.

23. A method for suturing two portions of a lumen, vessel or organ in a cavity in the body of a human or animal subject, either internally in the interior of the lumen, vessel or organ, or externally of the lumen, vessel or organ in the interior of the cavity, the method comprising:
invaginating at least one part of each of the portions to be sutured of the lumen, vessel or organ to form a pair of the invaginated parts, and
suturing the invaginated parts of each pair thereof together.

24. A method as claimed in claim 23 in which each invaginated part is retained invaginated by applying a band around the invaginated part adjacent an end thereof at which the invaginated part extends from the corresponding portion of the lumen, vessel or organ to be sutured.
